(19) United States
(12) Patent Application Publication
(10) Pub. No.: US 2021/0355166 A1
(43) Pub. Date: Nov. 18, 2021

(54) ANALGESICS AND METHODS OF USE THEREOF

(71) Applicants: The University of Sydney, Sydney (AU); The University of Queensland, St. Lucia (AU)

(72) Inventors: MacDonald Christie, Sydney (AU); Paul Alewood, Brisbane (AU); Robert Capon, Brisbane (AU); Zoltan Dekan, Brisbane (AU)

(21) Appl. No.: 15/733,884

(22) PCT Filed: May 30, 2019
(86) PCT No.: PCT/AU2019/050550

§ 371 Date: Nov. 30, 2020

(30) Foreign Application Priority Data
May 31, 2018 (AU) 2018901944

(51) Int. Cl. C07K 9/00; A61P 25/04

(52) U.S. Cl. CPC C07K 9/001; A61P 25/04

(57) ABSTRACT

The present invention relates to peptides with alternating stereochemistry. In particular, the invention relates to peptides comprising alternating stereochemistry of (LDLD) in the first four amino acid residues. The invention further contemplates the use of peptides with alternating stereochemistry in treating pain.

The application claims the benefit of Australian Provisional Application No. 2018901944, filed on 31 May 2018, entitled “Analgesics and Methods of Use Thereof”. The entire contents of the foregoing are hereby incorporated by reference herein. This specification includes 28 figures, some of which include multiple parts.

### FIELD OF THE INVENTION

The present invention relates to peptides with alternating stereochemistry. In particular, the invention relates to peptides comprising alternating stereochemistry of (LDLD) in the first four amino acid residues. The invention further contemplates the use of peptides with alternating stereochemistry for use in treating pain.

### BACKGROUND OF THE INVENTION

Any discussion of the prior art throughout the specification should in no way be considered as an admission that such prior art is widely known or forms part of common general knowledge in the field.

Short bioactive peptides (4-50 residues) display great promise for both their selectivity and novel pharmacological properties. Since the discovery of the relatively non-selective opioid peptides, enkephalins and endorphins in the 1970s, other putative endogenous ligands have been discovered including the tetrapeptide endomorphins that target the μ-opioid receptor (MOPr) with selectivity over the related κ-opioid (KOPr) and δ-opioid (DOPr) subtypes. Nature also plays a discovery role delivering dermorphin and deltorphin II, heptapeptides isolated from frog skin, that are potent, selective agonists for MOPr and DOPr, respectively; interestingly, both contain a D-alanine in the second position (Kreil, G., In Antimicrobial peptides. Ciba Foundation Symposium, no. 186, pp. 77-90. 1994). Synthetic modifications to mammalian opioid peptides using a similar strategy has also delivered enhanced biological stability and receptor selectivity. For example, introduction of D-alanine stabilises the enkephalins to proteolysis and further substitutions near their C-terminal yields highly stable, selective MOPr agonists such as DAMGO ([D-Ala², N-MePhe⁴, Gly⁵-ol]-enkephalin). Although opioid peptide agonists and analogues with high affinity and selectivity for opioid receptor types have been isolated from nature or developed, all known potent endogenous and synthetic opioid peptides studied so far are more or less unbiased or arrestin-biased, and all produce robust MOPr internalisation (e.g. Thompson G L et al (2015) Molecular Pharmacology 88:335-346). One example of a cyclic peptide has recently been reported to be G-biased (Piekielna-Ciesielska J et al (2018) Peptides 101:227-233.

Agonists at the MOPr are extremely important drugs for the management of pain but their use often leads to undesirable effects, including respiratory depression, constipation, and tolerance. There is also the potential for abuse of MOPr agonists. Biased agonism describes the ability of an agonist of a G protein-coupled receptor (GPCR) to differently agonize the GPCR to couple to distinct downstream signaling pathways. Biased opioids that differentially signal via G-proteins versus β-arrestin recruitment are of increasing interest because absence of β-arrestin recruitment may improve the side effect profile. For example, the analgesic effects of morphine were enhanced and prolonged in β-arrestin-2 knockout mice, whereas morphine-induced respiratory depression and acute constipation were diminished (Raehal, K. et al., Journal of Pharmacology and Experimental Therapeutics 314, no. 3 (2005): 1195-1201). Similarly, a G protein-biased MOPr agonist, oliceridine (TRV130), which is a potent analgesic in mice and rats produces less respiratory depression and gastrointestinal dysfunction than morphine (DeWire, S. et al., Journal of Pharmacology and Experimental Therapeutics 344, no. 3 (2013): 708-717). Clinical trials of oliceridine have shown reduced respiratory impairment in comparison to morphine for equi-analgesic doses (Singla, N. et al., Journal of Pain Research 10 (2017): 2413). Reduced respiratory depression would provide improved safety in overdose situations, potentially reducing the morbidity and mortality burden of opioid overdoses that are now at epidemic proportions in many jurisdictions. Therefore, there is a need to develop new analgesic drugs with fewer of the unwanted effects associated with classic opioids such as morphine.

It is an object of the present invention to overcome or ameliorate at least one of the disadvantages of the prior art, or to provide a useful alternative.

### SUMMARY OF THE INVENTION

While opioid families have been identified in vertebrates and a number of other animals, opioid-related peptides have not been identified in lower eukaryotes. The invention relates to tetrapeptides, FvVf-OH (designated Bilaid A), FvVy-OH (designated Bilaid B), and YvVf-OH (designated Bilaid C), isolated from an Australian estuarine isolate of Penicillium sp. MST-MF667, which was initially reported as an Australian marine-derived Penicillium bilaii, and derivatives of these peptides. The peptides share alternating stereochemistry (LDLD) of the four amino acid residues.

In a first aspect, the invention provides an isolated peptide comprising Formula I

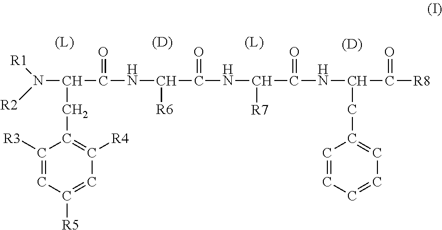

wherein, counting from the N-terminus, the first and third amino acid residues are L-amino acid residues and the second and fourth amino acid residues are D-amino acid residues; wherein
R¹ is hydrogen, C₁-C₃ alkyl, or a bio-reversible moiety optionally comprising a sugar moiety;
R² is hydrogen, C₁-C₃ alkyl, or a bio-reversible moiety optionally comprising a sugar moiety;
wherein R¹ and R² may together form one bio-reversible moiety optionally comprising a sugar moiety;
   R³ and R⁴ are independently selected from hydrogen or C₁-C₃ alkyl, preferably —CH₃;
   R⁵ is hydrogen, —OH, or a bio-reversible moiety optionally comprising a sugar moiety;
   R⁶ is a side chain of an amino acid or C₁-C₆ alkyl, preferably C₁-C₄ alkyl, more preferably —CH(CH₃)₂;
   R⁷ is a side chain of an amino acid or C₁-C₆ alkyl, preferably C₁-C₄ alkyl, more preferably —CH(CH₃)₂;
   R⁸ is —OH, —NH₂, —O(C₁-C₃ alkyl),

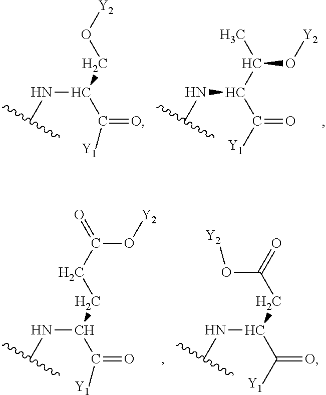

or 1 to about 30 L-amino acid residues;
Y₁ is —OH, —NH₂, or 1 to about 30 L-amino acid residues;
Y₂ is hydrogen or a sugar moiety, preferably a disaccharide moiety; and
wherein when R^{a} is 1 to about 30 L-amino acid residues (1) the L-amino acid residues are optionally residues that may be optionally glycosylated with a sugar moiety, preferably a disaccharide moiety, and (2) the C-terminus is optionally amidated.

In an embodiment of the first aspect of the invention,

R¹ is hydrogen or a bio-reversible moiety optionally comprising a sugar moiety;
R² is hydrogen or a bio-reversible moiety optionally comprising a sugar moiety;
wherein R¹ and R² may together form one bio-reversible moiety optionally comprising a sugar moiety;
R³ and R⁴ are independently selected from hydrogen or C₁-C₃ alkyl, preferably —CH₃;
R⁵ is hydrogen, —OH, or a bio-reversible moiety optionally comprising a sugar moiety;
R⁶ is a side chain of an amino acid or C₁-C₆ alkyl, preferably C₁-C₄ alkyl, more preferably —CH(CH₃)₂;
R⁷ is a side chain of an amino acid or C₁-C₆ alkyl, preferably C₁-C₄ alkyl, more preferably —CH(CH₃)₂;
R⁸ is —OH, —NH₂, —O(C₁-C₃ alkyl),

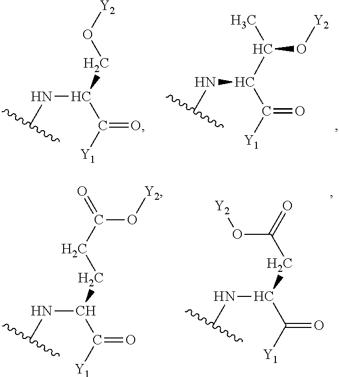

or 1 to about 30 L-amino acid residues;
Y₁ is —OH, —NH₂, or 1 to about 30 L-amino acid residues;
Y₂ is hydrogen or a sugar moiety, preferably a disaccharide moiety; and
wherein when R⁸ is 1 to about 30 L-amino acid residues (1) the L-amino acid residues are optionally residues that may be optionally glycosylated with a sugar moiety, preferably a disaccharide moiety, and (2) the C-terminus is optionally amidated.

L-amino acid residues that may be optionally glycosylated include residues that may be N-glycosylated (also referred to as N-linked glycosylation) such as L-asparagine, L-glutamine, L-lysine, L-histidine, and L-arginine; O-glycosylated (also referred to as O-linked glycosylation) such as L-serine, L-threonine, L-tyrosine, L-hydroxylysine, and L-hydroxyproline; S-glycosylated (also referred to as S-linked glycosylation) such as L-cysteine; C-glycosylation (also referred to as C-linked glycosylation) such as L-tryptophan; and Se-glycosylated (also referred to as Se-linked glycosylation) such as L-selenocysteine.

Peptides according to the first aspect of the invention include peptides wherein R⁸ is 1 to about 25 L-amino acid residues, 1 to about 20 L-amino acid residues, 1 to about 15 L-amino acid residues, 1 to about 11 L-amino acid residues, or 1 to about 5 L-amino acid residues.

Peptides according to the first aspect of the invention include peptides wherein Y₁ is 1 to about 25 L-amino acid residues, 1 to about 20 L-amino acid residues, 1 to about 15 L-amino acid residues, 1 to about 11 L-amino acid residues, or 1 to about 5 L-amino acid residues.

Peptides according to the first aspect of the invention include peptides wherein R⁸ is 1 L-amino acid residue wherein the L-amino acid residue is preferably a residue that may be optionally glycosylated.

In peptides according to the first aspect of the invention comprising a bio-reversible moiety, the bio-reversible moiety may be any known in the art, including, but not limited to, a carbonate, carbamate, imine, ether, ester, and amide moiety. In peptides according to the first aspect of the invention comprising a bio-reversible moiety, the bio-reversible moiety optionally comprises a sugar moiety, preferably a disaccharide.

In certain embodiments, in peptides according to the first aspect of the invention, one of R¹ or R² is a bio-reversible moiety that is glycosylated with a sugar moiety, preferably a disaccharide moiety.

In certain embodiments, in peptides according to the first aspect of the invention, R⁶ is a side chain of an amino acid and/or R⁷ is a side chain of an amino acid. In certain embodiments, R⁶ is a side chain of a threonine and/or R⁷ is a side chain of a threonine. In certain embodiments, one of R⁶ or R⁷ is a side chain of a threonine and one R⁶ or R⁷ is a side chain of a valine. In certain embodiments, both R⁶ and R⁷ are a side chain of a threonine. In certain embodiments, R⁶ is a side chain of a valine and/or R⁷ is a side chain of a valine. In certain embodiments, both R⁶ and R⁷ are a side chain of a valine.

Preferably, in peptides according to the first aspect of the invention R¹ and R² are hydrogen.

In a second aspect, the invention provides an isolated peptide comprising Formula I, wherein, counting from the N-terminus, the first and third amino acid residues are L-amino acid residues and the second and fourth amino acid residues are D-amino acid residues; wherein

R¹ is hydrogen, C₁-C₃ alkyl, or a bio-reversible moiety optionally comprising a sugar moiety;
R² is hydrogen, C₁-C₃ alkyl, or a bio-reversible moiety optionally comprising a sugar moiety;
wherein R¹ and R² may together form one bio-reversible moiety optionally comprising a sugar moiety;
   R³ and R⁴ are independently selected from hydrogen or C₁-C₃ alkyl, preferably —CH₃;
   R⁵ is hydrogen, —OH, or a bio-reversible moiety optionally comprising a sugar moiety;
   R⁶ is C₁-C₆ alkyl, preferably C₁-C₄ alkyl, more preferably —CH(CH₃)₂;
   R⁷ is C₁-C₆ alkyl, preferably C₁-C₄ alkyl, more preferably —CH(CH₃)₂;
   R⁸ is —OH, —NH₂, —O(C₁-C₃ alkyl)

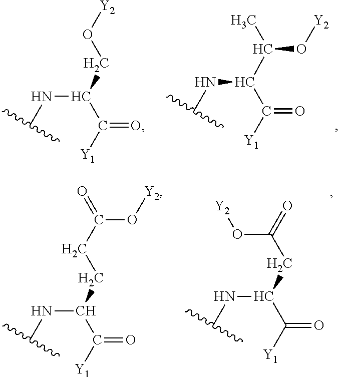

or 1 to about 30 L-amino acid residues;
Y₁ is —NH₂, or 1 to about 30 L-amino acid residues;
Y₂ is hydrogen or a sugar moiety, preferably a disaccharide moiety; and
wherein when R⁸ is 1 to about 30 L-amino acid residues (1) the L-amino acid residues are optionally residues that may be optionally glycosylated with a sugar moiety, preferably a disaccharide moiety, and (2) the C-terminus is optionally amidated.

In an embodiment of the second aspect of the invention,

R¹ is hydrogen or a bio-reversible moiety optionally comprising a sugar moiety;
R² is hydrogen or a bio-reversible moiety optionally comprising a sugar moiety;
wherein R¹ and R² may together form one bio-reversible moiety optionally comprising a sugar moiety;
R³ and R⁴ are independently selected from hydrogen or C₁-C₃ alkyl, preferably —CH₃;
R⁵ is hydrogen, —OH, or a bio-reversible moiety optionally comprising a sugar moiety;
R⁶ is C₁-C₆ alkyl, preferably C₁-C₄ alkyl, more preferably —CH(CH₃)₂;
R⁷ is C₁-C₆ alkyl, preferably C₁-C₄ alkyl, more preferably —CH(CH₃)₂;
R⁸ is —OH, —NH₂, —O(C₁-C₃ alkyl)

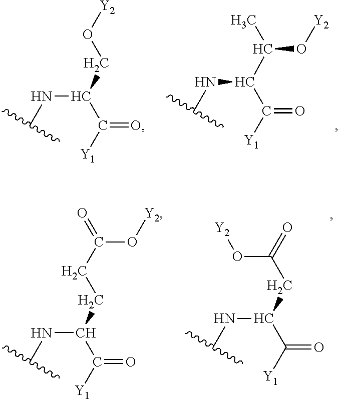

or 1 to about 30 L-amino acid residues;
Y₁ is —OH, —NH₂, or 1 to about 30 L-amino acid residues;
Y₂ is hydrogen or a sugar moiety, preferably a disaccharide moiety; and
wherein when R⁸ is 1 to about 30 L-amino acid residues (1) the L-amino acid residues are optionally residues that may be optionally glycosylated with a sugar moiety, preferably a disaccharide moiety, and (2) the C-terminus is optionally amidated.

L-amino acid residues that may be optionally glycosylated include residues that may be N-glycosylated (also referred to as N-linked glycosylation) such as L-asparagine, L-glutamine, L-lysine, L-histidine, and L-arginine; O-glycosylated (also referred to as O-linked glycosylation) such as L-serine, L-threonine, L-tyrosine, L-hydroxylysine, and L-hydroxyproline; S-glycosylated (also referred to as S-linked glycosylation) such as L-cysteine; C-glycosylation (also referred to as C-linked glycosylation) such as L-tryptophan; and Se-glycosylated (also referred to as Se-linked glycosylation) such as L-selenocysteine.

Peptides according to the second aspect of the invention include peptides wherein R⁸ is 1 to about 25 L-amino acid residues, 1 to about 20 L-amino acid residues, 1 to about 15 L-amino acid residues, 1 to about 11 L-amino acid residues, or 1 to about 5 L-amino acid residues.

Peptides according to the second aspect of the invention include peptides wherein Y₁ is 1 to about 25 L-amino acid residues, 1 to about 20 L-amino acid residues, 1 to about 15 L-amino acid residues, 1 to about 11 L-amino acid residues, or 1 to about 5 L-amino acid residues.

Peptides according to the second aspect of the invention include peptides wherein R⁸ is 1 L-amino acid residue wherein the L-amino acid residue is preferably a residue that may be optionally glycosylated.

In peptides according to the second aspect of the invention comprising a bio-reversible moiety, the bio-reversible moiety may be any known in the art, including, but not limited to, a carbonate, carbamate, imine, ether, ester, and amide moiety. In peptides according to the second aspect of the invention comprising a bio-reversible moiety, the bio-reversible moiety optionally comprises a sugar moiety, preferably a disaccharide.

In certain embodiments, in peptides according to the second aspect of the invention, one of R¹ or R² is a bio-reversible moiety that is glycosylated with a sugar moiety, preferably a disaccharide moiety.

Preferably, in peptides according to the second aspect of the invention R¹ and R² are hydrogen.

In a third aspect, the invention provides an isolated peptide comprising Formula I, wherein, counting from the N-terminus, the first and third amino acid residues are L-amino acid residues and the second and fourth amino acid residues are D-amino acid residues; wherein

R¹ is hydrogen, C₁-C₃ alkyl, or a bio-reversible moiety optionally comprising a sugar moiety;
R² is hydrogen, C₁-C₃ alkyl, or a bio-reversible moiety optionally comprising a sugar moiety;
wherein R¹ and R² may together form one bio-reversible moiety optionally comprising a sugar moiety;
   R³ and R⁴ are independently selected from hydrogen or —CH₃;
   R⁵ is hydrogen, —OH, or a bio-reversible moiety optionally comprising a sugar moiety;
   R⁶ is a side chain of an amino acid or C₁-C₆ alkyl, preferably C₁-C₄ alkyl, more preferably —CH(CH₃)₂;
   R⁷ is a side chain of an amino acid or C₁-C₆ alkyl, preferably C₁-C₄ alkyl, more preferably —CH(CH₃)₂; and
   R⁸ is —OH, —NH₂, —O(C₁-C₃ alkyl),

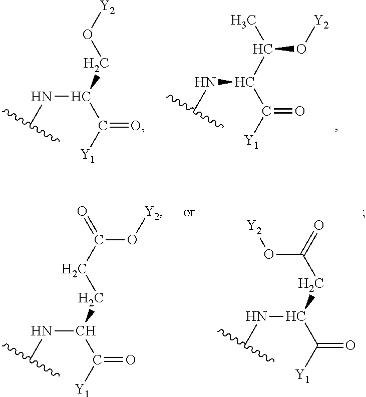

Y₁ is —OH, or —NH₂; and
Y₂ is hydrogen or a sugar moiety, preferably a disaccharide moiety.

In an embodiment of the third aspect of the invention,

R¹ is hydrogen or a bio-reversible moiety optionally comprising a sugar moiety;
R² is hydrogen or a bio-reversible moiety optionally comprising a sugar moiety;
wherein R¹ and R² may together form one bio-reversible moiety optionally comprising a sugar moiety;
R³ and R⁴ are independently selected from hydrogen or —CH₃;
R⁵ is hydrogen, —OH, or a bio-reversible moiety optionally comprising a sugar moiety;
R⁶ is a side chain of an amino acid or C₁-C₆ alkyl, preferably C₁-C₄ alkyl, more preferably —CH(CH₃)₂;
R⁷ is a side chain of an amino acid or C₁-C₆ alkyl, preferably C₁-C₄ alkyl, more preferably —CH(CH₃)₂; and
R⁸ is —OH, —NH₂, —O(C₁-C₃ alkyl),

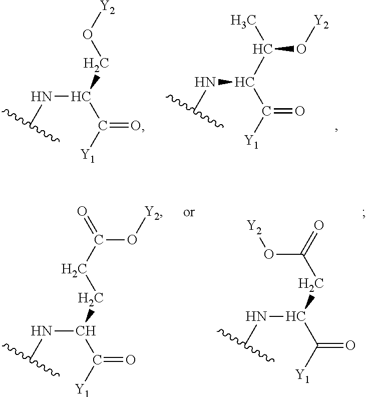

Y₁ is —OH, or —NH₂; and
Y₂ is hydrogen or a sugar moiety, preferably a disaccharide moiety.

Peptides according to the third aspect of the invention optionally comprise additional L-amino acid residues on the C-terminus of the peptide, wherein said additional L-amino acid residues are optionally glycosylated. Preferably, the peptides of the third aspect of the invention optionally comprise about 5, about 8, about 11, about 12, about 20 or about 26 additional L-amino acid residues on the C-terminus. The peptide optionally comprises additional L-amino acid residues that may be optionally glycosylated, including residues that may be N-glycosylated (also referred to as N-linked glycosylation) such as L-asparagine, L-glutamine, L-lysine, L-histidine, and L-arginine; O-glycosylated (also referred to as O-linked glycosylation) such as L-serine, L-threonine, L-tyrosine, L-hydroxylysine, and L-hydroxyproline; S-glycosylated (also referred to as S-linked glycosylation) such as L-cysteine; C-glycosylation (also referred to as C-linked glycosylation) such as L-tryptophan; and Se-glycosylated (also referred to as Se-linked glycosylation) such as L-selenocysteine. The C-terminus is optionally amidated.

In peptides according to the third aspect of the invention comprising a bio-reversible moiety, the bio-reversible moiety may be any known in the art, including, but not limited to, a carbonate, carbamate, imine, ether, ester, and amide moiety. In peptides according to the third aspect of the invention comprising a bio-reversible moiety, the bio-reversible moiety optionally comprises a sugar moiety, preferably a disaccharide.

In certain embodiments, in peptides according to the third aspect of the invention, one of R¹ or R² is a bio-reversible moiety that is glycosylated with a sugar moiety, preferably a disaccharide moiety.

In certain embodiments, in peptides according to the third aspect of the invention, R⁶ is a side chain of an amino acid and/or R⁷ is a side chain of an amino acid. In certain embodiments, R⁶ is a side chain of a threonine and/or R⁷ is a side chain of a threonine. In certain embodiments, one of R⁶ or R⁷ is a side chain of a threonine and one R⁶ or R⁷ is a side chain of a valine. In certain embodiments, both R⁶ and R⁷ are a side chain of a threonine. In certain embodiments, R⁶ is a side chain of a valine and/or R⁷ is a side chain of a valine. In certain embodiments, both R⁶ and R⁷ are a side chain of a valine.

Preferably, in peptides according to the third aspect of the invention R¹ and R² are hydrogen.

In a fourth aspect, the invention provides a peptide comprising Formula I, wherein, counting from the N-terminus, the first and third amino acid residues are L-amino acid residues and the second and fourth amino acid residues are D-amino acid residues; wherein

R¹ is hydrogen or a bio-reversible moiety optionally comprising a sugar moiety;
R² is hydrogen or a bio-reversible moiety optionally comprising a sugar moiety;
wherein R¹ and R² may together form one bio-reversible moiety optionally comprising a sugar moiety;
   R³ and R⁴ are independently selected from hydrogen or —CH₃;
   R⁵ is hydrogen, —OH, or a bio-reversible moiety optionally comprising a sugar moiety;
   R⁶ is C₁-C₄ alkyl, preferably —CH(CH₃)₂;
   R⁷ is C₁-C₄ alkyl; preferably —CH(CH₃)₂; and
   R⁸ is —NH₂, —O(C₁-C₃ alkyl),

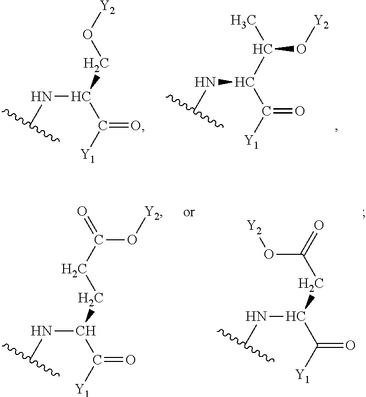

Y₁ is —OH or —NH₂; and
Y₂ is hydrogen or a sugar moiety, preferably a disaccharide moiety.

Peptides according to the fourth aspect of the invention optionally comprise additional L-amino acid residues on the C-terminus of the peptide, wherein said additional L-amino acid residues are optionally glycosylated. Preferably, the peptides of the fourth aspect of the invention optionally comprise about 5, about 8, about 11, about 12, about 20 or about 26 additional L-amino acid residues on the C-terminus. The peptide optionally comprises additional L-amino acid residues that may be optionally glycosylated include residues that may be N-glycosylated (also referred to as N-linked glycosylation) such as L-asparagine, L-glutamine, L-lysine, L-histidine, and L-arginine; O-glycosylated (also referred to as O-linked glycosylation) such as L-serine, L-threonine, L-tyrosine, L-hydroxylysine, and L-hydroxyproline; S-glycosylated (also referred to as S-linked glycosylation) such as L-cysteine; C-glycosylation (also referred to as C-linked glycosylation) such as L-tryptophan; and Se-glycosylated (also referred to as Se-linked glycosylation) such as L-selenocysteine. The C-terminus is optionally amidated.

In peptides according to the fourth aspect of the invention comprising a bio-reversible moiety, the bio-reversible moiety may be any known in the art, including, but not limited to, a carbonate, carbamate, imine, ether, ester, and amide moiety. In peptides according to the fourth aspect of the invention comprising a bio-reversible moiety, the bio-reversible moiety optionally comprises a sugar moiety, preferably a disaccharide.

In certain embodiments, in peptides according to the fourth aspect of the invention, one of R¹ or R² is a bio-reversible moiety that is glycosylated with a sugar moiety, preferably a disaccharide moiety.

Preferably, in peptides according to the fourth aspect of the invention R¹ and R² are hydrogen.

In a fifth aspect, the invention provides a peptide comprising Formula I, wherein, counting from the N-terminus, the first and third amino acid residues are L-amino acid residues and the second and fourth amino acid residues are D-amino acid residues; wherein

R¹ is hydrogen or a bio-reversible moiety optionally comprising a sugar moiety;
R² is hydrogen or a bio-reversible moiety optionally comprising a sugar moiety;
wherein when one of R¹ or R² is hydrogen and one of R¹ or R² is a bio-reversible moiety, the bio-reversible moiety is preferably —C(═O)OZ₃ or —C(═O)OCH₂OC(═O)Z₂;
   Z₁ is C₁-C₆ alkyl or aryl, preferably Z₂ is —CH₂CH₃;
   Z₂ is C₁-C₆ alkyl or aryl, preferably Z₂ is —CH₃;
wherein R¹ and R² may together form one bio-reversible moiety, wherein preferably the bio-reversible moiety is

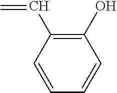

(imine moiety), or ═N═N (azido moiety);
   R³ and R⁴ are —CH₃;

### R⁵ is OH;

R⁶ is a C₁-C₄ alkyl;
R⁷ is a C₁-C₄ alkyl; and
R⁸ is —OH, —NH₂,

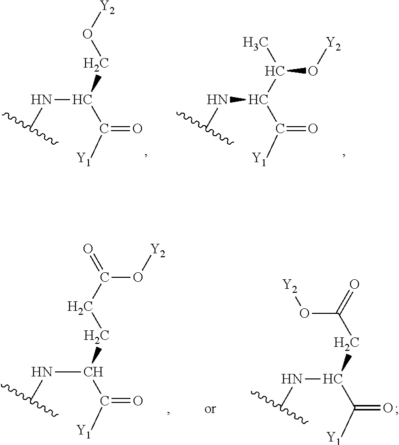

Y₁ is —OH, or —NH₂; and
Y₂ is hydrogen or a sugar moiety, preferably a disaccharide moiety.

Peptides according to the fifth aspect of the invention optionally comprise additional L-amino acid residues on the C-terminus of the peptide, wherein said additional L-amino acid residues are optionally glycosylated. Preferably, the peptides of the fifth aspect of the invention optionally comprise about 5, about 8, about 11, about 12, about 20 or about 26 additional L-amino acid residues on the C-terminus. The peptide optionally comprises additional L-amino acid residues that may optionally be glycosylated, including residues that may be N-glycosylated (also referred to as N-linked glycosylation) such as L-asparagine, L-glutamine, L-lysine, L-histidine, and L-arginine; O-glycosylated (also referred to as O-linked glycosylation) such as L-serine, L-threonine, L-tyrosine, L-hydroxylysine, and L-hydroxyproline; S-glycosylated (also referred to as S-linked glycosylation) such as L-cysteine; C-glycosylation (also referred to as C-linked glycosylation) such as L-tryptophan; and Se-glycosylated (also referred to as Se-linked glycosylation) such as L-selenocysteine. The C-terminus is optionally amidated.

In peptides according to the fifth aspect of the invention comprising a bio-reversible moiety, the bio-reversible moiety may be any known in the art, including, but not limited to, a carbonate, carbamate, imine, ether, ester, and amide moiety. In peptides according to the fifth aspect of the invention comprising a bio-reversible moiety, the bio-reversible moiety optionally comprises a sugar moiety, preferably a disaccharide.

In certain embodiments, in peptides according to the fifth aspect of the invention, one of R¹ or R² is a bio-reversible moiety that is glycosylated with a sugar moiety, preferably a disaccharide moiety.

Preferably, in peptides according to the fifth aspect of the invention R¹ and R² are hydrogen.

In a sixth aspect, the invention provides a peptide comprising Formula I, wherein, counting from the N-terminus, the first and third amino acid residues are L-amino acid residues and the second and fourth amino acid residues are D-amino acid residues; wherein

R¹ is hydrogen or a bio-reversible moiety optionally comprising a sugar moiety;
R² is hydrogen or a bio-reversible moiety optionally comprising a sugar moiety;
wherein when one of R¹ or R² is hydrogen and one of R¹ or R² is a bio-reversible moiety, the bio-reversible moiety is preferably —C(═O)OZ₃ or —C(═O)OCH₂OC(═O)Z₂;
   Z₁ is C₁-C₆ alkyl or aryl, preferably Z₂ is —CH₂CH₃;
   Z₂ is C₁-C₆ alkyl or aryl, preferably Z₂ is —CH₃;
wherein R¹ and R² may together form one bio-reversible moiety, wherein preferably the bio-reversible moiety is

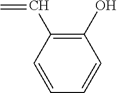

(imine moiety), or ═N═N (azido moiety);
   R³ and R⁴ are —CH₃;

### R⁵ is OH;

R⁶ is —CH(CH₃)₂;
R⁷ is —CH(CH₃)₂; and
R⁸ is —OH, —NH₂,

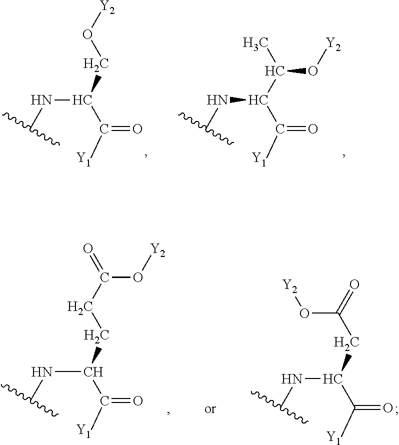

Y₁ is —OH, or —NH₂; and
Y₂ is hydrogen or a sugar moiety, preferably a disaccharide moiety.

Peptides according to the sixth aspect of the invention optionally comprise additional L-amino acid residues on the C-terminus of the peptide, wherein said additional L-amino acid residues are optionally glycosylated. Preferably, the peptides of the sixth aspect of the invention optionally comprise about 5, about 8, about 11, about 12, about 20 or about 26 additional L-amino acid residues on the C-terminus. The peptide optionally comprises additional L-amino acid residues that may optionally be glycosylated, including residues that may be N-glycosylated (also referred to as N-linked glycosylation) such as L-asparagine, L-glutamine, L-lysine, L-histidine, and L-arginine; O-glycosylated (also referred to as O-linked glycosylation) such as L-serine, L-threonine, L-tyrosine, L-hydroxylysine, and L-hydroxyproline; S-glycosylated (also referred to as S-linked glycosylation) such as L-cysteine; C-glycosylation (also referred to as C-linked glycosylation) such as L-tryptophan; and Se-glycosylated (also referred to as Se-linked glycosylation) such as L-selenocysteine. The C-terminus is optionally amidated.

In peptides according to the sixth aspect of the invention comprising a bio-reversible moiety, the bio-reversible moiety may be any known in the art, including, but not limited to, a carbonate, carbamate, imine, ether, ester, and amide moiety. In peptides according to the sixth aspect of the invention comprising a bio-reversible moiety, the bio-reversible moiety optionally comprises a sugar moiety, preferably a disaccharide.

In certain embodiments, in peptides according to the sixth aspect of the invention, one of R¹ or R² is a bio-reversible moiety that is glycosylated with a sugar moiety, preferably a disaccharide moiety.

Preferably, in peptides according to the sixth aspect of the invention R¹ and R² are hydrogen.

In a seventh aspect, the invention provides a peptide comprising Formula I, wherein, counting from the N-terminus, the first and third amino acid residues are L-amino acid residues and the second and fourth amino acid residues are D-amino acid residues; wherein

R¹ is hydrogen;
R² is hydrogen;
R³ and R⁴ are —CH₃;

### R⁵ is OH;

R⁶ is —CH(CH₃)₂;
R⁷ is —CH(CH₃)₂; and
R⁸ is —OH, —NH₂,

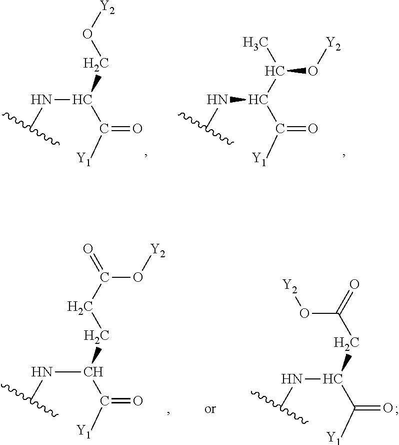

Y₁ is —OH, or —NH₂; and
Y₂ is hydrogen or a sugar moiety, preferably a disaccharide moiety.

Peptides according to the seventh aspect of the invention optionally comprise additional L-amino acid residues on the C-terminus of the peptide, wherein said additional L-amino acid residues are optionally glycosylated. Preferably, the peptides of the seventh aspect of the invention optionally comprise about 5, about 8, about 11, about 12, about 20 or about 26 additional L-amino acid residues on the C-terminus. The peptide optionally comprises additional L-amino acid residues that may optionally be glycosylated, including residues that may be N-glycosylated (also referred to as N-linked glycosylation) such as L-asparagine, L-glutamine, L-lysine, L-histidine, and L-arginine; O-glycosylated (also referred to as O-linked glycosylation) such as L-serine, L-threonine, L-tyrosine, L-hydroxylysine, and L-hydroxyproline; S-glycosylated (also referred to as S-linked glycosylation) such as L-cysteine; C-glycosylation (also referred to as C-linked glycosylation) such as L-tryptophan; and Se-glycosylated (also referred to as Se-linked glycosylation) such as L-selenocysteine. The C-terminus is optionally amidated.

In an eighth aspect, the invention provides an isolated peptide comprising Formula I, wherein, counting from the N-terminus, the first and third amino acid residues are L-amino acid residues and the second and fourth amino acid residues are D-amino acid residues; wherein

R¹ is hydrogen, a single bond, or a C₁-C₃ alkyl, preferably hydrogen or —CH₃;
R² is hydrogen, a single bond, or a C₁-C₃ alkyl, preferably hydrogen or —CH₃;
R³ and R⁴ are independently selected from hydrogen or C₁-C₃ alkyl, preferably —CH₃;
R⁵ is hydrogen, —OH, or —O(C₁-C₄)alkyl;
R⁶ is a side chain of an amino acid, preferably a side chain of a valine or threonine residue, or C₁-C₆ alkyl, preferably C₁-C₄ alkyl, more preferably —CH(CH₃)₂;
R⁷ is a side chain of an amino acid, preferably a side chain of a valine or threonine residue, or C₁-C₆ alkyl, preferably C₁-C₄ alkyl, more preferably —CH(CH₃)₂;
R⁸ is —OH, —NH₂, —O(C₁-C₃ alkyl),

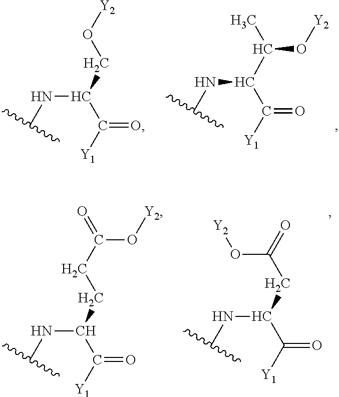

1 to about 30 L-amino acid residues, or a linker;
Y₁ is —OH, —NH₂, or 1 to about 30 L-amino acid residues;
Y₂ is hydrogen or a sugar moiety, preferably a disaccharide moiety;
   wherein when R⁸ is 1 to about 30 L-amino acid residues (1) the L-amino acid residues are optionally residues that may be optionally glycosylated with a sugar moiety, preferably a disaccharide moiety, and (2) the C-terminus is optionally amidated;
   wherein when R⁸ is a linker, the linker comprises a sugar moiety, preferably a disaccharide moiety such as lactose, and
   wherein when one of R¹ or R² is a single bond, one of R¹ and R² is hydrogen and the single bond is a peptide bond to an L-amino acid residue that may optionally be N-terminally alkylated, preferably singly methylated.

L-amino acid residues that may be optionally glycosylated include residues that may be N-glycosylated (also referred to as N-linked glycosylation) such as L-asparagine, L-glutamine, L-lysine, L-histidine, and L-arginine; O-glycosylated (also referred to as O-linked glycosylation) such as L-serine, L-threonine, L-tyrosine, L-hydroxylysine, and L-hydroxyproline; S-glycosylated (also referred to as S-linked glycosylation) such as L-cysteine; C-glycosylation (also referred to as C-linked glycosylation) such as L-tryptophan; and Se-glycosylated (also referred to as Se-linked glycosylation) such as L-selenocysteine.

Peptides according to the eighth aspect of the invention include peptides wherein R⁸ is 1 to about 25 L-amino acid residues, 1 to about 20 L-amino acid residues, 1 to about 15 L-amino acid residues, 1 to about 11 L-amino acid residues, or 1 to about 5 L-amino acid residues.

Peptides according to the eighth aspect of the invention include peptides wherein Y₁ is 1 to about 25 L-amino acid residues, 1 to about 20 L-amino acid residues, 1 to about 15 L-amino acid residues, 1 to about 11 L-amino acid residues, or 1 to about 5 L-amino acid residues.

Peptides according to the eighth aspect of the invention include peptides wherein R⁸ is 1 L-amino acid residue wherein the L-amino acid residue is preferably a residue that may be optionally glycosylated.

In peptides according to the eighth aspect of the invention comprising a bio-reversible moiety, the bio-reversible moiety may be any known in the art, including, but not limited to, a carbonate, carbamate, imine, ether, ester, and amide moiety. In peptides according to the eighth aspect of the invention comprising a bio-reversible moiety, the bio-reversible moiety optionally comprises a sugar moiety, preferably a disaccharide.

In certain embodiments, in peptides according to the eighth aspect of the invention, one of R¹ or R² is a bio-reversible moiety that is glycosylated with a sugar moiety, preferably a disaccharide moiety.

In certain embodiments, in peptides according to the eighth aspect of the invention, R⁶ is a side chain of an amino acid and/or R⁷ is a side chain of an amino acid. In certain embodiments, R⁶ is a side chain of a threonine and/or R⁷ is a side chain of a threonine. In certain embodiments, one of R⁶ or R⁷ is a side chain of a threonine and one R⁶ or R⁷ is a side chain of a valine. In certain embodiments, both R⁶ and R⁷ are a side chain of a threonine. In certain embodiments, R⁶ is a side chain of a valine and/or R⁷ is a side chain of a valine. In certain embodiments, both R⁶ and R⁷ are a side chain of a valine.

In peptides according to the eighth aspect of the invention comprising a linker as R⁸, the linker is not particularly limited. Suitable linkers include amino acid based linkers, including but not limited to single amino acid linkers, such as L-Cysteine, L-lysine, L-Serine, L-threonine, and the like, peptide based linkers including but not limited to L-Valine-L-Citrulline, L-Phe-L-Lys, L-Glutamic acid-L-Valine-L-Citrulline, and the like, amino acid comprising linkers, including but not limited to valine-citrulline-p-aminocarbamate (VC-PABC), and the like, and maleimide based linkers, including but not limited to maleimidocaproyl, maleimidomethyl cyclohexane-1-carboxylate and the like; as well as combinations of such linkers such as maleimidocaproyl-valine-citrulline-p-aminocarbamate, as well as amino and carboxy group containing linkers such as 6-aminohexanoic acid, and the like. The skilled person will appreciate that maleimide based linkers may use a L-cysteine residue such that maleimide is bonded to the sulphur of the L-cysteine or may use a L-Lysine residue such that the maleimide is bonded to the nitrogen of the L-lysine. In embodiments comprising a maleimide based linker, the peptide may further comprise a C-terminal L-cysteine residue or L-lysine residue that is bonded to the maleimide based linker, such as maleimidocaproyl, maleimidomethyl cyclohexane-1-carboxylate.

In certain embodiments, in peptides according to the eighth aspect of the invention, one of R¹ and R² is —CH₃ and one of R¹ and R² is hydrogen.

Preferably, in peptides according to the eighth aspect of the invention R¹ and R² are hydrogen.

In a ninth aspect, the invention provides a peptide comprising Formula II

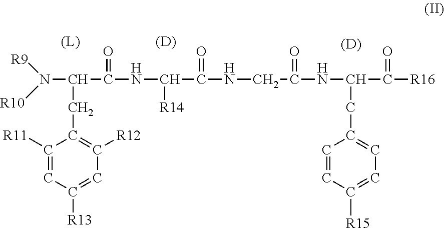

wherein, counting from the N-terminus, the first amino acid residue is an L-amino acid residue and the second and fourth amino acid residues are D-amino acid residues; wherein
R⁹ is hydrogen or a bio-reversible moiety optionally comprising a sugar moiety;
R¹⁰ is hydrogen or a bio-reversible moiety optionally comprising a sugar moiety;
wherein when one of R⁹ or R¹⁰ is a hydrogen and one of R⁹ or R¹⁰ is a bio-reversible moiety, the bio-reversible moiety is preferably —C(═O)OZ₃ or —C(═O)OCH₂OC(═O)Z₄;
   Z₃ is C₁-C₆ alkyl or aryl, preferably Z₁ is —CH₂CH₃;
   Z₄ is C₁-C₆ alkyl or aryl, preferably Z₂ is —CH₃;
wherein R⁹ and R¹⁰ may together form one bio-reversible moiety, wherein preferably the bio-reversible moiety is

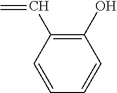

(imine moiety) or ═N═N (azido moiety);
   R¹¹ and R¹² are independently selected from hydrogen or C₁-C₃ alkyl, preferably —CH₃;
   R¹³ is hydrogen, —OH, or a bio-reversible moiety optionally comprising a sugar moiety;
   R¹⁴ is a side chain of an amino acid or C₁-C₆ alkyl, preferably C₁-C₄ alkyl, more preferably —CH(CH₃)₂;
   R¹⁵ is hydrogen, —OH, or a bio-reversible moiety; and
   R¹⁶ is —OH, —O(C₁-C₃ alkyl), —NH₂,

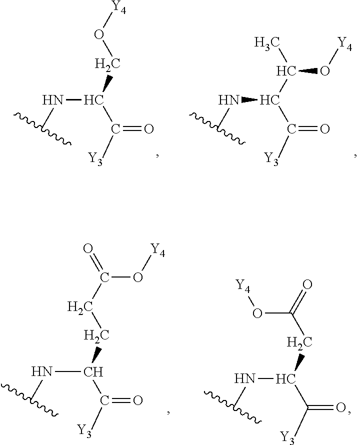

or 1 to about 30 L-amino acid residues;
Y₃ is —OH, —NH₂, or 1 to about 30 L-amino acid residues;
Y₄ is hydrogen or a sugar moiety, preferably a disaccharide moiety; and
wherein when R¹⁶ is 1 to about 30 L-amino acid residues (1) the L-amino acid residues are optionally residues that may be optionally glycosylated with a sugar moiety, preferably a disaccharide moiety, and (2) the C-terminus is optionally amidated.

L-amino acid residues that may be optionally glycosylated include residues that may be N-glycosylated (also referred to as N-linked glycosylation) such as L-asparagine, L-glutamine, L-lysine, L-histidine, and L-arginine; O-glycosylated (also referred to as O-linked glycosylation) such as L-serine, L-threonine, L-tyrosine, L-hydroxylysine, and L-hydroxyproline; S-glycosylated (also referred to as S-linked glycosylation) such as L-cysteine; C-glycosylation (also referred to as C-linked glycosylation) such as L-tryptophan; and Se-glycosylated (also referred to as Se-linked glycosylation) such as L-selenocysteine.

Peptides according to the ninth aspect of the invention include peptides wherein R₁₆ is 1 to about 25 L-amino acid residues, 1 to about 20 L-amino acid residues, 1 to about 15 L-amino acid residues, 1 to about 11 L-amino acid residues, or 1 to about 5 L-amino acid residues.

Peptides according to the ninth aspect of the invention include peptides wherein Y₃ is 1 to about 25 L-amino acid residues, 1 to about 20 L-amino acid residues, 1 to about 15 L-amino acid residues, 1 to about 11 L-amino acid residues, or 1 to about 5 L-amino acid residues.

Peptides according to the ninth aspect of the invention include peptides wherein R₁₆ is 1 L-amino acid residue wherein the L-amino acid residue is preferably a residue that may be optionally glycosylated.

In peptides according to the ninth aspect of the invention comprising a bio-reversible moiety, the bio-reversible moiety may be any known in the art, including, but not limited to, a carbonate, carbamate, imine, ether, ester, and amide moiety. In peptides according to the ninth aspect of the invention comprising a bio-reversible moiety, the bio-reversible moiety optionally comprises a sugar moiety, preferably a disaccharide.

In certain embodiments, in peptides according to the ninth aspect of the invention, one of R⁹ or R¹⁰ is a bio-reversible moiety that is glycosylated with a sugar moiety, preferably a disaccharide moiety.

In certain embodiments, in peptides according to the ninth aspect of the invention, R¹⁴ is a side chain of an amino acid. In certain embodiments, R¹⁴ is a side chain of a threonine.

Preferably, in peptides according to the ninth aspect of the invention R⁹ and R¹⁰ are hydrogen.

In a tenth aspect, the invention provides a peptide comprising Formula II, wherein, counting from the N-terminus, the first amino acid residue is an L-amino acid residue and the second and fourth amino acid residues are D-amino acid residues; wherein

R⁹ is hydrogen or a bio-reversible moiety optionally comprising a sugar moiety;
R¹⁰ is hydrogen or a bio-reversible moiety optionally comprising a sugar moiety;
wherein when one of R⁹ or R¹⁰ is a hydrogen and one of R⁹ or R¹⁰ is a bio-reversible moiety, the bio-reversible moiety is preferably —C(═O)OZ₃ or —C(═O)OCH₂OC(═O)Z₄;
   Z₃ is C₁-C₆ alkyl or aryl, preferably Z₁ is —CH₂CH₃;
   Z₄ is C₁-C₆ alkyl or aryl, preferably Z₂ is —CH₃;
wherein R⁹ and R¹⁰ may together form one bio-reversible moiety, wherein preferably the bio-reversible moiety is

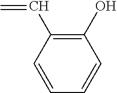

(imine moiety) or ═N═N (azido moiety);
   R¹¹ and R¹² are independently selected from hydrogen or C₁-C₃ alkyl, preferably —CH₃;
   R¹³ is hydrogen, —OH, or a bio-reversible moiety optionally comprising a sugar moiety;
   R¹⁴ is C₁-C₆ alkyl, preferably C₁-C₄ alkyl, more preferably —CH(CH₃)₂;
   R¹⁵ is hydrogen, —OH, or a bio-reversible moiety; and
   R¹⁶ is —OH, —O(C₁-C₃ alkyl), —NH₂,

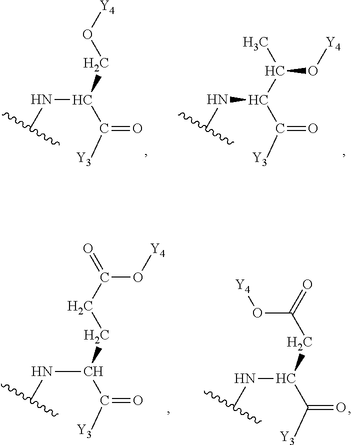

or 1 to about 30 L-amino acid residues;
Y₃ is —OH, —NH₂, or 1 to about 30 L-amino acid residues;
Y₄ is hydrogen or a sugar moiety, preferably a disaccharide moiety; and
wherein when R₁₆ is 1 to about 30 L-amino acid residues (1) the L-amino residues are optionally residues that may be optionally glycosylated with a sugar moiety, preferably a disaccharide moiety, and (2) the C-terminus is optionally amidated.

L-amino acid residues that may be optionally glycosylated include residues that may be N-glycosylated (also referred to as N-linked glycosylation) such as L-asparagine, L-glutamine, L-lysine, L-histidine, and L-arginine; 0-glycosylated (also referred to as O-linked glycosylation) such as L-serine, L-threonine, L-tyrosine, L-hydroxylysine, and L-hydroxyproline; S-glycosylated (also referred to as S-linked glycosylation) such as L-cysteine; C-glycosylation (also referred to as C-linked glycosylation) such as L-tryptophan; and Se-glycosylated (also referred to as Se-linked glycosylation) such as L-selenocysteine.

Peptides according to the tenth aspect of the invention include peptides wherein R₁₆ is 1 to about 25 L-amino acid residues, 1 to about 20 L-amino acid residues, 1 to about 15 L-amino acid residues, 1 to about 11 L-amino acid residues, or 1 to about 5 L-amino acid residues.

Peptides according to the tenth aspect of the invention include peptides wherein Y₃ is 1 to about 25 L-amino acid residues, 1 to about 20 L-amino acid residues, 1 to about 15 L-amino acid residues, 1 to about 11 L-amino acid residues, or 1 to about 5 L-amino acid residues.

Peptides according to the tenth aspect of the invention include peptides wherein R₁₆ is 1 L-amino acid residue wherein the L-amino acid residue is preferably a residue that may be optionally glycosylated.

In peptides according to the tenth aspect of the invention comprising a bio-reversible moiety, the bio-reversible moiety may be any known in the art, including, but not limited to, a carbonate, carbamate, imine, ether, ester, and amide moiety. In peptides according to the tenth aspect of the invention comprising a bio-reversible moiety, the bio-reversible moiety optionally comprises a sugar moiety, preferably a disaccharide.

In certain embodiments, in peptides according to the tenth aspect of the invention, one of R⁹ or R¹⁰ is a bio-reversible moiety that is glycosylated with a sugar moiety, preferably a disaccharide moiety.

Preferably, in peptides according to the tenth aspect of the invention R⁹ and R¹⁰ are hydrogen.

In an eleventh tenth aspect, the invention provides a peptide comprising Formula II wherein, counting from the N-terminus, the first amino acid residue is an L-amino acid residue and the second and fourth amino acid residues are D-amino acid residues; wherein

R⁹ is hydrogen or a bio-reversible moiety optionally comprising a sugar moiety;
R¹⁰ is hydrogen or a bio-reversible moiety optionally comprising a sugar moiety;
wherein when one of R⁹ or R¹⁰ is a hydrogen and one of R⁹ or R¹⁰ is a bio-reversible moiety, the bio-reversible moiety is preferably —C(═O)OZ₃ or —C(═O)OCH₂OC(═O)Z₄;
   Z₃ is C₁-C₆ alkyl or aryl, preferably Z₁ is —CH₂CH₃;
   Z₄ is C₁-C₆ alkyl or aryl, preferably Z₂ is —CH₃;
wherein R⁹ and R¹⁰ may together form one bio-reversible moiety, wherein preferably the bio-reversible moiety is

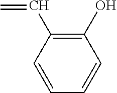

(imine moiety) or ═N═N (azido moiety);
   R¹¹, R¹², and R¹³ are hydrogen;
   R¹⁴ is C₁-C₄ alkyl, preferably —CH(CH₃)₂;

### R¹⁵ is —OH; and

R¹⁶ is —OH, —O(C₁-C₃ alkyl), —NH₂,

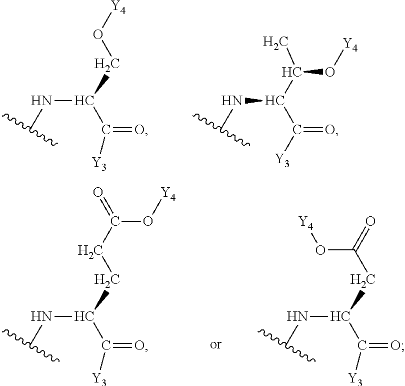

Y₃ is —OH or —NH₂;
Y₄ is hydrogen or a sugar moiety, preferably a disaccharide moiety.

Peptides according to the eleventh aspect of the invention optionally comprise additional L-amino acid residues on the C-terminus of the peptide, wherein said additional L-amino acid residues are optionally glycosylated. Preferably, the peptides of the eleventh aspect of the invention optionally comprise about 5, about 8, about 11, about 12, about 20 or about 26 additional L-amino acid residues on the C-terminus. The peptide optionally comprises additional L-amino acid residues that may optionally be glycosylated, including residues that may be N-glycosylated (also referred to as N-linked glycosylation) such as L-asparagine, L-glutamine, L-lysine, L-histidine, and L-arginine; O-glycosylated (also referred to as O-linked glycosylation) such as L-serine, L-threonine, L-tyrosine, L-hydroxylysine, and L-hydroxyproline; S-glycosylated (also referred to as S-linked glycosylation) such as L-cysteine; C-glycosylation (also referred to as C-linked glycosylation) such as L-tryptophan; and Se-glycosylated (also referred to as Se-linked glycosylation) such as L-selenocysteine. The C-terminus is optionally amidated.

In peptides according to the eleventh aspect of the invention comprising a bio-reversible moiety, the bio-reversible moiety may be any known in the art, including, but not limited to, a carbonate, carbamate, imine, ether, ester, and amide moiety. In peptides according to the eleventh aspect of the invention comprising a bio-reversible moiety, the bio-reversible moiety optionally comprises a sugar moiety, preferably a disaccharide.

In certain embodiments, in peptides according to the eleventh aspect of the invention, one of R⁹ or R¹⁰ is a bio-reversible moiety that is glycosylated with a sugar moiety, preferably a disaccharide moiety.

Preferably, in peptides according to the eleventh aspect of the invention R⁹ and R¹⁰ are hydrogen.

In a twelfth aspect, the invention provides a peptide comprising Formula II wherein, counting from the N-terminus, the first amino acid residue is an L-amino acid residue and the second and fourth amino acid residues are D-amino acid residues; wherein

R⁹ is hydrogen or a bio-reversible moiety optionally comprising a sugar moiety;
R¹⁰ is hydrogen or a bio-reversible moiety optionally comprising a sugar moiety;
wherein when one of R⁹ or R¹⁰ is a hydrogen and one of R⁹ or R¹⁰ is a bio-reversible moiety, the bio-reversible moiety is preferably —C(═O)OZ₃ or —C(═O)OCH₂OC(═O)Z₄;
   Z₃ is C₁-C₆ alkyl or aryl, preferably Z₁ is —CH₂CH₃;
   Z₄ is C₁-C₆ alkyl or aryl, preferably Z₂ is —CH₃;
wherein R⁹ and R¹⁰ may together form one bio-reversible moiety, wherein preferably the bio-reversible moiety is

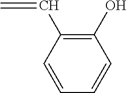

(imine moiety) or ═N═N (azido moiety);
   R¹¹, R¹², and R¹³ are hydrogen;
   R¹⁴ is —CH(CH₃)₂;

### R¹⁵ is —OH; and

R¹⁶ is —OH, —O(C₁-C₃ alkyl), —NH₂,

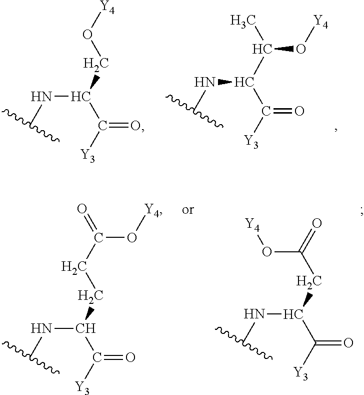

Y₃ is —OH or —NH₂;
Y₄ is hydrogen or a sugar moiety, preferably a disaccharide moiety.

Peptides according to the twelfth aspect of the invention optionally comprise additional L-amino acid residues on the C-terminus of the peptide, wherein said additional L-amino acid residues are optionally glycosylated. Preferably, the peptides of the twelfth aspect of the invention optionally comprise about 5, about 8, about 11, about 12, about 20 or about 26 additional L-amino acid residues on the C-terminus. The peptide optionally comprises additional L-amino acid residues that may optionally be glycosylated, including residues that may be N-glycosylated (also referred to as N-linked glycosylation) such as L-asparagine, L-glutamine, L-lysine, L-histidine, and L-arginine; O-glycosylated (also referred to as O-linked glycosylation) such as L-serine, L-threonine, L-tyrosine, L-hydroxylysine, and L-hydroxyproline; S-glycosylated (also referred to as S-linked glycosylation) such as L-cysteine; C-glycosylation (also referred to as C-linked glycosylation) such as L-tryptophan; and Se-glycosylated (also referred to as Se-linked glycosylation) such as L-selenocysteine. The C-terminus is optionally amidated.

In peptides according to the twelfth aspect of the invention comprising a bio-reversible moiety, the bio-reversible moiety may be any known in the art, including, but not limited to, a carbonate, carbamate, imine, ether, ester, and amide moiety. In peptides according to the twelfth aspect of the invention comprising a bio-reversible moiety, the bio-reversible moiety optionally comprises a sugar moiety, preferably a disaccharide.

In certain embodiments, in peptides according to the twelfth aspect of the invention, one of R⁹ or R¹⁰ is a bio-reversible moiety that is glycosylated with a sugar moiety, preferably a disaccharide moiety.

Preferably, in peptides according to the twelfth aspect of the invention R⁹ and R¹⁰ are hydrogen.

In a thirteenth aspect, the invention provides a peptide comprising Formula II wherein, counting from the N-terminus, the first amino acid residue is an L-amino acid residue and the second and fourth amino acid residues are D-amino acid residues; wherein

R⁹, R¹⁰, R¹¹, R¹², and R¹³ are hydrogen;
R¹⁴ is C₁-C₄ alkyl, preferably —CH(CH₃)₂;

### R¹⁵ is —OH; and

R¹⁶ is —OH, —O(C₁-C₃ alkyl), —NH₂,

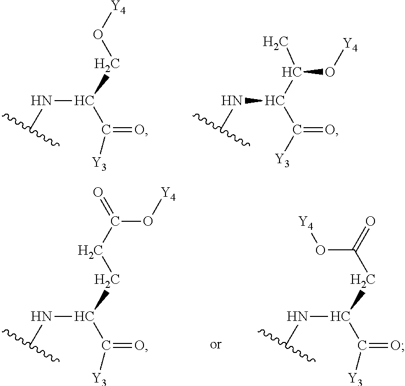

Y₃ is —OH or —NH₂;
Y₄ is hydrogen or a sugar moiety, preferably a disaccharide moiety.

Peptides according to the thirteenth aspect of the invention optionally comprise additional L-amino acid residues on the C-terminus of the peptide, wherein said additional L-amino acid residues are optionally glycosylated. Preferably, the peptides of the thirteenth aspect of the invention optionally comprise about 5, about 8, about 11, about 12, about 20 or about 26 additional L-amino acid residues on the C-terminus. The peptide optionally comprises additional L-amino acid residues that may be optionally glycosylated include residues that may be N-glycosylated (also referred to as N-inked glycosylation) such as L-asparagine, L-glutamine, L-lysine, L-histidine, and L-arginine; O-glycosylated (also referred to as O-linked glycosylation) such as L-serine, L-threonine, L-tyrosine, L-hydroxylysine, and L-hydroxyproline; S-glycosylated (also referred to as S-linked glycosylation) such as L-cysteine; C-glycosylation (also referred to as C-linked glycosylation) such as L-tryptophan; and Se-glycosylated (also referred to as Se-linked glycosylation) such as L-selenocysteine. The C-terminus is optionally amidated.

In a fourteenth aspect, the invention provides a peptide comprising Formula II, wherein, counting from the N-terminus, the first amino acid residue is an L-amino acid residue and the second and fourth amino acid residues are D-amino acid residues; wherein

R⁹ is hydrogen, a single bond, or a —C₁-C₃ alkyl, preferably —CH₃;
R¹⁰ is hydrogen, a single bond, or a —C₁-C₃ alkyl, preferably —CH₃;
R¹¹ and R¹² are independently selected from hydrogen or C₁-C₃ alkyl, preferably —CH₃;
R¹³ is hydrogen, —OH, or —O(C₁-C₃)alkyl;
R¹⁴ is a side chain of an amino acid, preferably a side chain of a threonine residue, or C₁-C₆ alkyl, preferably C₁-C₄ alkyl, more preferably —CH(CH₃)₂;
R¹⁵ is hydrogen, —OH, or a bio-reversible moiety; and
R¹⁶ is —OH, —O(C₁-C₃ alkyl), —NH₂,

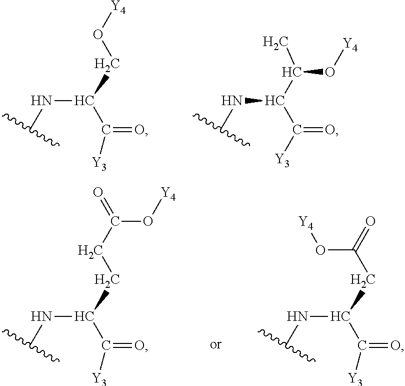

1 to about 30 L-amino acid residues, or a linker;
Y₃ is —OH, —NH₂, or 1 to about 30 L-amino acid residues;
Y₄ is hydrogen or a sugar moiety, preferably a disaccharide moiety;
wherein when R¹⁶ is 1 to about 30 L-amino acid residues (1) the L-amino acid residues are optionally residues that may be optionally glycosylated with a sugar moiety, preferably a disaccharide moiety, and (2) the C-terminus is optionally amidated;
wherein when R¹⁶ is a linker, the linker comprises a sugar moiety, preferably a disaccharide moiety such as lactose, and
wherein when one of R⁹ or R¹⁰ is a single bond, one of R⁹ or R¹⁰ is hydrogen and the single bond is a peptide bond to an L-amino acid residue that may optionally be N-terminally alkylated, preferably singly methylated.

L-amino acid residues that may be optionally glycosylated include residues that may be N-glycosylated (also referred to as N-linked glycosylation) such as L-asparagine, L-glutamine, L-lysine, L-histidine, and L-arginine; O-glycosylated (also referred to as O-linked glycosylation) such as L-serine, L-threonine, L-tyrosine, L-hydroxylysine, and L-hydroxyproline; S-glycosylated (also referred to as S-linked glycosylation) such as L-cysteine; C-glycosylation (also referred to as C-linked glycosylation) such as L-tryptophan; and Se-glycosylated (also referred to as Se-linked glycosylation) such as L-selenocysteine.

Peptides according to the fourteenth aspect of the invention include peptides wherein R₁₆ is 1 to about 25 L-amino acid residues, 1 to about 20 L-amino acid residues, 1 to about 15 L-amino acid residues, 1 to about 11 L-amino acid residues, or 1 to about 5 L-amino acid residues.

Peptides according to the fourteenth aspect of the invention include peptides wherein Y₃ is 1 to about 25 L-amino acid residues, 1 to about 20 L-amino acid residues, 1 to about 15 L-amino acid residues, 1 to about 11 L-amino acid residues, or 1 to about 5 L-amino acid residues.

Peptides according to the fourteenth aspect of the invention include peptides wherein R₁₆ is 1 L-amino acid residue wherein the L-amino acid residue is preferably a residue that may be optionally glycosylated.

In peptides according to the fourteenth aspect of the invention comprising a bio-reversible moiety, the bio-reversible moiety may be any known in the art, including, but not limited to, a carbonate, carbamate, imine, ether, ester, and amide moiety. In peptides according to the fourteenth aspect of the invention comprising a bio-reversible moiety, the bio-reversible moiety optionally comprises a sugar moiety, preferably a disaccharide.

In certain embodiments, in peptides according to the fourteenth aspect of the invention, R¹⁴ is a side chain of an amino acid, preferably a side chain of a threonine residue.

In certain embodiments, in peptides according to the fourteenth aspect of the invention, one of R⁹ and R¹⁰ is —CH₃ and one of R⁹ and R¹⁰ is hydrogen.

In peptides according to the fourteenth aspect of the invention comprising a linker as R¹⁶, the linker is not particularly limited and may be any known in the art. Suitable linkers include amino acid based linkers, including but not limited to single amino acid linkers, such as L-Cysteine, L-lysine, L-Serine, L-threonine, and the like, peptide based linkers including but not limited to L-Valine-L-Citrulline, L-Phe-L-Lys, L-Glutamic acid-L-Valine-L-Citrulline, and the like, amino acid comprising linkers, including but not limited to valine-citrulline-p-aminocarbamate (VC-PABC), and the like, and maleimide based linkers, including but not limited to maleimidocaproyl, maleimidomethyl cyclohexane-1-carboxylate and the like; as well as combinations of such linkers such as maleimidocaproyl-valine-citrulline-p-aminocarbamate, as well as amino and carboxy group containing linkers such as 6-aminohexanoic acid, and the like. The skilled person will appreciate that maleimide based linkers may use a L-cysteine residue such that maleimide is bonded to the sulphur of the L-cysteine or may use a L-Lysine residue such that the maleimide is bonded to the nitrogen of the L-lysine. In embodiments comprising a maleimide based linker, the peptide may further comprise a C-terminal L-cysteine residue or L-lysine residue that is bonded to the maleimide based linker, such as maleimidocaproyl, maleimidomethyl cyclohexane-1-carboxylate.

Preferably, in peptides according to the fourteenth aspect of the invention R⁹ and R¹⁰ are hydrogen.

In a fifteenth aspect, the invention provides an isolated peptide comprising Formula III

X¹—X²—X³—X⁴ (III)

wherein:
X¹ is the N-terminal amino acid residue comprising an N-terminal moiety —NR¹⁷R¹⁸;
X⁴ comprises a C-terminal moiety selected from —C(═O)OH, —C(═O)O(C₁-C₃ alkyl), —C(═O)NH₂,

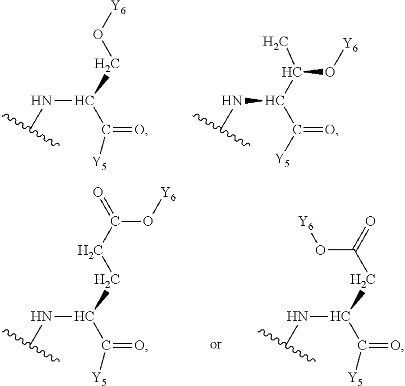

Wherein Y₅ is —OH or —NH₂, and Y₆ is hydrogen or a sugar moiety, preferably a disaccharide moiety;
X¹ is an L-amino acid residue selected from L-tyrosine, 2,6-dimethyl-L-tyrosine, or L-phenylalanine, wherein when X¹ is L-tyrosine or 2,6-dimethyl-L-tyrosine, the residue is optionally O-substituted at the 4-position with a bio-reversible moiety;
X² is a D-amino acid residue, preferably D-alanine, D-valine, D-leucine, or D-isoleucine, more preferably, D-valine;
X³ is glycine or an L-amino acid residue, wherein when X³ is an L-amino acid residue, X³ is preferably L-alanine, L-valine, L-leucine, or L-isoleucine, more preferably L-valine;
X⁴ is a D-amino acid residue selected from D-tyrosine or D-phenylalanine, wherein when X⁴ is D-tyrosine, the residue is optionally O-substituted with a bio-reversible moiety at the 4-position;
R¹⁷ and R¹⁸ are independently selected from hydrogen or a bio-reversible moiety optionally comprising a sugar moiety, or R¹⁷ and R¹⁸ together form a bio-reversible moiety optionally comprising a sugar moiety, and
wherein the peptide is a MOPr agonist.

Peptides according to the fifteenth aspect of the invention optionally comprise additional L-amino acid residues on the C-terminus of the peptide, wherein said additional L-amino acid residues are optionally glycosylated. Preferably, the peptides of the fifteenth aspect of the invention optionally comprise about 5, about 8, about 11, about 12, about 20 or about 26 additional L-amino acid residues on the C-terminus. The peptide optionally comprises additional L-amino acid residues that may be optionally glycosylated include residues that may be N-glycosylated (also referred to as N-linked glycosylation) such as L-asparagine, L-glutamine, L-lysine, L-histidine, and L-arginine; O-glycosylated (also referred to as O-linked glycosylation) such as L-serine, L-threonine, L-tyrosine, L-hydroxylysine, and L-hydroxyproline; S-glycosylated (also referred to as S-linked glycosylation) such as L-cysteine; C-glycosylation (also referred to as C-linked glycosylation) such as L-tryptophan; and Se-glycosylated (also referred to as Se-linked glycosylation) such as L-selenocysteine. The C-terminus is optionally amidated.

In peptides according to the fifteenth aspect of the invention comprising a bio-reversible moiety, the bio-reversible moiety may be any known in the art, including, but not limited to, a carbonate, carbamate, imine, ether, ester, and amide moiety. In peptides according to the fifteenth aspect of the invention comprising a bio-reversible moiety, the bio-reversible moiety optionally comprises a sugar moiety, preferably a disaccharide. In certain embodiments, in peptides according to the fifteenth aspect of the invention, one of R¹⁷ or R¹⁸ is a bio-reversible moiety that is glycosylated with a sugar moiety, preferably a disaccharide moiety. In certain embodiment, in peptides according to the fifteenth aspect of the invention, R¹⁷ and R¹⁸ together form a bio-reversible moiety that is glycosylated with a sugar moiety, preferably a disaccharide moiety. In certain embodiments, in peptides according to the fifteenth aspect of the invention, wherein when X¹ is L-tyrosine or 2,6-dimethyl-L-tyrosine, the residue is O-substituted with a bio-reversible moiety at the 4-position and further the bio-reversible moiety is glycosylated.

In certain embodiments according to the fifteenth aspect of the invention, X² is a D-threonine residue and/or X³ is an L-threonine residue. In certain embodiments X² is a D-threonine residue and X³ is an L-threonine residue. In certain embodiments, X² is a D-threonine residue and X³ is an L-valine residue or X² is a D-valine residue and X³ is an L-threonine residue.

Preferably, in peptides according to the fifteenth aspect of the invention R¹⁷ and R¹⁸ are hydrogen.

In a sixteenth aspect, the invention provides a peptide comprising Formula III, wherein X¹ is the N-terminal amino acid residue comprising an N-terminal moiety —NR¹⁷R¹⁸;

R¹⁷ and R¹⁸ are each hydrogen;
X⁴ comprises a C-terminal moiety selected from —C(═O)OH, —C(═O)O(C₁-C₃ alkyl), —C(═O)NH₂,

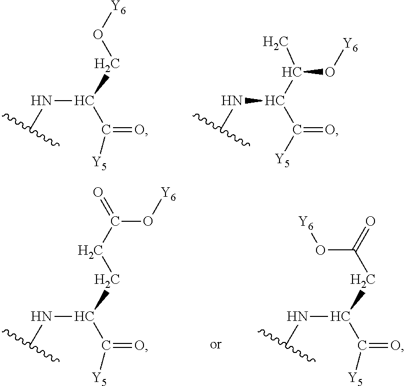

wherein Y₅ is —OH or —NH₂, and Y₆ is hydrogen or a sugar moiety, preferably a disaccharide moiety;
X² is a D-valine residue; and
X³ is a glycine residue or an L-valine residue.

Peptides according to the sixteenth aspect of the invention optionally comprise additional L-amino acid residues on the C-terminus of the peptide, wherein said additional L-amino acid residues are optionally glycosylated. Preferably, the peptides of the sixteenth aspect of the invention optionally comprise about 5, about 8, about 11, about 12, about 20 or about 26 additional L-amino acid residues on the C-terminus. The peptide optionally comprises additional L-amino acid residues that may be optionally glycosylated include residues that may be N-glycosylated (also referred to as N-linked glycosylation) such as L-asparagine, L-glutamine, L-lysine, L-histidine, and L-arginine; O-glycosylated (also referred to as O-linked glycosylation) such as L-serine, L-threonine, L-tyrosine, L-hydroxylysine, and L-hydroxyproline; S-glycosylated (also referred to as S-linked glycosylation) such as L-cysteine; C-glycosylation (also referred to as C-linked glycosylation) such as L-tryptophan; and Se-glycosylated (also referred to as Se-linked glycosylation) such as L-selenocysteine. The C-terminus is optionally amidated.

In a seventeenth aspect, the invention provides an isolated peptide comprising Formula III, wherein:

X¹ is the N-terminal amino acid residue comprising an N-terminal moiety —NR¹⁷R¹⁸;
X⁴ comprises a C-terminal moiety selected from —C(═O)OH, —C(═O)O(C₁-C₃ alkyl), —C(═O)NH₂,

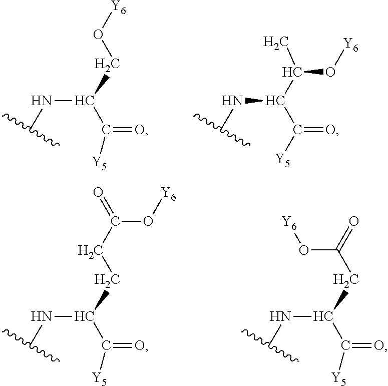

or linker, wherein Y₅ is —OH or —NH₂, and Y₆ is hydrogen or a sugar moiety, preferably a disaccharide moiety;
X¹ is an L-amino acid residue selected from L-tyrosine, 2,6-dimethyl-L-tyrosine, or L-phenylalanine, wherein when X¹ is L-tyrosine or 2,6-dimethyl-L-tyrosine, the residue is optionally O-substituted at the 4-position C₁-C₃ alkyl;
X² is a D-amino acid residue, preferably D-threonine, D-alanine, D-valine, D-leucine, or D-isoleucine, more preferably, D-threonine or D-valine;
X³ is glycine or an L-amino acid residue, wherein when X³ is an L-amino acid residue, X³ is preferably L-threonine, L-alanine, L-valine, L-leucine, or L-isoleucine, more preferably L-threonine or L-valine;
X⁴ is a D-amino acid residue selected from D-tyrosine or D-phenylalanine, wherein when X⁴ is D-tyrosine, the residue is optionally O-substituted with a bio-reversible moiety at the 4-position;
R¹⁷ and R¹⁸ are independently selected from hydrogen, single bond, or —C₁-C₃ alkyl, preferably —CH₃;
wherein when X⁴ comprises a linker, the linker comprises a sugar moiety, preferably a disaccharide moiety such as lactose, and
wherein when one of R¹⁷ or R¹⁸ is a single bond, one of R¹⁷ and R¹⁸ is hydrogen and the single bond is a peptide bond to an L-amino acid residue that may optionally be N-terminally alkylated, preferably singly methylated; and
wherein the peptide is a MOPr agonist.

Peptides according to the seventeenth aspect of the invention optionally comprise additional L-amino acid residues on the C-terminus of the peptide, wherein said additional L-amino acid residues are optionally glycosylated. Preferably, the peptides of the seventeenth aspect of the invention optionally comprise about 5, about 8, about 11, about 12, about 20 or about 26 additional L-amino acid residues on the C-terminus. The peptide optionally comprises additional L-amino acid residues that may be optionally glycosylated include residues that may be N-glycosylated (also referred to as N-linked glycosylation) such as L-asparagine, L-glutamine, L-lysine, L-histidine, and L-arginine; O-glycosylated (also referred to as O-linked glycosylation) such as L-serine, L-threonine, L-tyrosine, L-hydroxylysine, and L-hydroxyproline; S-glycosylated (also referred to as S-linked glycosylation) such as L-cysteine; C-glycosylation (also referred to as C-linked glycosylation) such as L-tryptophan; and Se-glycosylated (also referred to as Se-linked glycosylation) such as L-selenocysteine. The C-terminus is optionally amidated.

In peptides according to the seventeenth aspect of the invention comprising a bio-reversible moiety, the bio-reversible moiety may be any known in the art, including, but not limited to, a carbonate, carbamate, imine, ether, ester, and amide moiety. In peptides according to the seventeenth aspect of the invention comprising a bio-reversible moiety, the bio-reversible moiety optionally comprises a sugar moiety, preferably a disaccharide.

In certain embodiments according to the seventeenth aspect of the invention, X² is a D-threonine residue and/or X³ is an L-threonine residue. In certain embodiments X² is a D-threonine residue and X³ is an L-threonine residue. In certain embodiments, X² is a D-threonine residue and X³ is an L-valine residue or X² is a D-valine residue and X³ is an L-threonine residue.

In peptides according to the seventeenth aspect of the invention, when X⁴ comprises a linker, the linker is not particularly limited and may be any known in the art. Suitable linkers include amino acid based linkers, including but not limited to single amino acid linkers, such as L-Cysteine, L-lysine, L-Serine, L-threonine, and the like, peptide based linkers including but not limited to L-Valine-L-Citrulline, L-Phe-L-Lys, L-Glutamic acid-L-Valine-L-Citrulline, and the like, amino acid comprising linkers, including but not limited to valine-citrulline-p-aminocarbamate (VC-PABC), and the like, and maleimide based linkers, including but not limited to maleimidocaproyl, maleimidomethyl cyclohexane-1-carboxylate and the like; as well as combinations of such linkers such as maleimidocaproyl-valine-citrulline-p-aminocarbamate, as well as amino and carboxy group containing linkers such as 6-aminohexanoic acid, and the like. The skilled person will appreciate that maleimide based linkers may use a L-cysteine residue such that maleimide is bonded to the sulphur of the L-cysteine or may use a L-Lysine residue such that the maleimide is bonded to the nitrogen of the L-lysine. In embodiments comprising a maleimide based linker, the peptide may further comprise a C-terminal L-cysteine residue or L-lysine residue that is bonded to the maleimide based linker, such as maleimidocaproyl, maleimidomethyl cyclohexane-1-carboxylate.

In certain embodiments according to the seventeenth aspect of the invention, one of R¹⁷ and R¹⁸ is hydrogen and one of R¹⁷ and R¹⁸ is a —CH₃.

Preferably, in peptides according to the seventeenth aspect of the invention R¹⁷ and R¹⁸ are hydrogen.

In an eighteenth aspect, the invention provides a peptide comprising Formula IV,

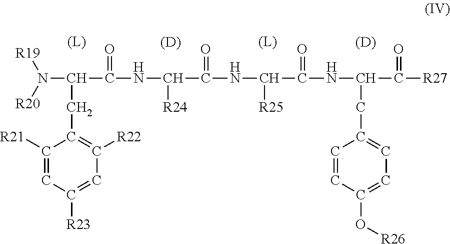

wherein, counting from the N-terminus, the first and third amino acid residues are L-amino acid residues and the second and fourth amino acid residues are D-amino acid residues; wherein
one of R¹⁹ or R²⁰ is hydrogen and one of R¹⁹ or R²⁰ is a bio-reversible moiety that comprises a sugar moiety, preferably a disaccharide moiety, or R¹⁹ and R²⁰ together form a bio-reversible moiety that comprises a sugar moiety, preferably a disaccharide moiety;
R²¹ and R²² are independently selected from hydrogen or C₁-C₃ alkyl, preferably —CH₃;
R²³ is hydrogen, —OH, or a bio-reversible moiety optionally comprising a sugar moiety;
R²⁴ is a side chain of an amino acid or C₁-C₆ alkyl, preferably C₁-C₄ alkyl, more preferably CH(CH₃)₂;
R²⁵ is a side chain of an amino acid or C₁-C₆ alkyl, preferably C₁-C₄ alkyl, more preferably CH(CH₃)₂;
R²⁶ is hydrogen or a bio-reversible moiety optionally comprising a sugar moiety; and
R²⁷ is —OH, —O(C₁-C₃ alkyl), or —NH₂.

In peptides according to the eighteenth aspect, the bio-reversible moiety may be any known in the art, including, but not limited to, a carbonate, carbamate, imine, ether, ester, and amide moiety.

In peptides according to the eighteenth aspect, the sugar moiety comprised by R¹⁹ or R²⁰ is preferably a disaccharide moiety.

In certain embodiments, in peptide according to the eighteenth aspect, R¹⁹ or R²⁰ comprise an additional N-terminal L-amino acid residue(s) that is glycosylated, including residues that may be N-glycosylated (also referred to as N-linked glycosylation) such as L-asparagine, L glutamine, L-lysine, L-histidine, and L-arginine; O-glycosylated (also referred to as O-linked glycosylation) such as L-serine, L-threonine, L-tyrosine, L-hydroxylysine, and L-hydroxyproline; S-glycosylated (also referred to as S-linked glycosylation) such as L-cysteine; C-glycosylation (also referred to as C-linked glycosylation) such as L-tryptophan; and Se-glycosylated (also referred to as Se-linked glycosylation) such as L-selenocysteine. In certain embodiments, one of R¹⁹ or R²⁰ is

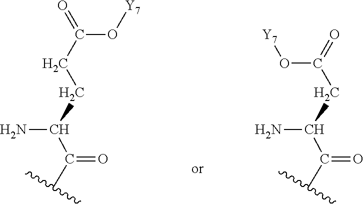

wherein Y₇ is the sugar moiety, preferably a disaccharide moiety.

Peptides according to the eighteenth aspect of the invention optionally comprise additional L-amino acid residues on the C-terminus of the peptide, wherein said additional L-amino acid residues are optionally glycosylated. Preferably, the peptides of the eighteenth aspect of the invention optionally comprise about 5, about 8, about 11, about 12, about 20 or about 26 additional L-amino acid residues on the C-terminus. The C-terminus is optionally amidated.

Examples of a peptide of the invention include the following:

L-Phe-D-Val-L-Val-D-Phe (peptide 1a, Bilaid A) (SEQ ID NO: 1);
L-Phe-D-Val-L-Val-D-Phe-NH₂ (peptide 1e) (SEQ ID NO: 7);
L-Tyr-D-Val-L-Val-D-Phe (peptide 3a, Bilaid C) (SEQ ID NO: 3);
L-Tyr-D-Val-L-Val-D-Phe-NH₂ (peptide 3b) (SEQ ID NO: 16);
2,6-dimethyl-L-tyrosine-D-Val-L-Val-D-Phe-NH₂ (peptide 3c; Bilorphin) (SEQ ID NO: 17);
2,6-dimethyl-L-tyrosine-D-Val-L-Val-D-Phe-L-Ser(β-Lac)-NH₂ (peptide 3g; Bilactorphin) (SEQ ID NO: 21);
L-Phe-D-Val-Gly-D-Tyr-NH₂ (peptide 2d) (SEQ ID NO: 13);
2,6-dimethyl-L-tyrosine-D-Val-L-Val-D-Phe-OCH₂CH₃ (peptide 4) (SEQ ID NO: 23);
2,6-dimethyl-L-tyrosine-D-Val-L-Val-D-Phe-L-Pro-L-Asn-L-Leu-L-Aa-L-Glu-L-Lys-L-AIa-L-Leu-L-Lys-L-Ser-L-Leu-NH₂ (peptide 11) (SEQ ID NO: 30);
2,6-dimethyl-L-tyrosine-D-Val-L-Val-D-Phe-NH₂ wherein the N-terminus is substituted with the bio-reversible moiety —C(═O)OCH₂OC(═O)CH₃ (peptide 10) (SEQ ID NO: 29);
2,6-dimethyl-L-tyrosine-D-Val-L-Val-D-Phe-NH₂ wherein the N-terminus is substituted with the bio-reversible moiety

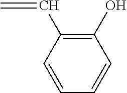

(peptide 8) (SEQ ID NO: 27);
2,6-dimethyl-L-tyrosine-D-Val-L-Val-D-Phe-NH₂ wherein the hydroxy group on 2,6-dimethyl-L-tyrosine is substituted with the bio-reversible moiety —C(═O)CH₃ (peptide 5) (SEQ ID NO: 24);
2,6-dimethyl-L-tyrosine-D-Val-L-Val-D-Phe-OCH₂CH₃ wherein the hydroxy group on 2,6-dimethyl-L-tyrosine is substituted with the bio-reversible moiety —C(═O)CH₃ (peptide 6) (SEQ ID NO: 25);
2,6-dimethyl-L-tyrosine-D-Val-L-Val-D-Phe-NH₂ wherein the N-terminus is substituted with the bio-reversible moiety —C(═O)OCH₂CH₃ (peptide 7) (SEQ ID NO: 26); and
2,6-dimethyl-L-tyrosine-D-Val-L-Val-D-Phe-NH₂ wherein the N-terminus is substituted with the bio-reversible moiety, ═N═N, to form an N-terminal azido group (peptide 9) (SEQ ID NO: 28).

Further Examples of a peptide of the invention include the following:

L-AA-L-Tyr-D-Val-L-Val-D-Phe-linker-sugar moiety;
L-AA-L-Tyr-D-Thr-L-Thr-D-Phe-linker-sugar moiety;
L-AA-L-Dmt-D-Val-L-Val-D-Phe-linker-sugar moiety;
L-AA-L-Dmt-D-Thr-L-Thr-D-Phe-linker-sugar moiety;
wherein L-AA is any L-amino acid residue optionally comprising at least one N-terminal —CH₃;
wherein the hydroxy group of L-Tyr or L-Dmt is optionally alkylated; and
wherein the linker is preferably L-Ser or L-Thr.

L-Phe-D-Val-L-Val-D-Phe (peptide 1a, Bilaid A) is

(1) a peptide of Formula I wherein R¹, R², R³, R⁴, and R⁵ are hydrogen, R⁶ and R⁷ are —CH(CH₃)₂, and R⁸ is —OH; or
(2) a peptide of Formula III wherein X¹ is the N-terminal amino acid, X¹ is an L-phenylalanine residue, X² is a D-valine residue, X³ is an L-valine residue, X⁴ is a D-phenylalanine residue with a C-terminal —C(═O)OH moiety, X¹ has an N-terminal —NR¹⁷R¹⁸ moiety wherein R¹⁷ and R¹⁸ are each hydrogen.

L-Phe-D-Val-L-Val-D-Phe-NH₂ (peptide 1e) is

(1) a peptide of Formula I wherein R¹, R², R³, R⁴, and R⁵ are hydrogen, R⁶ and R⁷ are —CH(CH₃)₂, and R⁸ is —NH₂; or
(2) a peptide of Formula III wherein X¹ is the N-terminal amino acid, X¹ is an L-phenylalanine residue, X² is a D-valine residue, X³ is an L-valine residue, X⁴ is a D-phenylalanine residue with a C-terminal —C(═O)NH₂ moiety, X¹ has an N-terminal —NR¹⁷R¹⁸ moiety wherein R¹⁷ and R¹⁸ are each hydrogen.

L-Tyr-D-Val-L-Val-D-Phe (peptide 3a, Bilaid C) is

(1) a peptide of Formula I wherein R¹, R², R³, and R⁴ are hydrogen, R⁵ is —OH, R⁶ and R⁷ are —CH(CH₃)₂, and R⁸ is —OH; or
(2) a peptide of Formula III wherein X¹ is the N-terminal amino acid, X¹ is an L-tyrosine residue, X² is a D-valine residue, X³ is an L-valine residue, X⁴ is a D-phenylalanine residue with a C-terminal —C(═O)OH moiety, X¹ has an N-terminal —NR¹⁷R¹⁸ moiety wherein R¹⁷ and R¹⁸ are each hydrogen.

L-Tyr-D-Val-L-Val-D-Phe-NH₂ (peptide 3b) is

(1) a peptide of Formula I wherein R¹, R², R³, and R⁴ are hydrogen, R⁵ is —OH, R⁶ and R⁷ are —CH(CH₃)₂, and R₈ is —NH₂; or
(2) a peptide of Formula III wherein X¹ is the N-terminal amino acid, X¹ is an L-tyrosine residue, X² is a D-valine residue, X³ is an L-valine residue, X⁴ is a D-phenylalanine residue with a C-terminal —C(═O)NH₂ moiety, X¹ has an N-terminal —NR¹⁷R¹⁸ moiety wherein R¹⁷ and R¹⁸ are each hydrogen.

2,6-dimethyl-L-tyrosine-D-Val-L-Val-D-Phe-NH₂ (peptide 3c; Bilorphin) is

(1) a peptide of Formula I wherein R¹ and R² are hydrogen, R³ and R⁴ are —CH₃, R⁵ is —OH, R⁶ and R₇ are —CH(CH₃)₂, and R⁸ is —NH₂; or
(2) a peptide of Formula III wherein X¹ is the N-terminal amino acid, X¹ is an 2,6-dimethyl-L-tyrosine residue, X² is a D-valine residue, X³ is an L-valine residue, X⁴ is a D-phenylalanine residue with a C-terminal —C(═O)NH₂ moiety, X¹ has an N-terminal —NR¹⁷R¹⁸ moiety wherein R¹⁷ and R¹⁸ are each hydrogen.

2,6-dimethyl-L-tyrosine-D-Val-L-Val-D-Phe-L-Ser(β-Lac)-NH₂ (peptide 3g; Bilactorphin) is

(1) a peptide of Formula I wherein R¹ and R² are hydrogen, R³ and R⁴ are —CH₃, R⁵ is —OH, R⁶ and R⁷ are —CH(CH₃)₂, and R⁸ is

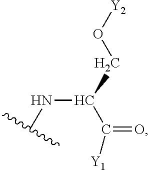

Y₁ is —NH₂ and Y₂ is a sugar moiety, which is disaccharide lactose moiety, wherein the lactose moiety is attached through a beta linkage; or
(2) a peptide of Formula III wherein X¹ is the N-terminal amino acid, X¹ is an 2,6-dimethyl-L-tyrosine residue, X² is a D-valine residue, X³ is an L-valine residue, X⁴ is a D-phenylalanine residue, X¹ has an N-terminal —NR¹⁷R¹⁸ moiety wherein R¹⁷ and R¹⁸ are each hydrogen, and X⁴ comprises a C-terminal

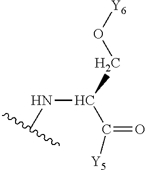

wherein Y₅ is —NH₂ and Y₆ is a sugar moiety, which is a disaccharide lactose moiety, wherein the lactose moiety is attached through a beta linkage.

L-Phe-D-Val-Gly-D-Tyr-NH₂ (peptide 2d) is

(1) a peptide of Formula II wherein R⁹, R¹⁰, R¹¹, R¹², and R¹³ are hydrogen; R¹⁴ is —CH(CH₃)₂; R¹⁵ is —OH; and R¹⁶—NH₂; or
(2) a peptide of Formula III wherein X¹ is the N-terminal amino acid, X¹ is an L-phenylalanine residue, X² is a D-valine residue, X³ is glycine residue, X⁴ is a D-tyrosine residue with a C-terminal —C(═O)NH₂ moiety, X¹ has an N-terminal —NR¹⁷R¹⁸ moiety wherein R¹⁷ and R¹⁸ are each hydrogen.

2,6-dimethyl-L-tyrosine-D-Val-L-Val-D-Phe-OCH₂CH₃ (peptide 4) is

(1) a peptide of Formula I wherein R¹ and R² are hydrogen, R³ and R⁴ are —CH₃, R⁵ is —OH, R⁶ and R⁷ are —CH(CH₃)₂, and R⁸ is —O(CH₂CH₃); or
(2) a peptide of Formula III wherein X¹ is the N-terminal amino acid, X¹ is an 2,6-dimethyl-L-tyrosine residue, X² is a D-valine residue, X³ is an L-valine residue, X⁴ is a D-phenylalanine residue with a C-terminal —C(═O)OCH₂CH₃ moiety, X¹ has an N-terminal —NR¹⁷R¹⁸ moiety wherein R¹⁷ and R¹⁸ are each hydrogen.

2,6-dimethyl-L-tyrosine-D-Val-L-Val-D-Phe-L-Pro-L-Asn-L-Leu-L-Ala-L-Glu-L-Lys-L-Ala-L-Leu-L-Lys-L-Ser-L-Leu-NH₂ (peptide 11) is

(1) a peptide of Formula I wherein R¹ and R² are hydrogen, R³ and R⁴ are —CH₃, R⁵ is —OH, R⁶ and R⁷ are —CH(CH₃)₂, and R⁸ is eleven additional L-amino acid residues and the C-terminus is amidated; or
(2) a peptide of Formula III wherein X¹ is the N-terminal amino acid, X¹ is an 2,6-dimethyl-L-tyrosine residue, X² is a D-valine residue, X³ is an L-valine residue, X⁴ is a D-phenylalanine residue, X¹ has an N-terminal —NR¹⁷R¹⁸ moiety wherein R¹⁷ and R¹⁸ are each hydrogen, and the peptide contains 11 additional L-amino acid residues and the C-terminus is amidated.

2,6-dimethyl-L-tyrosine-D-Val-L-Val-D-Phe-NH₂ wherein the N-terminus is substituted with the bio-reversible moiety —C(═O)OCH₂OC(═O)CH₃ (peptide 10) is

(1) a peptide of Formula I wherein R³ and R⁴ are —CH₃, R₅ is —OH, R⁶ and R⁷ are —CH(CH₃)₂, and R⁸ is —NH₂ and one of R¹ or R² is hydrogen and one of R¹ or R² is the bio-reversible moiety —C(═O)OCH₂OC(═O)CH₃; or
(2) a peptide of Formula III wherein X¹ is the N-terminal amino acid, X¹ is an 2,6-dimethyl-L-tyrosine residue, X² is a D-valine residue, X³ is an L-valine residue, X⁴ is a D-phenylalanine residue with a C-terminus —C(═O)NH₂ moiety, X¹ has an N-terminal —NR¹⁷R¹⁸ moiety wherein on of R¹⁷ or R¹⁸ is hydrogen and one of R¹⁷ or R¹⁸ is the bio-reversible moiety —C(═O)OCH₂OC(═O)CH₃.

2,6-dimethyl-L-tyrosine-D-Val-L-Val-D-Phe-NH₂ wherein the N-terminus is substituted with the bio-reversible moiety

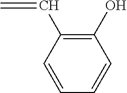

(peptide 8) is
(1) a peptide of Formula I wherein R³ and R⁴ are —CH₃, R₅ is —OH, R⁶ and R⁷ are —CH(CH₃)₂, and R⁸ is —NH₂ and R¹ and R² together form the bio-reversible moiety

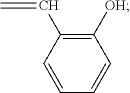

or
(2) a peptide of Formula III wherein X¹ is the N-terminal amino acid, X¹ is an 2,6-dimethyl-L-tyrosine residue, X² is a D-valine residue, X³ is an L-valine residue, X⁴ is a D-phenylalanine residue with a C-terminus —C(═O)NH₂ moiety, X¹ has an N-terminal —NR¹⁷R¹⁸ moiety wherein R¹⁷ and R¹⁸ together form the bio-reversible moiety

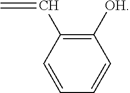

2,6-dimethyl-L-tyrosine-D-Val-L-Val-D-Phe-NH₂ wherein the hydroxy group on 2,6-dimethyl-L-tyrosine is substituted with the bio-reversible moiety —C(═O)CH₃ (peptide 5) is

(1) a peptide of Formula I wherein R¹ and R² are hydrogen, R³ and R⁴ are —CH₃, R⁵ is the bio-reversible moiety —OC(═O)CH₃, R⁶ and R⁷ are —CH(CH₃)₂, and R⁸ is —NH₂; or
(2) a peptide of Formula III wherein X¹ is the N-terminal amino acid, X¹ is an 2,6-dimethyl-L-tyrosine residue, wherein the 4-hydroxyl is substituted with the bio-reversible moiety —C(═O)CH₃; X² is a D-valine residue, X³ is an L-valine residue, X⁴ is a D-phenylalanine residue with a C-terminus —C(═O)NH₂ moiety, X¹ has an N-terminal —NR¹⁷R¹⁸ moiety wherein R¹⁷ and R¹⁸ are hydrogen.

2,6-dimethyl-L-tyrosine-D-Val-L-Val-D-Phe-OCH₂CH₃ wherein the hydroxy group on 2,6-dimethyl-L-tyrosine is substituted with the bio-reversible moiety —C(═O)CH₃ (peptide 6) is

(1) a peptide of Formula I wherein R¹ and R² are hydrogen, R³ and R⁴ are —CH₃, R⁵ is the bio-reversible moiety —OC(═O)CH₃, R⁶ and R⁷ are —CH(CH₃)₂, and R⁸ is —OCH₂CH₃; or
(2) a peptide of Formula III wherein X¹ is the N-terminal amino acid, X¹ is an 2,6-dimethyl-L-tyrosine residue, wherein the 4-hydroxyl is substituted with the bio-reversible moiety —C(═O)CH₃, X² is a D-valine residue, X³ is an L-valine residue, X⁴ is a D-phenylalanine residue with a C-terminus —OCH₂CH₃ moiety, X¹ has an N-terminal —NR¹⁷R¹⁸ moiety wherein R¹⁷ and R¹⁸ are hydrogen.

2,6-dimethyl-L-tyrosine-D-Val-L-Val-D-Phe-NH₂ wherein the N-terminus is substituted with the bio-reversible moiety —C(═O)OCH₂CH₃ (peptide 7) is

(1) a peptide of Formula I wherein R³ and R⁴ are —CH₃, R⁵ is —OH, R⁶ and R⁷ are —CH(CH₃)₂, and R⁸ is —NH₂ and one of R¹ or R² is hydrogen and one of R¹ or R² is the bio-reversible moiety —C(═O)OCH₂CH₃; or
(2) a peptide of Formula III wherein X¹ is the N-terminal amino acid, X¹ is an 2,6-dimethyl-L-tyrosine residue, X² is a D-valine residue, X³ is an L-valine residue, X⁴ is a D-phenylalanine residue with a C-terminus —NH₂ moiety, X¹ has an N-terminal —NR¹⁷R¹⁸ moiety wherein one of R¹⁷ or R¹⁸ are hydrogen and one of R¹⁷ or R¹⁸ is the bio-reversible moiety —C(═O)OCH₂CH.

2,6-dimethyl-L-tyrosine-D-Val-L-Val-D-Phe-NH₂ wherein the N-terminus is substituted with the bio-reversible moiety, ═N═N, to form an N-terminal azido group, (peptide 9) is

(1) a peptide of Formula I wherein R³ and R⁴ are —CH₃, R⁵ is —OH, R⁶ and R⁷ are —CH(CH₃)₂, and R⁸ is —NH₂ and R¹ and R² together form the bio-reversible moiety ═N═N, to form an N-terminal azido group;
(2) a peptide of Formula III wherein X¹ is the N-terminal amino acid, X¹ is an 2,6-dimethyl-L-tyrosine residue, X² is a D-valine residue, X³ is an L-valine residue, X⁴ is a D-phenylalanine residue with a C-terminus —NH₂ moiety, X¹ has an N-terminal —NR¹⁷R¹⁸ moiety wherein R¹⁷ and R¹⁸ together form the bio-reversible moiety ═N═N, to form an N-terminal azido group.

The methods by which the peptides of the invention are produced are not particularly limited and may be any method known in the art. The peptides of the invention may be synthesised using well-known solution-phase techniques or solid-phase methods. The peptides of the invention may be synthesised using Fmoc chemistry wherein the N-terminus of the amino acid residue is protected with Fluorenylmethyloxycarbonyl (Fmoc) protecting group. Methods known in the art, include but are not limited to, methods described in Schnolzer et al. Int J Pept Prot Res (1992) 40: 180-193 and Alewood et al. Methods in Enzymology (1997) 289: 14-29, both of which are incorporated by reference.

The peptides of the invention may be glycosylated with a sugar moiety, for example a monosaccharide, disaccharide, or trisaccharide. Preferably, the sugar moiety is a disaccharide. Suitable methods for linking a sugar moiety to a peptide of the invention are well known in the art. Preferably, the sugar moiety is linked with a beta linkage. While it is not critical whether the sugar moiety is first attached to an amino acid residue, which is then incorporated into a peptide of the invention, or if the amino acids are first assembled into a peptide of the invention, which is then glycosylated. The usual practice has been to glycosylate an amino acid reside and then incorporate that glycosylated amino acid residue into the peptide. Peptides of the invention may comprise one or more amino acid residues with N-glycosylation, O-glycosylation, S-glycosylation, C-glycosylation, or Se-glycosylation. N-linked glycosylation includes glycosylation of amino acid residues such as L-asparagine, L-glutamine, L-lysine, L-histidine, and L-arginine. O-linked glycosylation includes glycosylation of amino acid residues such as L-serine, L-threonine, L-tyrosine, L-hydroxylysine, and L-hydroxyproline. S-linked glycosylation includes glycosylation of amino acid residues such as L-cysteine. C-linked glycosylation includes glycosylation of amino acid residues such as L-tryptophan; and Se-linked glycosylation includes glycosylation of amino acid residues such as L-selenocysteine. Preferably, the peptide of the invention comprises O-linked glycosylation, preferably O-linked glycosylation of an L-serine residue.

In one embodiment of the invention, a peptide of the invention is glycosylated with a monosaccharide moiety. Suitable monosaccharides for glycosylation of the peptides of the invention include, but are not limited to, dihydroxyacetone, glyceraldehydes, aldotriose, erythrulose, erythrose, threose, ribulose, psicose, xylose, glucose (Glc), fructose, mannose, galactose, fucose, ribose, tagatose, arabinose, rhamnose, sedoheptalose and nonoses such as neuraminic acid, sialic acid. Glucose is a preferred monosaccharide.

In one embodiment of the invention, a peptide of the invention is glycosylated with a trisaccharide moiety. Suitable trisaccharides for glycosylation of the peptides of the invention include, but are not limited to, maltotriose and raffinose.

In a preferred embodiment of the invention, a peptide of the invention is glycosylated with a disaccharide moiety. Suitable disaccharides for glycosylation of the peptides of the invention include, but are not limited to sucrose, trehalose, saccharose, maltose, lactose (Lac), cellobiose, gentibiose, isomaltose, melibiose, and primeveose. Preferred disaccharides for glycosylation of a peptide of the invention include lactose and melibiose. Most preferably, the disaccharide is lactose.

Peptides of the invention are MOPr agonists. A peptide MOPr agonist is a peptide that selectively binds to and activates the MOPr, i.e. it stimulates G-protein or other second messenger activity when bound. Peptides may be identified as agonists of MOPr by screening for inhibition of forskolin induced cAMP formation. The forskolin inhibition assay may be carried out in any suitable cell line, including, but not limited to HEK cells, expressing MOPr, preferably a mammalian MOPr such as murine MOPr, more preferably human MOPr (hMOPr). Other methods of assaying for MOPr agonist activity are known in the art, including, but not limited to, simulation of 35S GTP-gamma-S binding in cells expressing MOPr (e.g. McPherson J et al (2010) Molecular Pharmacology 78: 756-766), inhibition of voltage-gated calcium channel currents in cells expressing MOPr (e.g. Borgland S L et al (2003) J Biol Chem 278:18776-18784) or activation of GIRK type potassium currents in cells expressing MOPr (e.g. Yousuf A et al (2015) Molecular Pharmacology 88: 825-835). All references incorporated by reference. Preferably, a peptide of the invention exhibits a capacity to increase inhibition of cAMP formation in comparison to vehicle at a concentration of about 10 μM in a forskolin inhibition assay using hMOPr or activates GIRK currents in cells expressing MOPr.

Peptides of the invention may also be screened for MOPr agonist activity in competitive binding assays using a known MOPr agonist, preferably [³H]DAMGO ([D-Ala², N-MePhe⁴, Gly⁵-ol]-enkephalin). Competitive MOPr binding may be determined using a filtration separation followed by liquid scintillation counting procedure after incubation of membranes prepared from human recombinant MOPr expressed in HEK-293 cells (Human Embryonic Kidney cell line) with [³H]DAMGO (0.5 nM) plus various concentrations of unlabelled peptide under suitable conditions, for example for 120 minutes at 22° C. The specific ligand binding to the receptors may be defined as the difference between the total binding and the nonspecific binding determined in the presence of an excess of an unlabelled opioid ligand, eg naloxone (10 μM). The results may be expressed as a percent of control specific binding ((measured specific binding/control specific binding)×100) obtained in the presence of unlabelled peptides of interest. The IC₅₀ values (concentration causing a half-maximal inhibition of control specific binding) and Hill coefficients (nH) may be determined by non-linear regression analysis of the competition curves generated with mean replicate values using Hill equation curve fitting (Y=D+[(A−D)/(1+(C/C₅₀nH)], where Y=specific binding, D=minimum specific binding, A=maximum specific binding, C=compound concentration, C₅₀=IC₅₀, and nH=slope factor). Preferably, a peptide of the invention exhibits a Kᵢ for MOPr of less than about 5 μM, less than about 3.5 μM, or less than about 1 μM. More preferably, a peptide of the invention exhibits a Kᵢ of less than about 0.8 μM, less than about 0.5 μM, or less than about 0.3 μM.

Similarly inhibition of binding to human recombinant DOPr (hDOPr) expressed in CHO (Chines Hamster Ovary) cell line may be assessed using incubation in [3H]DADLE (0.5 nM) for 120 min at 22° C. Similarly binding to human recombinant KOPr (hKOPr) expressed in CHO (Chines Hamster Ovary) cell line was performed using incubation in [³H]U69593 (2 nM) for 60 min at 22° C.

Peptides of the invention may be screened for their alibility to direct biased G-protein signalling. MOPr C-terminal phosphorylation, β-arrestin recruitment and internalisation are thought to contribute to on-target opioid analgesic side effects so that G-protein biased opioids that avoid β-arrestin signalling may show an improved side effect profile. Preferably, a peptide of the invention exhibits a lower induction of C-terminal phosphorylation of MOPr than morphine. Preferably, a peptide of the invention exhibits a lower induction of β-arrestin recruitment than morphine. Preferably, a peptide of the invention exhibits a lower induction of MOPr internalisation than morphine. More preferably, a peptide of the invention exhibits at least two of the following: a lower induction of C-terminal phosphorylation of MOPr than morphine, a lower induction of β-arrestin recruitment than morphine, and a lower induction of MOPr internalisation than morphine. Most preferably, a peptide of the invention exhibits a lower induction C-terminal phosphorylation of MOPr than morphine, a lower induction of β-arrestin recruitment than morphine, and a lower induction of MOPr internalisation than morphine.

Agonist-induced phosphorylation of serine 375 (Ser375) of MOPr drives β-arrestin recruitment and internalisation (Williams et al., Pharmacol Rev. (2013) 65(1):223-54). Assays to determine induction of phosphorylation of serine 375 (Ser375) of MOPr by a peptide of the invention are not particularly limited and may be determined by any method known in the art. For example, the ability of a peptide of the invention to induce phosphorylation of serine 375 (Ser375) of MOPr can be assessed in an assay using a Ser375-phosphosite specific antibody such as that as described in Just et al “Molecular Pharmacology (2013) 83(3): 633-639, which is incorporated herein by reference. Morphine is known to induce weak phosphorylation of MOPr at Ser375 and may be used for comparative purposes to assess the phosphorylation of MOPr at Ser375 induced by a peptide of the invention. Met-enkephalin and endomorphin 2 are known to induce phosphorylation of MOPr at Ser375 more strongly than morphine and each may be used independently for comparative purposes to assess phosphorylation of MOPr at Ser375 induced by a peptide of the invention. Oliceridine (TRV130) is an established, small molecule, G-protein biased MOPr agonist and may be used for comparative purposes to assess phosphorylation of MOPr at Ser375 induced by a peptide of the invention.

Assays to determine the effect of a peptide of the invention on β-arrestin recruitment by MOPr activation are not particularly limited and may be determined by any method known in the art. For example, the effect of a peptide of the invention on β-arrestin recruitment can be assessed using MOPr-luciferease and β-arrestin 2-YFP constructs in a bioluminescence resonance energy transfer (BRET) assay. Commercially available kits for determining β-arrestin recruitment may also be used. Morphine is known to weakly induce β-arrestin recruitment and may be used as for comparative purposes to assess the 3-arrestin recruitment induced by a peptide of the invention. Met-enkephalin and endomorphin 2 are known to induce 3-arrestin recruitment more strongly than morphine and each may be used independently for comparative purposes to assess 3-arrestin recruitment induced by a peptide of the invention. Oliceridine (TRV130) is an established, small molecule, G-protein biased MOPr agonist and may be used for comparative purposes to assess 3-arrestin recruitment by a peptide of the invention.

Assays to determine the effect of a peptide of the invention on MOPr internalisation are not particularly limited and can be determined by any method known in the art. For example, the effect of a peptide of the invention on MOPr internalisation can be assessed immunocytochemically. For example, cells expressing MOPr are prelabeled with a primary antibody and then stimulated with a peptide of the invention. The changes in surface expression of the MOPr after stimulation are determined by comparison to a corresponding control that has not been treated with a peptide of the invention. Morphine is known to induce weak MOPr internalisation and may be used as for comparative purposes to assess MOPr internalisation induced by a peptide of the invention. Met-enkephalin and endomorphin 2 are known to induce MOPr internalisation more strongly than morphine and each may be used independently for comparative purposes to assess MOPr internalisation induced by a peptide of the invention. Oliceridine (TRV130) is an established, small molecule, G-protein biased MOPr agonist and may be used for comparative purposes to assess MOPr internalisation induced by a peptide of the invention.

Assays to determine the ability to a peptide of the invention to penetrate the central nervous system (also referred to as crossing the blood brain barrier (BBB)) are not particularly limited and may be determined by any method known in the art. For example, a method to assess the ability of a peptide to cross the blood brain barrier (BBB) is to compare injection of a peptide of the invention peripherally (e.g. subcutaneously) and intrathecally in an in vivo model of analgesia assessment may be used. In vitro methods of assessing ability to cross the blood brain barrier (BBB) are known and include, but are not limited to, methods using semipermeable chambers with single brain epithelial cell layers and others. (Wilhelm I et al Molecular Pharmaceutics (2014) 11(7):1949-63, incorporated by reference). In one embodiment, a peptide of the invention crosses the blood brain barrier.

Assessment of in vivo analgesia effect of a peptide of the invention are not particularly limited and may be determined by any method known in the art. Known methods to assess the analgesia effect include tail flick test and hotplate test wherein the pain response of an animal to heat.

The peptides of the invention may be formulated as a pharmaceutical composition comprising a peptide of the invention and at least one pharmaceutically acceptable excipient.

Preferably, the invention is directed toward the use of the peptides of the invention in medicine. The invention includes the peptides of the invention for use in a medicament.

In one embodiment, the invention is directed toward the treatment of pain, including the reduction, amelioration, or suppression of pain, in the broadest sense. In certain embodiments, the invention provides a method of treating pain comprising administering to a subject a peptide of the invention or a pharmaceutical composition comprising a peptide of the invention. In certain embodiments, the invention provides a method of treating pain comprising administering a therapeutically effective amount of a peptide of the invention or a pharmaceutical composition comprising a peptide of the invention to a subject in need thereof. In certain embodiments, the invention provides use of a peptide of the invention or a pharmaceutical composition comprising a peptide of the invention for the manufacture of a medicament for treating pain. In certain embodiments, the invention provides a peptide of the invention or a pharmaceutical composition comprising a peptide of the invention for use in a method of treating pain.

The pain that may be treated with a peptide of the invention or a pharmaceutical composition comprising a peptide of the invention may be any type of pain including, but not limited to, post-operative pain, pain associated with nerve damage, pain associated with bone fracture, pain associated with a burn, pain associated with a wound, short-term, long term, intermittent or persistent, somatic pain, visceral pain, or neuropathic pain. In certain embodiments, the pain that may be treated with a peptide of the invention or a pharmaceutical composition comprising a peptide of the invention may be post-operative pain, pain associated with nerve damage, pain associated with bone fracture, pain associated with a burn, or pain associated with a wound. In certain embodiments, the pain that may be treated with a peptide of the invention or a pharmaceutical composition comprising a peptide of the invention may be short-term, long term, intermittent or persistent, somatic pain, visceral pain, or neuropathic pain.

In certain embodiments, the peptides of the invention are directed toward methods of delivering analgesia. In certain embodiments, the invention provides a method of delivering analgesia comprising administering to a subject a peptide of the invention or a pharmaceutical composition comprising a peptide of the invention. In certain embodiments, the invention provides a method of delivering analgesia comprising administering a therapeutically effective amount of a peptide of the invention or a pharmaceutical composition comprising a peptide of the invention to a subject in need thereof. In certain embodiments, the invention provides use of a peptide of the invention or a pharmaceutical composition comprising a peptide of the invention for the manufacture of a medicament for delivering analgesia. In certain embodiments, the invention provides a peptide of the invention or a pharmaceutical composition comprising a peptide of the invention for use in a method of delivering analgesia.

“Adverse side effect” refers to a medically undesired consequence other than the one for which a compound or treatment is intended. In certain embodiments, the invention is directed to towards the treatment of pain with an MOPr agonist with reduced adverse side effect(s) that are associated with opioid treatment. In particular, a MOPr agonist having reduced adverse side effect(s) is a peptide of the invention. In certain embodiments, the invention provides a method of treating pain or delivering analgesia with reduced adverse side effect(s), preferably reduced in comparison to morphine, comprising administering to a subject a peptide of the invention or a pharmaceutical composition comprising a peptide of the invention. In certain embodiments, the invention provides a method of treating pain or delivering analgesia with reduced adverse side effect(s), preferably reduced in comparison to morphine, comprising administering a therapeutically effective amount of a peptide of the invention or a pharmaceutical composition comprising a peptide of the invention to a subject in need thereof. In certain embodiments, the invention provides use of a peptide of the invention or a pharmaceutical composition comprising a peptide of the invention for the manufacture of a medicament for a method of treating pain or delivering analgesia with reduced adverse side effect(s), preferably reduced in comparison to morphine. In certain embodiments, the invention provides a peptide of the invention or a pharmaceutical composition comprising a peptide of the invention for use in a method of treating pain or delivering analgesia with reduced adverse side effect(s), preferably reduced in comparison to morphine. Adverse side effects that are associated with opioid treatment, including MOPr agonists, include tolerance, gastrointestinal (GI) inhibition and constipation, respiratory depression, motor disturbances, opioid-induced hyperalgesia, abuse potential, and/or dependence. Preferably, a peptide of the invention reduces one or more adverse side effect(s) that are associated with opioid treatment. More preferably, a peptide of the invention reduces gastrointestinal (GI) inhibition and/or respiratory depression that are associated with opioid treatment. The method by which a reduction in an adverse side effect is assessed is not particularly limited and may be determined by any method known in the art. The adverse side effect profile of morphine is well known in the art. A reduction in one or more adverse side effect(s) associated with opioid treatment may be determined by comparing the adverse side effect(s) of a peptide of the invention with morphine in a suitable assay, including, but not limited to, an in vivo animal model of pain treatment, an in vivo animal model of gastrointestinal (GI) inhibition and an in vivo animal model of respiratory depression.

The peptides of the invention may be formulated for any suitable method of administration. The peptides of the present invention may be administered orally, parenterally, topically, rectally, nasally, buccally, vaginally, transdermally, transmucosally, or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In certain embodiments, the peptides of the invention are preferably formulated as for injection. When the peptides of the invention are formulated for injection, the formulations may be administered subcutaneously, intraperitoneally, intravenously, or intrathecally. In certain embodiments, the peptides of the formulation are formulated for oral administration.

Glycosylated peptides of the invention are preferably formulated for administration orally, by injection, or intrathecally. Non-glycosylated peptides of the invention are preferably formulated for administration nasally or intrathecally.

### Definitions

Unless the context clearly requires otherwise, throughout the description and the claims, the words “comprise”, “comprising” and the like are to be construed in an inclusive sense as opposed to an exclusive or exhaustive sense; that is to say, in the sense of “including, but not limited to”.

As used herein, the term “about” can mean within 1 or more standard deviation per the practice in the art. Alternatively, “about” can mean a range of up to 20%. For example, a peptide comprising additionally 1 to about 35 amino acid residues, includes a peptide comprising from 1 to about 33, 34, 35, or 36, additional amino acid residues.

As used herein, the term “L-amino acid” refers to the L isomer of an amino acid. The skilled person will understand that this refers to the stereochemistry of the alpha-carbon of the amino acid. The person skilled in the art will be familiar with well-known abbreviations, including L-alanine (L-Ala), L-valine (L-Val), L-leucine (L-Leu), L-isoleucine (L-Ile), L-Serine (L-Ser), L-Threonine (L-Thr), L-phenylalanine (L-Phe), L-tyrosine (L-Tyr), L-asparagine (L-Asn), L-glutamine (L-Gln), L-histidine (L-His), L-lysine (L-Lys), L-arginine (L-Arg), L-proline (L-Pro), L-Cysteine (L-Cys), L-methionine (L-Met), L-tryptophan (L-Trp), L-aspartic acid (L-Asp), L-glutamic acid (L-Glu), L-selenocysteine (L-Sec), L-hydroxylysine (L-Hyl) and L-hydroxyproline (L-Hyp). As used herein, the term “L-amino acid residue” refers to an L-amino acid incorporated into a peptide. As used herein the term “L-Dmt” refers to 2,6-dimethyl-L-tyrosine.

As used herein, the term “D-amino acid” refers to the D isomer of an amino acid. The skilled person will understand that this refers to the stereochemistry of the alpha-carbon of the amino acid. The person skilled in the art will be familiar with well-known abbreviations, including D-alanine (D-Ala), D-valine (D-Val), D-leucine (D-Leu), D-isoleucine (D-Ile), D-Serine (D-Ser), D-Threonine (L-Thr), D-phenylalanine (D-Phe), D-tyrosine (D-Tyr), D-asparagine (D-Asn), D-glutamine (D-Gln), D-histidine (D-His), D-lysine (D-Lys), D-arginine (D-Arg), D-proline (D-Pro), D-Cysteine (D-Cys), D-methionine (D-Met), D-tryptophan (D-Trp), D-aspartic acid (D-Asp), D-glutamic acid (D-Glu), D-selenocysteine (D-Sec), D-hydroxylysine (D-Hyl) and D-hydroxyproline (D-Hyp). As used herein, the term “D-amino acid residue” refers to a D-amino acid incorporated into a peptide.

As used herein, the term “amino acid residue” refers to an amino acid comprised in a peptide. The amino and/or carboxyl group of an amino acid residue will be part of a peptide bond. The skilled person will understand that the N- or C-terminus of a peptide can be extended by the addition of further amino acid residues by the formation of a peptide bond between the peptide and a further amino acid.

In referring to a peptide of the invention, amino acids or amino acid residues comprised in the peptides are referred to by the well-known single letter amino acid code or the three letter amino acid code with “L-” or “D-” designation. The skilled person understands that as the alpha carbon of the amino acid glycine is not asymmetric, glycine is designated as Gly in the three letter code. For example, “L-Phe-D-Val-L-Val-D-Phe” refers to a tetrapeptide consisting of L-phenylalanine-D-valine-L-valine-D-phenylalanine. Alternatively, in referring to a peptide of the invention using the well-known single letter amino acid codes, the D or L stereochemistry is distinguished by using capital letters to designate L-amino acid residues and lower-case letters to designate D-amino acid residues. For example, FvVf refers to tetrapeptide consisting of L-phenylalanine-D-valine-L-valine-D-phenylalanine. As the amino acid glycine does not have an asymmetric alpha carbon atom in the single letter code it is designated with a capital G. To distinguish between a peptide in which the C-terminus has not been modified, a peptide with a C-terminal carboxyl group (—C(═O)OH moiety), and a peptide in which the C-terminus is modified by amidation, a peptide with a C-terminal amido group (—C(═O)NH₂ moiety), in both the single amino acid code and the three letter code the peptide is shown with a terminal —NH₂, for example as FvVf-NH₂ and L-Phe-D-Val-L-Val-D-Phe-NH₂, to indicate that the C-terminus is amidated. In contrast, when the C-terminus is not modified, the peptide can be depicted with or without a terminal —OH, as for example FvVf-OH or FvVf or L-Phe-D-Val-L-Val-D-Phe or L-Phe-D-Val-L-Val-D-Phe-OH.

As used herein, the term “side chain of an amino acid” refers to the part of an amino acid or amino acid residue starting with the beta atom. The skilled person is familiar with the structure of an amino acid, which can be depicted as H₂N—C_{α}H(Rₐₐ)—C(═O)OH wherein Rₐₐ is the side chain of the amino acid. When the term “side chain of an amino acid” is used with reference to an amino acid residue having “D” or “L” stereochemistry, the skilled person understands that the side chain of the amino acid glycine is excluded, which is when Rₐₐ is hydrogen, is excluded as the amino acid glycine is not asymmetric. In the peptides of the invention the side chain of an amino acid includes the side chains of naturally occurring and non-naturally occurring amino acids. Preferably, the side chain is the side chain of a hydrophobic amino acid, including but not limited to the side chain of alanine, valine, norvaline, leucine, norleucine, and isoleucine.

As used herein, the term “monosaccharide” refers to a basic carbohydrate unit. As used herein, the term “monosaccharide moiety” refers to the monosaccharide linked to a peptide of the invention. Suitable monosaccharides for glycosylation of the peptides of the invention include, but are not limited to, dihydroxyacetone, glyceraldehydes, aldotriose, erythrulose, erythrose, threose, ribulose, psicose, xylose, glucose (Glc), fructose, mannose, galactose, fucose, ribose, tagatose, arabinose, rhamnose, sedoheptalose and nonoses such as neuraminic acid, sialic acid. A preferred monosaccharide is glucose.

As used herein, the term “disaccharide” refers to a carbohydrate formed when two monosaccharides are joined by a glycosidic linkage. As used herein, the term “disaccharide moiety” refers to the disaccharide linked to a peptide of the invention. Suitable disaccharides for glycosylation of the peptides of the invention include, but are not limited to sucrose, trehalose, saccharose, maltose, lactose (Lac), cellobiose, gentibiose, isomaltose, melibiose, and primeveose. Preferred disaccharides for glycosylation of the peptides of the invention include lactose and melibiose. In certain embodiments the disaccharide is lactose.

As used herein, the term “trisaccharide” refers to a carbohydrate formed when three monosaccharides are joined by two glycosidic linkages. As used herein, the term “trisaccharide moiety” refers to the trisaccharide linked to a peptide of the invention. Suitable trisaccharides for glycosylation of the peptides of the invention include, but are not limited to, maltotriose and raffinose.

As used herein, the term “sugar moiety” refers to a monosaccharide, disaccharide, or trisaccharide linked to a peptide of the invention. Preferably, the sugar moiety is linked to a peptide of the invention by O-linked glycosylation and by beta linkage. As used herein, a peptide of the invention comprising a sugar moiety may alternatively be referred to as a peptide comprising glycosylation or a glycosylated peptide.

As used herein, the term “bio-reversible moiety” refers to a moiety that is attached to a peptide of the invention, which upon in vivo administration, is metabolized or otherwise converted, eg by hydrolysis in blood, by metabolism in cells, or in cerebrospinal fluid, or by a combination these routes, to the biologically, pharmaceutically or therapeutically active form of the peptide of the invention. A bio-reversible moiety suitable for a peptide of the invention, includes but is not limited to, a carbonate, carbamate, imine, ether, ester, and amide moiety. In the peptides of the invention, bio-reversible moieties substituted on the N-terminus of the peptide include, but are not limited to,

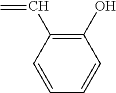

(imine moiety) or ═N═N (azido moiety), —C(═O)OZ_{(1, 3, or 5)} or —C(═O)OCH₂OC(═O)Z_{(2, 4, or 6)}. It is understood that the N-terminal Nitrogen of the peptide is bound to the groups. Z_{(1, 3, or 5)} is C₁-C₆ alkyl or aryl, preferably —CH₂CH₃. Z_{(2, 4, or 6)} is C₁-C₆ alkyl or aryl, preferably —CH₃. In the peptides of the invention, bio-reversible moieties may be substituted on the hydroxy of L-tyrosine or 2,6-dimethyl-L-tyrosine and include, but are not limited to, —C(═O)Z₍₇₎. Z₍₇₎ is C₁-C₆ alkyl or aryl, preferably —CH₃.

As used herein, the term “alkyl” refers to the radical of saturated aliphatic groups, including straight-chain alkyl groups, branched-chain alkyl groups, and cycloalkyl (alicyclic) groups. In certain embodiments, the alkyl moiety is optionally glycosylated by any method known in the art. It is understood that in embodiments in which the alkyl moiety is glycosylated, the alkyl moiety has a substituent replacing a hydrogen on one or more carbons of the hydrocarbon backbone to allow for glycosylation. As used herein the term “alkylation” or “alkylated” and the like in the context of N-terminus of a peptide and/or an amino acid residue, refers to replacing one or both N-terminal hydrogens with an alkyl group and in the context of a —OH group refers to replace the hydrogen with an alkyl group such as methyl (“methylated”; “methylation”), ethyl (“ethylated”, “ethylation”), and the like. Singly alkylated or single alkylation and the like refers to replacing one of the N-terminal hydrogens of a peptide or amino acid residue with an alkyl group. Singly methylated refers to replacing one of the N-terminal hydrogens with —CH₃.

As used herein, the term “aryl” includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. In certain embodiments, the aryl is optionally glycosylated by any method known in the art.

As used here, the term “linker” refers to any suitable linker known in the art. Suitable linkers include amino acid based linkers, including but not limited to single amino acid linkers, such as L-Cysteine, L-lysine, L-Serine, L-threonine, and the like, peptide based linkers including but not limited to L-Valine-L-Citrulline, L-Phe-L-Lys, L-Glutamic acid-L-Valine-L-Citrulline, and the like, amino acid comprising linkers, including but not limited to valine-citrulline-p-aminocarbamate (VC-PABC), and the like, and maleimide based linkers, including but not limited to maleimidocaproyl, maleimidomethyl cyclohexane-1-carboxylate and the like; as well as combinations of such linkers such as maleimidocaproyl-valine-citrulline-p-aminocarbamate, as well as amino and carboxy group containing linkers such as 6-aminohexanoic acid, and the like. The skilled person will appreciate that maleimide based linkers may use a L-cysteine residue such that maleimide is bonded to the sulphur of the L-cysteine or may use a L-Lysine residue such that the maleimide is bonded to the nitrogen of the L-lysine. In embodiments comprising a maleimide based linker, the peptide may further comprise a C-terminal L-cysteine residue or L-lysine residue that is bonded to the maleimide based linker, such as maleimidocaproyl, maleimidomethyl cyclohexane-1-carboxylate. In peptides disclosed herein the linker further comprises a sugar moiety.

As used herein, the term “selective MOPr agonist” refers to an agonist that is selective for the MOPr over at least one of the related κ-opioid (KOPr) and/or δ-opioid (DOPr) subtypes. Selectivity of an MOPr agonist can be determined by methods well known in the art. In one exemplary method, the Kᵢ of a peptide for MOPr, preferably hMOPr can be determined in a competitive binding assay with [²H]DAMGO and compared to the Kᵢ of the same peptide for (2) DOPr, preferably human DOPr (hDOPr), which can be determined in a competitive binding assay with [³H]DADLE, and/or (3) KOPr, preferably human KOPr (hKOPr), which can be determined in a competitive binding assay with [³H]U69593. Peptides of the invention that are MOPr agonists may also be selective MOPr agonists. Preferably, a peptide of the invention is a MOPr agonist that is selective for MOPr over at least one of κ-opioid (KOPr) or δ-opioid (DOPr). More preferably, a peptide of the invention is a MOPr agonist that is selective for MOPr over both κ-opioid (KOPr) and δ-opioid (DOPr). Most preferably, a peptide of the invention exhibits 50-fold selectivity for MOPr over KOPr and/or 50-fold selectivity for MOPr over DOPr.

As used herein, the term a G protein-biased MOPr agonist refer to a peptide that differentially agonizes the G protein-coupled receptor (GPCR) to couple to distinct downstream signaling pathways. G protein-biased MOPr agonist peptides of the exhibit increased signaling via G-proteins versus β-arrestin recruitment. Assays to assess biased G-protein peptides are known in the art and include, but are not limited to, comparisons of G-protein activation assay, MOPr C-terminal phosphorylation, β-arrestin recruitment, and/or MOPr internalisation. The G-protein bias of a peptide can be compared in a MOPr C-terminal phosphorylation assay to morphine, which is known to weakly phosphorylate MOPr at Ser375, endomorphin-2, which is known to strongly phosphorylate MOPr at Ser375, and/or met-enkephalin, which is known to strongly phosphorylate MOPr at Ser375. The G-protein bias of a peptide can be compared in a β-arrestin recruitment assay to morphine, which is known to weakly induce β-arrestin recruitment, endomorphin-2, which is known to strongly induce β-arrestin recruitment, and/or met-enkephalin, which is known to strongly induce β-arrestin recruitment. The G-protein bias of a peptide can be compared in a MOPr internalisation assay to morphine, which is known to weakly induce MOPr internalisation, endomorphin-2, which is known to strongly induce MOPr internalisation, and/or met-enkephalin, which is known to strongly induce MOPr internalisation. The G-protein bias of a peptide can be compared in a G-protein activation assay to morphine, endomorphin-2, and/or met-enkephalin, each of which is known to activate G-proteins. Preferably, a peptide of the invention, in comparison to morphine, exhibits a lower ratio of induction of C-terminal phosphorylation of MOPr versus G-protein activation; and/or exhibits a lower ratio of induction of β-arrestin recruitment versus G-protein activation; and/or exhibits a lower ratio of induction of MOPr internalisation versus G-protein activation.

As used herein, the term “pharmaceutically acceptable excipient” encompasses any carrier, excipient, diluent, filler, salt, buffer, stabilizer, solubilizer, lipid, stabilizer, or other material well known in the art for use in pharmaceutical compositions. The choice of an excipient for use in a composition will depend upon the intended route of administration for the composition. The preparation of pharmaceutically acceptable excipients and compositions containing these materials is described in, e.g., Remington's Pharmaceutical Sciences, 22^{nd} Edition, ed. University of the Sciences in Philadelphia, Lippincott, Williams & Wilkins, Philadelphia Pa., 2012.

As used herein, the term “subject” or “subject in need thereof” refers to a mammal, including, for example, farm animals, such as sheep, pigs, cows, and horses; pet animals, such as dogs and cats; laboratory animals, such as rats, mice and rabbits, and humans. Preferably, the subject is a dog, cat, or human. More preferably, the subject is human.

As used herein, the term “therapeutically effective amount” means the amount of a peptide of the invention or pharmaceutical composition comprising a peptide of the invention that will elicit the biological or medical response of a subject in need thereof that is being sought by the researcher, veterinarian, medical doctor or other clinician.

### BRIEF DESCRIPTION OF THE FIGURES

Embodiments of the invention will now be described, by way of example only, with reference to the accompanying drawings as follows.

FIG. 1: Competitive Binding Assay. Peptides [YvVf-OH (3a), YvVf-NH₂ (3b), [Dmt]-vVf-NH₂ (3c)] tested for competitive binding to hMOPr against the MOPr agonist [³H]DMAGO. [Dmt]-vVf-NH₂ (3c) was tested for competitive binding to hDOPr against the DOPr agonist [³H]DADLE and for competitive binding to hKOPr against the KOPr agonist [³H]U69593. Symbols in figure: hMOPr: YvVf-OH (3a) x in circle; YvVf-NH₂ (3b) circle in circle, [Dmt]-vVf-NH₂ (3c) large circle; DOPr: [Dmt]-vVf-NH₂ (3c) * in circle; KOPr: [Dmt]-vVf-NH₂ (3c)+in circle.

FIG. 2: FIG. 2A: Example of GIRK current recorded from rat LC neuron in response to met-enkephalin (1 μM), [Dmt]-vVf-NH₂ (3c, Bilorphin) (1 μM), and its reversal by co-application of the MOPr selective antagonist, CTAP ((D-Phe-Cys-Tyr-D-Trp-Arg-Thr-Pen-Thr-NH₂) (SEQ ID NO: 31) (1 μM). Scale bars: 50 pA, 5 min. FIG. 2B: Partial antagonist effect of [Dmt]-vVf-NH₂ (3c, Bilorphin) (1 μM) on the GIRK current evoked by supramaximal desensitizing concentration of met-enkephalin (10 μM, 10 min; same scale as FIG. 2A).

FIG. 3: Agonist concentration-response relationships of exemplar opioids and [Dmt]-vVf-NH₂ (3c, Bilorphin) for activation of GIRK current in LC neurons normalised to 1 μM met-enkephalin applied as a probe in each cell (N=4-13 cells per data point). [Dmt]-vVf-NH₂ (3c, Bilorphin, * in circle) in comparison to met-enkephalin (X in circle), morphine (+ in circle), and endomorphin-2 (circle in circle).

FIG. 4: Exemplary record of G_{GIRK} in mMOPr expressing AtT20 cell in response to somatostatin (SST) and the concentrations of opioids shown and duration of bars, after alkylation of a fraction of receptors by the irreversible MOPr antagonist β-chlornaltrexamine (β-CNA). Scale bar 0.2 ns, 1 min.

FIG. 5: Concentration-response curves of G_{GIRK} induced by opioids in AtT20 cells after reducing the receptor reserve by β-CNA pretreatment to produce a maximum response to met-enkephalin to 80% of that produced by somatostatin (SST). [Dmt]-vVf-NH₂ (3c, Bilorphin, * in circle (purple)) in comparison to met-enkephalin (X in circle), morphine (+ in circle), endomorphin-2 (circle in circle) and oliceridine (large circle). Patch-clamp recordings in AtT20 cells stably expressing FLAG tagged mouse MOPr (mMOPr).

FIG. 6: C-terminal phosphorylation induction by [Dmt]-vVf-NH₂ (3c, Bilorphin) in comparison to met-enkephalin, morphine, and endomorphin-2 using a phosphosite specific antibody. Representative images of Serine 375 phosphorylation in AtT20 cells induced by a saturating concentration (30 μM) of met-enkephalin, endomorphin 2, morphine and bilorphin after 5 min incubation. Colours enhanced uniformly for presentation purposes.

FIG. 7: β-Arrestin recruitment induced by [Dmt]-vVf-NH₂ (3c, Bilorphin) in comparison to met-enkephalin, morphine, and endomorphin-2 as determined by MOPr-luciferease and β-arrestin2-YFP constructs. Time course of ligand-induced BRET signal (light emission of 535 nm/475 nm) indicating β-arrestin 2 recruitment after agonist exposure (shown by the arrow). The band represents the standard error of experiments repeated independently 6 times (each experiment had triplicates).

FIG. 8: MOPr internalisation. Example images of MOPr internalisation 30 min after treatment with 30 μM of agonists. Dual staining was employed for quantification (membrane receptor in green (appearing light grey) and internalized receptor in red (appearing darker grey), colours enhanced uniformly for presentation purposes).

FIG. 9: Maximal efficacy values of endomorphin 2, morphine and bilorphin relative to met-enkephalin for GIRK channel activation, Serine 375 phosphorylation, β-arrestin 2 recruitment and normalization internalisation. [Dmt]-vVf-NH₂ (3c, Bilorphin) in comparison to morphine, and endomorphin-2, and met-enkephalin, presented in order from foreground to background).

FIG. 10: MOPr internalisation in cells expressing GRK2-YFP. Examples of enhanced internalization (green (appearing light grey) and red (appearing darker grey) as in panel C) produced by oliceridine, bilorphin and morphine in cells overexpressing both GRK2 (yellow (appearing grey)) and β-arrestin 2.

FIG. 11: FIG. 11A: Internalization for each agonist in cells (ratio of fluorescence in green/[green+red] channels) transiently transfected with both GRK2 and β-arrestin 2 (n=40 cells from 2 experiments). FIG. 11B: Bias ratios calculated from GGIRK maxima normalized to met-enkephalin (from FIG. 5) internalization (from FIG. 11A) normalized to Met-enkephalin for bilorphin indicates greater G-protein bias than both olicerideine and morphine. FIG. 11C: Internalization for each agonist in cells (ratio of fluorescence in green/[green+red] channels) transiently transfected with both GRK2 and β-arrestin 2 (n=5 experiments, with greater than 10 cells in each). FIG. 11D: Bias ratios calculated from GGIRK maxima normalized to met-enkephalin (from FIG. 5) and internalization (from FIG. 11C) normalized to Met-enkephalin for bilorphin indicates greater G-protein bias than both olicerideine and morphine.

FIG. 12: In vivo analgesia assay of analogues of [Dmt]-vVf-NH₂ (3c, Bilorphin). [Dmt]-vVf-L-Ser(β-Lac)-NH₂ (3g, Bilactorphin) produced dose-dependent analgesia in mice on the 54° C. hotplate, after sub-cutaneous administration and was antagonised by naltrexone. Doses in μmol/kg are indicated in parentheses (n=7-12 per data point except naltrexone [n=4]). Vehicle (circle with =), morphine (circle with +), [Dmt]-vVf-L-Ser(β-Lac)-NH₂ (3g, Bilactorphin (14 μmol/kg, circle with 1 star (*); 28 μmol/kg, circle with 2 stars (**); 56 μmol/kg, circle with 3 stars (***); 112 μmol/kg, circle with 4 stars (****)); [Dmt]-vVf-L-Ser(β-Lac)-NH₂ (3g, Bilactorphin) with naltrexone (circle with x).

FIG. 13: Peripherally administered [Dmt]-vVf-L-Ser(β-Lac)-NH₂ (3g, Bilactorphin) was equipotent with morphine on the hotplate test (n=5-12). [Dmt]-vVf-L-Ser(β-Lac)-NH₂ (3g, Bilactorphin, large circle (dark grey)) and morphine (x in circle (light grey)).

FIG. 14: Representative trace indicating time course of GIRK current in MOPr expressing AtT20 cell in response to [Dmt]-vVf-NH₂ (3c, Bilorphin) and [Dmt]-vVf-L-Ser(β-Lac)-NH₂ (3g, Bilactorphin) and morphine relative to a probe of 1 μM somatostatin. The scale bars represent 0.2 nS and 1 min.

FIG. 15: Concentration response curves of potassium conductance induced by [Dmt]-vVf-NH₂ (3c, Bilorphin, x in circle) and [Dmt]-vVf-L-Ser(β-Lac)-NH₂ (3g, Bilactorphin, large circle) normalised to 1 μM somatostatin applied as a probe in individual cells.

FIG. 16: Example images of MOPr internalization induced by 30 μM [Dmt]-vVf-NH₂ (3c, Bilorphin) and [Dmt]-vVf-L-Ser(β-Lac)-NH₂ (3g, Bilactorphin) after GRK2 overexpression (membrane and normalised MOPr in green (appearing light grey) and red (appearing darker grey) respectively and GRK2 in Yellow).

FIG. 17: Maximal efficacy values of morphine (green), [Dmt]-vVf-NH₂ (3c, Bilorphin, purple) and [Dmt]-vVf-L-Ser(β-Lac)-NH₂ (3g, Bilactorphin, dark green) relative to met-enkephalin (30 μM exposure of the agonists) to produce receptor internalisation. For each set, morphine on the left, bilprophin is in the middle, and bilactorphin is on the right.

FIG. 18: Predicted binding pose of [Dmt]-vVf-NH₂ (3c, Bilorphin) (A and B) and endomorphin-2 (C and D) from MD simulations. FIGS. 18A and 18C: Predicted binding poses of bilorphin (dark grey) (18A) and endomorphin-2 (light grey) (18C), and the positions of the surrounding binding pocket residues (lightest grey) obtained after molecular docking and 1 μs of MD simulations. The salt bridge between protonated amine of the ligands and Asp147^{3.32} is marked as a dashed black line. TM7 has been removed for clarity. FIGS. 18B and 18D: Alternative viewpoint from (18A/18C) of the predicted binding poses of bilorphin (dark grey) (18B), and endomorphin-2 (light grey) (18D), and the positions of the surrounding binding pocket residues (lightest grey) obtained after molecular docking and 1 μs of MD simulations. The salt bridge between protonated amine of the ligands and Asp147^{3.32} is marked as a dashed black line. This time TM4 has been removed for clarity.

FIG. 19: RMSD plot of [Dmt]-vVf-NH₂ (3c, Bilorphin) (A) and endomorphin-2 (B). FIG. 19A: RMSD calculations performed on the heavy atoms of bilorphin, compared to the initial docked pose (darker grey), and the alpha carbons of the receptor transmembrane domains, compared to the first frame of the MD simulation (lighter grey). FIG. 19B: RMSD calculations performed on the heavy atoms of endomorphin-2, compared to the initial docked pose (lighter grey), and the alpha carbons of the receptor transmembrane domains, compared to the first frame of the MD simulation (grey). Inset: fluctuations of Phe⁴ in endomorphin-2 during the MD simulation showing 3 different positions of Phe⁴.

FIG. 20: FIG. 20A: Ligand-residue interaction fingerprints for the bilorphin-MOPr complex (dark grey) and endomorphin-2-MOPr complex (light grey). Data is expressed as the percentage of simulation time each residue is within 4.5 Å of the ligand, with points radiating outwards from 0% to 100% in 20% increments. FIG. 20B: Principal component analysis was performed on the alpha carbons of the receptor transmembrane domains, before projecting the receptor conformations at each simulation time point onto PC1 and PC2. The bilorphin-MOPr complex is in purple, the endomorphin-2-MOPr complex in orange, and the black point indicates the conformation of the inactive MOPr model to which the peptides were docked.

FIG. 21: Extracted structures representing the extremes of PC1 demonstrate the conformational differences between the bilorphin-MOPr complex (dark grey) and the endomorphin-2-MOPr complex (light grey). Loops have been removed from the image to depict only the part of the receptor the PCA was performed on. White arrows indicate conformational changes in the helices moving from bilorphin—bound to endomorphin-2-bound MOPr.

FIG. 22: Calculation of the volume of the orthosteric binding site using CASTp showed the binding pocket was larger for the bilorphin-MOPr complex (dark grey) compared to the endomorphin2-MOPr complex (light grey). CASTp calculations were performed on structures averaged over the final 100 ns of each simulation.

FIG. 23: Maximal effect of agonists in each signalling and calculation of bias for G-protein activation versus other pathways: Non-normalized maximal efficacy (±S.E.M.) for activation of A, GIRK, B, Ser³⁷¹ phosphorylation, C β-arrestin 2 recruitment and D internalization that was used to calculate ratios presented in FIG. 9, and for calculation of Δ Normalized E_{Max} in E, or included in the operational model in F. Data represented in E and F are mean and 95% confidence intervals. Met-enkephalin is shown in lightest grey, endomorphin 2 in dark grey, morphine in lighter grey and bilorphin (peptide 3c, [Dmt]-vVf-NH₂) is shown in darkest grey.

FIG. 24: Antinociceptive action of oral bilactorphin and morphine: FIG. 24A: Time-response (mean±SEM) for oral gavage of [Dmt]-vVf-L-Ser(β-Lac)-NH₂ (3g, Bilactorphin) and morphine on hot-plate latency. Vehicle (large circle); Morphine (circle with +); [Dmt]-vVf-L-Ser(p-Lac)-NH₂ (3g, Bilactorphin) (100 μmol/kg, 6, circle with 1 star (*); 300 μmol/kg, 6, circle with 2 stars (**); 1000 μmol/kg, 6, circle with 3 stars (***)). FIG. 24B: Area under the curve (AUC) of the full time-response data for each animal shown in FIG. 24A for 300 min after gavage. Ordinary one-way ANOVA of AUC data revealed statistically significant differences between all doses of bilactorphin above 100 μmol/kg and morphine 90 μmol/kg.

FIG. 25: Structures of bilaids, bilorphin, and bilactorphin, including
L-Phe-D-Val-L-Val-D-Phe (peptide 1a, Bilaid A);
L-Phe-D-Val-L-Val-D-Phe-NH₂ (peptide 1e);
L-Tyr-D-Val-L-Val-D-Phe (peptide 3a, Bilaid C);
L-Tyr-D-Val-L-Val-D-Phe-NH₂ (peptide 3b);
2,6-dimethyl-L-tyrosine-D-Val-L-Val-D-Phe-NH₂ (peptide 3c; Bilorphin); and
2,6-dimethyl-L-tyrosine-D-Val-L-Val-D-Phe-L-Ser(β-Lac)-NH₂ (peptide 3g; Bilactorphin).

FIG. 26: Analogues of Bilaid C. Including the following peptides:
2,6-dimethyl-L-tyrosine-D-Val-L-Val-D-Phe-OCH₂CH₃ (peptide 4);
2,6-dimethyl-L-tyrosine-D-Val-L-Val-D-Phe-NH₂ wherein the hydroxy group on 2,6-dimethyl-L-tyrosine is substituted with the bio-reversible moiety —C(═O)CH₃ (peptide 5);
2,6-dimethyl-L-tyrosine-D-Val-L-Val-D-Phe-OCH₂CH₃ wherein the hydroxy group on 2,6-dimethyl-L-tyrosine is substituted with the bio-reversible moiety —C(═O)CH₃ (peptide 6);
2,6-dimethyl-L-tyrosine-D-Val-L-Val-D-Phe-NH₂ wherein the N-terminus is substituted with the bio-reversible moiety —C(═O)OCH₂CH₃ (peptide 7);
2,6-dimethyl-L-tyrosine-D-Val-L-Val-D-Phe-NH₂ wherein the N-terminus is substituted with the bio-reversible moiety

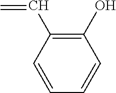

(peptide 8);
2,6-dimethyl-L-tyrosine-D-Val-L-Val-D-Phe-NH₂ wherein the N-terminus is substituted with the bio-reversible moiety, ═N═N, to form an N-terminal azido group (peptide 9);
2,6-dimethyl-L-tyrosine-D-Val-L-Val-D-Phe-NH₂ wherein the N-terminus is substituted with the bio-reversible moiety —C(═O)OCH₂OC(═O)CH₃ (peptide 10);
2,6-dimethyl-L-tyrosine-D-Val-L-Val-D-Phe-L-Pro-L-Asn-L-Leu-L-Ala-L-Glu-L-Lys-L-Ala-L-Leu-L-Lys-L-Ser-L-Leu-NH₂ (peptide 11);
2,6-dimethyl-L-tyrosine-D-Val-L-Val-D-Phe-L-Ser(β-Lac)-NH₂ (peptide 3g; Bilactorphin); and
2,6-dimethyl-L-tyrosine-D-Val-L-Val-D-Phe-L-Ser(β-D-Glc)-NH₂ (peptide 3h).

FIG. 27: Antinociceptive response of the peptides as labelled presented as integrated Area Under the Curve over one hour (AUC response in seconds×time in minutes) for hotplate responses measured, 5, 10, 20, 30 and 60 minutes after subcutaneous injection of each peptide or saline. Asterisks show significantly different AUC response from saline (One way ANOVA with Fisher's LSD post-hoc tests).

FIG. 28: Cryo-EM structure and Molecular Dynamics simulations with DAMGO: A. The binding pose of DAMGO in the cryo-EM structure of the MOPr-Gi complex (Koehl Nature (2018) 558: 547-552). DAMGO is shown in dark grey, with surrounding binding pocket residues and the receptor helices in light grey. B. Predicted binding pose of DAMGO after docking with BUDE and 1 μs MD simulation starting from the inactive MOPr structure (Manglik Nature (2012) 485: 321-326.) DAMGO is shown in middle grey and surrounding residues and helices in lighter grey. Koehl Nature (2018) 558: 547-552 reported poor resolution of the C-terminal portion of DAMGO and high flexibility of this region in an MD simulation. With this flexible C-terminal ethanolamine omitted, the RMSD between all heavy atoms of DAMGO in the cryo-EM structure and in our final pose after 1 μs MD was 2.83 Å. Thus the DAMGO-MOPr interactions in the cryo-EM structure and in the model were virtually identical.

### DETAILED DESCRIPTION OF PREFERRED EMBODIMENTS

The following are embodiments of the invention.

Embodiment 1: An isolated peptide comprising Formula I

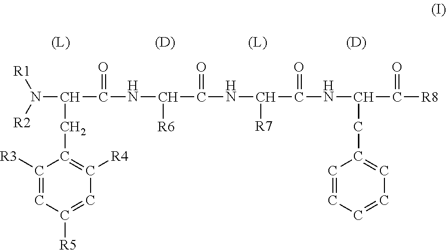

wherein, counting from the N-terminus, the first and third amino acid residues are L-amino acid residues and the second and fourth amino acid residues are D-amino acid residues; wherein
R¹ is hydrogen or a bio-reversible moiety optionally comprising a sugar moiety;
R² is hydrogen or a bio-reversible moiety optionally comprising a sugar moiety;
wherein R¹ and R² may together form one bio-reversible moiety;
   R³ and R⁴ are independently selected from hydrogen or C₁-C₃ alkyl, preferably —CH₃;
   R⁵ is hydrogen, —OH, or a bio-reversible moiety optionally comprising a sugar moiety;
   R⁶ is a side chain of an amino acid or C₁-C₆ alkyl;
   R⁷ is a side chain of an amino acid or C₁-C₆ alkyl;
   R⁸ is —OH, —NH₂, —O(C₁-C₃ alkyl),

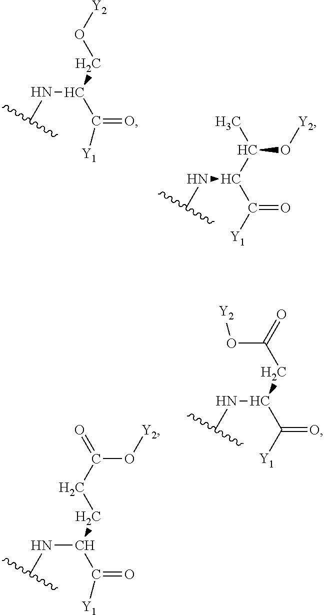

or 1 to about 30 L-amino acid residues;
Y₁ is —OH, —NH₂, or 1 to about 30 L-amino acid residues;
Y₂ is hydrogen or a sugar moiety, preferably a disaccharide moiety; and
wherein when R⁸ is 1 to about 30 L-amino acid residues (1) the L-amino acid residues are optionally residues that may be optionally glycosylated with a sugar moiety, preferably a disaccharide moiety, and (2) the C-terminus is optionally amidated.

Embodiment 2: The peptide according to Embodiment 1, wherein R⁶ is C₁-C₆ alkyl and R⁷ is C₁-C₆ alkyl.

Embodiment 3: The peptide according to Embodiment 1 or Embodiment 2, wherein R⁶ and R⁷ are independently selected from the side chain of alanine, valine, norvaline, leucine, norleucine, or isoleucine.

Embodiment 4: The peptide according to any one of Embodiments 1 to 3, wherein R⁶ and R⁷ are each a valine side chain (—CH(CH₃)₂).

Embodiment 5: The peptide according to Embodiment 1, wherein R⁶ and R⁷ are each a threonine side chain.

Embodiment 6: The peptide according to any one of Embodiments 1 to 5, wherein R³ and R⁴ are —CH₃; and R⁵ is —OH.

Embodiment 7: The peptide according to any one of Embodiments 1 to 6, wherein R¹ and R² are each hydrogen.

Embodiment 8: The peptide according to any one of Embodiments 1 to 5, wherein R¹, R², R³, R⁴, and R⁵ are each hydrogen.

Embodiment 9: An isolated peptide comprising Formula I

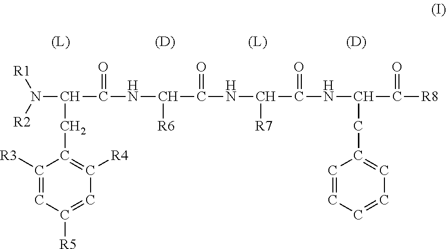

wherein, counting from the N-terminus, the first and third amino acid residues are L-amino acid residues and the second and fourth amino acid residues are D-amino acid residues; wherein
R¹ is hydrogen, single bond, or a —C₁-C₃ alkyl;
R² is hydrogen, single bond, or a —C₁-C₃ alkyl;
R³ and R⁴ are independently selected from hydrogen or C₁-C₃ alkyl, preferably —CH₃;
R⁵ is hydrogen, —OH, or —O(C₁-C₃)alkyl;
R⁶ is a side chain of an amino acid or C₁-C₆ alkyl;
R⁷ is a side chain of an amino acid or C₁-C₆ alkyl;
R⁸ is —OH, —NH₂, —O(C₁-C₃ alkyl),

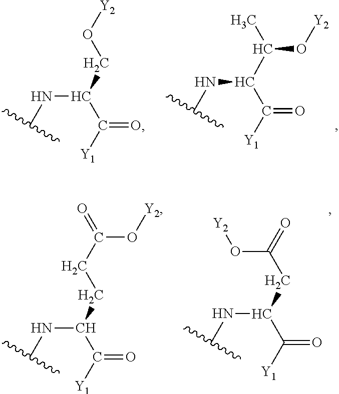

or 1 to about 30 L-amino acid residues;
Y₁ is —OH, —NH₂, or 1 to about 30 L-amino acid residues;
Y₂ is hydrogen or a sugar moiety, preferably a disaccharide moiety;
   wherein when R⁸ is 1 to about 30 L-amino acid residues (1) the L-amino acid residues are optionally residues that may be optionally glycosylated with a sugar moiety, preferably a disaccharide moiety, and (2) the C-terminus is optionally amidated;
   wherein when R⁸ is a linker, the linker comprises a sugar moiety, preferably a disaccharide moiety such as lactose, and
   wherein when one of R¹ or R² is a single bond, one of R¹ and R² is hydrogen and the single bond is a peptide bond to an L-amino acid residue that may optionally be N-terminally alkylated, preferably singly methylated.

Embodiment 10: The peptide according to Embodiment 9, wherein one of R¹ and R² is hydrogen and one of R¹ and R² is —CH₃.

Embodiment 11: The peptide according to Embodiment 9 or Embodiment 10, wherein R⁵ is —O(C₁-C₃)alkyl, preferably —OCH₃.

Embodiment 12: The peptide according to Embodiment 11, wherein R³ and R⁴ are —CH₃.

Embodiment 13: The peptide according to Embodiment 11, wherein R³ and R⁴ are hydrogen.

Embodiment 14: The peptide according to Embodiment 9 or Embodiment 10, wherein R³ and R⁴ are —CH₃ and R⁵ is —OH.

Embodiment 15: The peptide according to any one of Embodiments 9 to 14, wherein R⁶ and R⁷ are each a valine side chain (—CH(CH₃)₂).

Embodiment 16: The peptide according to any one of Embodiments 9 to 14, wherein R⁶ and R⁷ are each a threonine side chain.

Embodiment 17: The peptide according to any one of Embodiments 9 to 16, wherein one of R¹ or R² is a single bond, one of R¹ and R² is a hydrogen, and the single bond is a peptide bond to an L-amino acid residue.

Embodiment 18: The peptide according to Embodiment 17, wherein the L-amino acid residue has at least one N-terminal methylation.

Embodiment 19: The peptide according to Embodiment 17 or Embodiment 18, wherein the L-amino acid residue is an L-alanine residue.

Embodiment 20: The peptide according to any one of Embodiments 9 to 19, wherein R⁸ is a linker.

Embodiment 21: The peptide according to Embodiment 20, wherein the linker comprises an amino acid based linker, peptide based linker, an amino acid comprising linker, and/or maleimide based linker, and/or a combination thereof.

Embodiment 22: The peptide according to any one of Embodiments 1 to 19, wherein R⁸ is —OH, —NH₂, —O(C₁-C₃ alkyl),

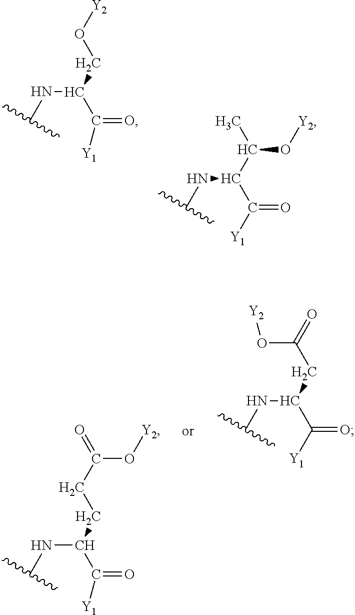

Y₁ is —OH, —NH₂, or 1 to about 30 L-amino acid residues; and
Y₂ is hydrogen or a sugar moiety, preferably a disaccharide moiety.

Embodiment 23: The peptide according to Embodiment 22, wherein R⁸ is

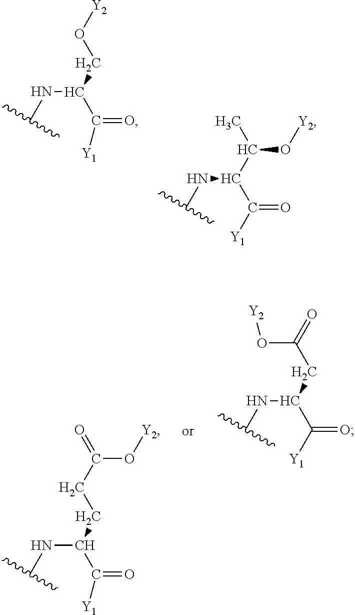

Y₁ is OH NH₂, or 1 to about 30 L-amino acid residues and
Y₂ is hydrogen or a sugar moiety, preferably a disaccharide moiety.

Embodiment 24: The peptide according to Embodiment 22 or Embodiment 23, wherein Y₁—NH₂.

Embodiment 25: The peptide according to Embodiment 22 or Embodiment 23, wherein Y₁ is 1 to about 30 L-amino acid residues.

Embodiment 26: The peptide according to Embodiment 25, wherein Y₁ is 1 to about 25 L-amino acid residues, 1 to about 20 L-amino acid residues, 1 to about 15 L-amino acid residues, 1 to about 11 L-amino acid residues, or 1 to about 5 L-amino acid residues.

Embodiment 27: The peptide according to Embodiment 26, wherein Y¹ is 1 to about 11 L-amino acid residues.

Embodiment 28: The peptide according to any one of Embodiments 1 to 19, wherein R⁸ is 1 to about 30 L-amino acid residues.

Embodiment 29: The peptide according to Embodiment 28, wherein R⁸ is 1 to about 25 L-amino acid residues, 1 to about 20 L-amino acid residues, 1 to about 15 L-amino acid residues, 1 to about 11 L-amino acid residues, or 1 to about 5 L-amino acid residues.

Embodiment 30: The peptide according to Embodiment 29, wherein R⁸ is 1 to about 11 L-amino acid residues.

Embodiment 31: The peptide according to Embodiment 30, wherein the 1 to about 11 L-amino acid residues comprise at least one glycosylated L-amino acid residue, preferably comprising at least one O-glycosylated L-serine residue.

Embodiment 32: The peptide according to Embodiment 1, wherein R¹ and R² are hydrogen; R³, R⁴, and R⁵ are hydrogen; R⁶ and R⁷ are each —CH(CH₃)₂; and R⁸ is —OH, —NH₂, —O(C₁-C₃ alkyl),

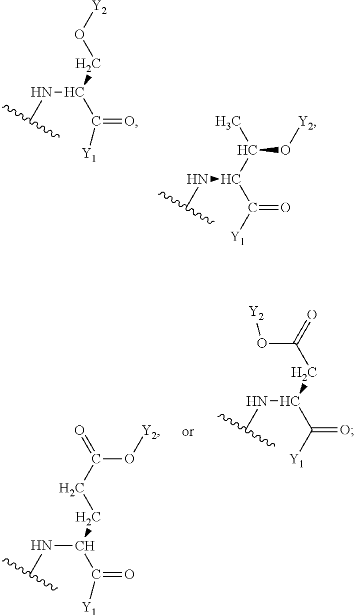

Y₁ is —OH, —NH₂, or 1 to about 30 L-amino acid residues; and
Y₂ is hydrogen or a sugar moiety, preferably a disaccharide moiety.

Embodiment 33: The peptide according to Embodiment 1, wherein R¹ and R² are hydrogen; R³ and R⁴ are both hydrogen or both —CH₃; R⁵ is —OH; R⁶ and R⁷ are each —CH(CH₃)₂; and R⁸ is —OH, —NH₂, —O(C₁-C₃ alkyl),

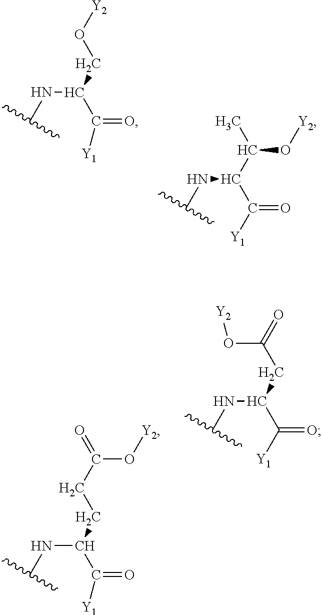

Y₁ is —OH, —NH₂, or 1 to about 30 L-amino acid residues;
Y₂ is hydrogen or a sugar moiety, preferably a disaccharide moiety.

Embodiment 34: The peptide according to Embodiment 32 or Embodiment 33, wherein R⁸ is —NH₂,

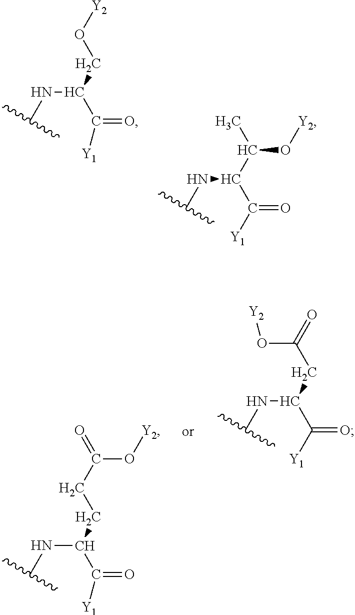

Y₁ is —OH, —NH₂, or 1 to about 30 L-amino acid residues; and
Y₂ is hydrogen or a sugar moiety, preferably a disaccharide moiety.

Embodiment 35: The peptide according to any one of Embodiments 32 to 34, wherein R⁸ is

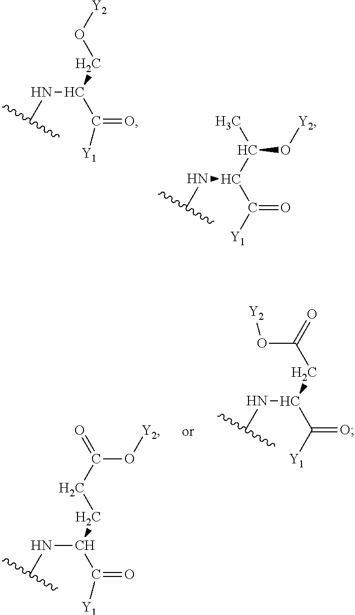

Y₁ is —OH or —NH₂; and
Y₂ is hydrogen or a sugar moiety, preferably a disaccharide moiety.

Embodiment 36: The peptide according to any one of Embodiments 32 to 34, wherein Y₁ is 1 to about 25 L-amino acid residues, 1 to about 20 L-amino acid residues, 1 to about 15 L-amino acid residues, 1 to about 11 L-amino acid residues, or 1 to about 5 L-amino acid residues.

Embodiment 37: The peptide according to Embodiment 36, wherein Y¹ is 1 to about 11 L-amino acid residues.

Embodiment 38: The peptide according to any one of Embodiments 1 to 19, 22, 23, and 32 to 35, wherein Y₂ is a sugar moiety, preferably a disaccharide moiety.

Embodiment 39: The peptide according to Embodiment 38, wherein Y₂ the disaccharide moiety is a lactose moiety or melibiose moiety.

Embodiment 40: The peptide according to Embodiment 38, wherein Y₂ the disaccharide moiety is a lactose moiety.

Embodiment 41: The peptide according to Embodiment 39 or Embodiment 40, wherein the disaccharide moiety is attached through a beta linkage.

Embodiment 42: The peptide according to Embodiment 1, wherein R⁸ is 1 to about 25 L-amino acid residues, 1 to about 20 L-amino acid residues, 1 to about 15 L-amino acid residues, 1 to about 11 L-amino acid residues, or 1 to about 5 L-amino acid residues.

Embodiment 43: The peptide according to Embodiment 42, wherein R⁸ is 1 to about 11 L-amino acid residues.

Embodiment 44: The peptide according to Embodiment 42 or Embodiment 43, wherein said L-amino acid residues comprises at least one amino acid residue that is N-glycosylated, O-glycosylated, C-glycosylated, S-glycosylated, or Se-glycosylated.

Embodiment 45: The peptide according to Embodiment 44, wherein said L-amino acid residues comprise at least one L-amino acid residue that is O-glycosylated.

Embodiment 46. The peptide according to Embodiment 45, wherein said amino acid residue that is O-glycosylated is an L-serine residue.

Embodiment 47: The peptide according to any one of Embodiments 1 to 6, 22 to 31, and 38 to 46, wherein R¹ and R² together form a bio-reversible moiety.

Embodiment 48: The peptide according to Embodiment 47, wherein said bio-reversible moiety is

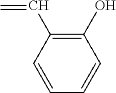

or ═N═N (azido moiety).

Embodiment 49: The peptide according to any one of Embodiments 1 to 6, 22 to 31, and 38 to 46, wherein one of R¹ or R² is hydrogen and one of R¹ or R² is —C(═O)OCH₂CH₃ or —C(═O)OCH₂OC(═O)CH₃.

Embodiment 50: The peptide according to any one of Embodiments 1 to 5, 7, 22 to 31, and 38 to 46, wherein R⁵ is a bio-reversible moiety.

Embodiment 51: The peptide according to Embodiment 50, wherein the bio-reversible moiety is —C(═O)CH₃.

Embodiment 52: The peptide according to Embodiment 1, selected from the group consisting of:
L-Phe-D-Val-L-Val-D-Phe (peptide 1a, Bilaid A);
L-Phe-D-Val-L-Val-D-Phe-NH₂ (peptide 1e);
L-Tyr-D-Val-L-Val-D-Phe (peptide 3a, Bilaid C);
L-Tyr-D-Val-L-Val-D-Phe-NH₂ (peptide 3b);
2,6-dimethyl-L-tyrosine-D-Val-L-Val-D-Phe-NH₂ (peptide 3c; Bilorphin);
2,6-dimethyl-L-tyrosine-D-Val-L-Val-D-Phe-L-Ser(β-Lac)-NH₂ (peptide 3g; Bilactorphin);
2,6-dimethyl-L-tyrosine-D-Val-L-Val-D-Phe-OCH₂CH₃ (peptide 4);
2,6-dimethyl-L-tyrosine-D-Va-L-Val-D-Phe-L-Pro-L-Asn-L-Leu-L-Aa-L-Glu-L-Lys-L-AIa-L-Leu-L-Lys-L-Ser-L-Leu-NH₂ (peptide 11);
2,6-dimethyl-L-tyrosine-D-Val-L-Val-D-Phe-NH₂ wherein the N-terminus is substituted with the bio-reversible moiety —C(═O)OCH₂OC(═O)CH₃ (peptide 10);
2,6-dimethyl-L-tyrosine-D-Val-L-Val-D-Phe-NH₂ wherein the N-terminus is substituted with the bio-reversible moiety

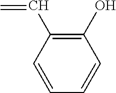

(peptide 8);
2,6-dimethyl-L-tyrosine-D-Val-L-Val-D-Phe-NH₂ wherein the hydroxy group on 2,6-dimethyl-L-tyrosine is substituted with the bio-reversible moiety —C(═O)CH₃ (peptide 5);
2,6-dimethyl-L-tyrosine-D-Val-L-Val-D-Phe-OCH₂CH₃ wherein the hydroxy group on 2,6-dimethyl-L-tyrosine is substituted with the bio-reversible moiety —C(═O)CH₃ (peptide 6);
2,6-dimethyl-L-tyrosine-D-Val-L-Val-D-Phe-NH₂ wherein the N-terminus is substituted with the bio-reversible moiety —C(═O)OCH₂CH₃ (peptide 7); and
2,6-dimethyl-L-tyrosine-D-Val-L-Val-D-Phe-NH₂ wherein the N-terminus is substituted with the bio-reversible moiety, ═N═N, to form an N-terminal azido group (peptide 9).

Embodiment 53: A peptide comprising
L-AA-L-Tyr-D-Val-L-Val-D-Phe-linker-sugar moiety;
L-AA-L-Tyr-D-Thr-L-Thr-D-Phe-linker-sugar moiety;
L-AA-L-Dmt-D-Val-L-Val-D-Phe-linker-sugar moiety;
L-AA-L-Dmt-D-Thr-L-Thr-D-Phe-linker-sugar moiety;
wherein L-AA is any L-amino acid residue optionally with at least one N-terminal —CH₃;
wherein the hydroxyl group of L-Tyr or L-Dmt is optionally alkylated; and
wherein the linker is preferably L-Ser or L-Thr.

Embodiment 54: The peptide according to any one of Embodiments 1 to 53, wherein said peptide, in comparison to morphine:
(a) exhibits a lower ratio of induction of C-terminal phosphorylation of MOPr versus G-protein activation; and/or
(b) exhibits a lower ratio of induction of β-arrestin recruitment versus G-protein activation; and/or
(c) exhibits a lower ratio of induction of MOPr internalisation versus G-protein activation.

Embodiment 55: The peptide according to any one of Embodiments 1 to 53, wherein said peptide, in comparison to morphine, exhibits a lower ratio of induction of β-arrestin recruitment versus G-protein activation.

Embodiment 56: The peptide according to any one of Embodiments 1 to 55, wherein said peptide exhibits an increase in inhibition of cAMP formation in comparison to vehicle at a concentration of about 10 μM in an assay using hMOPr.

Embodiment 57: The peptide according to any one of Embodiments 1 to 56, wherein said peptide in a competitive binding assay using [³H]DAMGO exhibits a Kᵢ of less than about 5 μM, less than about 3.5 μM, less than about 1 μM, less than about 0.8 μM, less than about 0.5 μM, or less than about 0.3 μM.

Embodiment 58: The peptide according to Embodiment 57, wherein said peptide exhibits a Kᵢ of less than about 0.5 μM or less than about 0.3 μM.

Embodiment 59: The peptide of any one of Embodiments 1 to 58, wherein said peptide crosses the blood brain barrier.

Embodiment 60: A pharmaceutical composition comprising a peptide according to any one of Embodiments 1 to 59 and at least one pharmaceutical excipient.

Embodiment 61: The pharmaceutical composition according to Embodiment 60, wherein said composition is formulated for oral administration.

Embodiment 62: The pharmaceutical composition according to Embodiment 60, wherein said peptide is glycosylated and said composition is formulated for oral administration, administration by injection, or intrathecal administration.

Embodiment 63: The pharmaceutical composition according to Embodiment 60, wherein said peptide is not glycosylated and said composition is formulated for nasal administration or intrathecal administration.

Embodiment 64: A method of treating pain comprising administering to a subject a peptide according to any one of Embodiments 1 to 59 or the pharmaceutical composition according to any one of Embodiments 60 to 63.

Embodiment 65: The method of Embodiment 64, wherein the pain the pain is post-operative pain, pain associated with nerve damage, pain associated with bone fracture, pain associated with a burn, or pain associated with a wound.

Embodiment 66: Use of a peptide according to any one of Embodiments 1 to 59 or the pharmaceutical composition according to any one of Embodiments 60 to 63 in the manufacture of a medicament for treating pain.

Embodiment 67: The use of Embodiment 66, wherein the pain the pain is post-operative pain, pain associated with nerve damage, pain associated with bone fracture, pain associated with a burn, or pain associated with a wound.

Embodiment 68: A peptide according to any one of Embodiments 1 to 59 for use in a method of treating pain.

Embodiment 69: A pharmaceutical composition according to any one of Embodiments 60 to 63 for use in a method of treating pain.

Embodiment 70: The peptide for use in the method of Embodiment 68 or the pharmaceutical composition for use in the method of Embodiment 69 wherein the pain is post-operative pain, pain associated with nerve damage, pain associated with bone fracture, pain associated with a burn, or pain associated with a wound.

Embodiment 71: A method of delivering analgesia comprising administering to a subject a peptide according to any one of Embodiments 1 to 59 or the pharmaceutical composition according to any one of Embodiments 60 to 63.

Embodiment 72: Use of peptide according to any one of Embodiments 1 to 59 or the pharmaceutical composition according to any one of Embodiments 60 to 63 for the manufacture of a medicament for delivering analgesia.

Embodiment 73: A peptide according to any one of Embodiments 1 to 59 or the pharmaceutical composition according to any one of Embodiments 60 to 63 for use in a method of delivering analgesia.

Embodiment 74: A method of treating pain or delivering analgesia with reduced adverse side effect(s), preferably reduced in comparison to morphine, comprising administering to a subject a peptide according to any one of Embodiments 1 to 59 or the pharmaceutical composition according to any one of Embodiments 60 to 63.

Embodiment 75: The method according to Embodiment 74, wherein said adverse side effect(s) is gastrointestinal (GI) inhibition and/or respiratory depression.

Embodiment 76: Use of peptide according to any one of Embodiments 1 to 59 or the pharmaceutical composition according to any one of Embodiments 60 to 63 for the manufacture of a medicament for a method of treating pain or delivering analgesia with reduced adverse side effect(s), preferably reduced in comparison to morphine.

Embodiment 77: The use according to Embodiment 76, wherein said adverse side effect(s) is gastrointestinal (GI) inhibition and/or respiratory depression.

Embodiment 78: A peptide according to any one of Embodiments 1 to 59 or the pharmaceutical composition according to any one of Embodiments 60 to 63 for use in a method of treating pain or delivering analgesia with reduced adverse side effect(s), preferably reduced in comparison to morphine.

Embodiment 79: The peptide for use in the method of Embodiment 78 or the pharmaceutical composition for use in the method of Embodiment 78, wherein said adverse side effect(s) is gastrointestinal (GI) inhibition and/or respiratory depression.

Embodiment 80: A peptide comprising Formula II

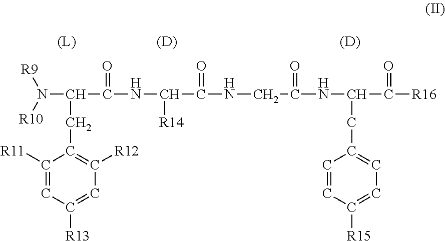

wherein, counting from the N-terminus, the first amino acid residue is an L-amino acid residue and the second and fourth amino acid residues are D-amino acid residues; wherein
R⁹ is hydrogen or a bio-reversible moiety optionally comprising a sugar moiety;
R¹⁰ is hydrogen or a bio-reversible moiety optionally comprising a sugar moiety;
wherein when one of R⁹ or R¹⁰ is a hydrogen and one of R⁹ or R¹⁰ is a bio-reversible moiety, the bio-reversible moiety is preferably —C(═O)OCH₂CH₃ or —C(═O)OCH₂OC(═O)CH₃;
wherein R⁹ and R¹⁰ may together form one bio-reversible moiety, wherein preferably the bio-reversible moiety is

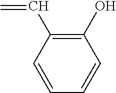

or ═N═N (azido moiety);
   R¹¹ and R¹² are independently selected from hydrogen or C₁-C₃ alkyl, preferably —CH₃;
   R¹³ is hydrogen, —OH, or a bio-reversible moiety optionally comprising a sugar moiety;
   R¹⁴ is a side chain of an amino acid or C₁-C₆ alkyl, preferably C₁-C₄ alkyl, more preferably —CH(CH₃)₂;
   R¹⁵ is hydrogen, —OH, or a bio-reversible moiety; and
   R¹⁶ is —OH, —O(C₁-C₃ alkyl), —NH₂,

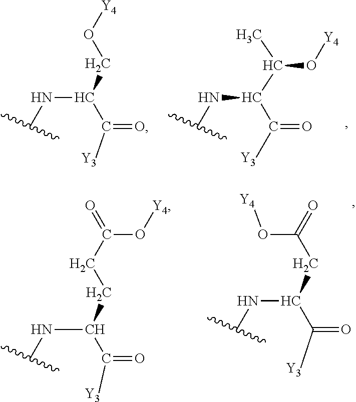

or 1 to about 30 L-amino acid residues;
Y₃ is —OH, —NH₂, or 1 to about 30 L-amino acid residues;
Y₄ is hydrogen or a sugar moiety, preferably a disaccharide moiety; and
wherein when R¹⁶ is 1 to about 30 L-amino acid residues (1) the L-amino acid residues are optionally residues that may be optionally glycosylated with a sugar moiety, preferably a disaccharide moiety, and (2) the C-terminus is optionally amidated.

Embodiment 81: The peptide according to Embodiment 80, wherein R¹⁴ is C₁-C₆ alkyl.

Embodiment 82: The peptide according to Embodiment 80 or Embodiment 81, wherein R¹⁴ is selected from the side chain of alanine, valine, norvaline, leucine, norleucine, or isoleucine.

Embodiment 83: The peptide according to any one of Embodiments 80 to 82, wherein R¹⁴ is a valine side chain (—CH(CH₃)₂).

Embodiment 84: The peptide according to Embodiment 80, wherein R¹⁴ is a threonine side chain.

Embodiment 85: The peptide according to any one of Embodiments 80 to 84, wherein R¹¹ and R¹² are —CH₃; and R¹³ is —OH.

Embodiment 86: The peptide according to any one of Embodiments 80 to 84, wherein R⁹ and R¹⁰ are each hydrogen.

Embodiment 87: The peptide according to any one of Embodiments 80 to 84, wherein R⁹, R¹⁰, R¹¹, R¹², and R¹³ are each hydrogen.

Embodiment 88: The peptide according to any one of Embodiments 80 to 84, wherein R⁹, R¹⁰, R¹¹, R¹², and R¹³ are hydrogen; R¹⁴ is C₁-C₄ alkyl; R¹⁵ is —OH.

Embodiment 89: A peptide comprising Formula II

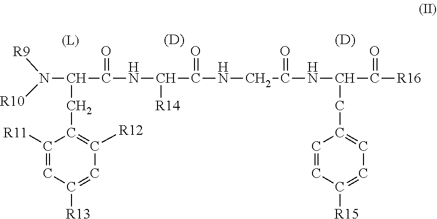

wherein, counting from the N-terminus, the first amino acid residue is an L-amino acid residue and the second and fourth amino acid residues are D-amino acid residues; wherein
R⁹ is hydrogen, a single bond, or —C₁-C₃ alkyl, preferably —CH₃;
R¹⁰ is hydrogen, a single bond, or —C₁-C₃ alkyl, preferably —CH₃;
R¹¹ and R¹² are independently selected from hydrogen or C₁-C₃ alkyl, preferably —CH₃;
R¹³ is hydrogen, —OH, or —O(C₁-C₃)alkyl;
R¹⁴ is a side chain of an amino acid or C₁-C₆ alkyl, preferably C₁-C₄ alkyl, more preferably —CH(CH₃)₂;
R¹⁵ is hydrogen, —OH, or a bio-reversible moiety; and
R¹⁶ is —OH, —O(C₁-C₃ alkyl), —NH₂,

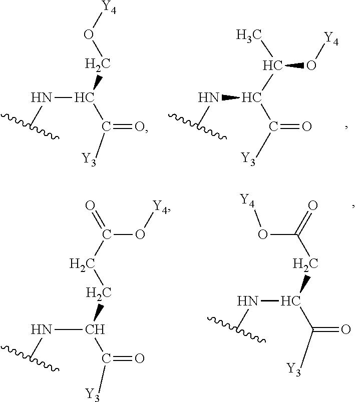

1 to about 30 L-amino acid residues, or a linker;
Y₃ is —OH, —NH₂, or 1 to about 30 L-amino acid residues;
Y₄ is hydrogen or a sugar moiety, preferably a disaccharide moiety;
   wherein when R¹⁶ is 1 to about 30 L-amino acid residues (1) the L-amino acid residues are optionally residues that may be optionally glycosylated with a sugar moiety, preferably a disaccharide moiety, and (2) the C-terminus is optionally amidated;
   wherein when R¹⁶ is a linker, the linker comprises a sugar moiety, preferably a disaccharide moiety such as lactose, and
   wherein when one of R⁹ or R¹⁰ is a single bond, one of R⁹ or R¹⁰ is hydrogen and the single bond is a peptide bond to an L-amino acid residue optionally N-terminally alkylated, preferably singly methylated.

Embodiment 90: The peptide according to Embodiment 89, wherein one of R⁹ and R¹ is hydrogen and one of R⁹ and R¹⁰ is —OH₃.

Embodiment 91: The peptide according to Embodiment 89 or Embodiment 90, wherein R¹³ is —O(C₁-C₃)alkyl, preferably —OCH₃.

Embodiment 92: The peptide according to Embodiment 91, wherein R¹¹ and R¹² are —OH₃.

Embodiment 93: The peptide according to Embodiment 91, wherein R¹¹ and R¹² are hydrogen.

Embodiment 94: The peptide according to Embodiment 89 or Embodiment 90, wherein R¹¹ and R¹² are —CH₃; and R¹³ is —OH.

Embodiment 95: The peptide according to Embodiment 89, wherein one of R⁹ or R¹⁰ is a single bond, one of R⁹ or R¹⁰ is hydrogen, and the single bond is a peptide bond to an L-amino acid residue.

Embodiment 96: The peptide according to Embodiment 95, wherein the L-amino acid residue has at least one N-terminal methylation.

Embodiment 97: The peptide according to Embodiment 95 or Embodiment 96, wherein the L-amino acid residue is an L-alanine residue.

Embodiment 98: The peptide according to any one of Embodiments 89 to 97, wherein R¹⁴ is a valine side chain (—CH(CH₃)₂).

Embodiment 99: The peptide according to any one of Embodiments 89 to 97, wherein R¹⁴ is threonine side chain.

Embodiment 100: The peptide according to any one of Embodiments 80 to 99, wherein R¹⁶ is —NH₂.

Embodiment 101: The peptide according to any one of Embodiments 80 to 99, wherein

R¹⁶ is

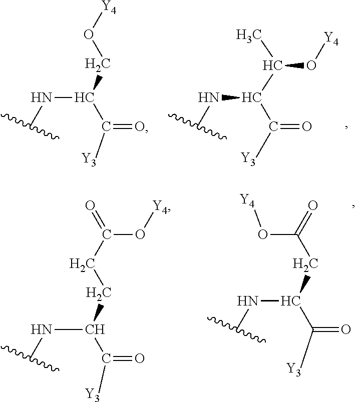

or 1 to about 30 L-amino acid residues;
Y₃ is —OH, —NH₂, or 1 to about 30 L-amino acid residues;
Y₄ is hydrogen or a sugar moiety, preferably a disaccharide moiety; and
wherein when R⁸ is 1 to about 30 L-amino acid residues (1) the L-amino acid residues are optionally residues that may be optionally glycosylated with a sugar moiety, preferably a disaccharide moiety, and (2) the C-terminus is optionally amidated.

Embodiment 102: The peptide according to Embodiment 101, wherein R¹⁶ is 1 to about 30 L-amino acids.

Embodiment 103: The peptide of Embodiment 102, wherein R¹⁶ is 1 to about 25 L-amino acid residues, 1 to about 20 L-amino acid residues, 1 to about 15 L-amino acid residues, 1 to about 11 L-amino acid residues, or 1 to about 5 L-amino acid residues.

Embodiment 104: The peptide according to Embodiment 103, wherein R¹⁶ is 1 to about 11 L-amino acid residues.

Embodiment 105: The peptide according to any one of Embodiments 102 to 104, wherein said L-amino acid residues comprises at least one amino acid residue that is N-glycosylated, O-glycosylated, C-glycosylated, S-glycosylated, or Se-glycosylated.

Embodiment 106: The peptide according to Embodiment 105, wherein said L-amino acid residues comprise at least one amino acid residue that is O-glycosylated.

Embodiment 107: The peptide according to Embodiment 106, wherein said amino acid residue that is O-glycosylated is an L-serine residue.

Embodiment 108: The peptide according to any one of Embodiments 89 to 99, wherein R¹⁶ is a linker.

Embodiment 109: The peptide according to Embodiment 108, wherein the linker comprises an amino acid based linker, peptide based linker, an amino acid comprising linker, and/or maleimide based linker, and/or a combination thereof.

Embodiment 110: The peptide according to Embodiment 101, wherein

### R¹⁶ is

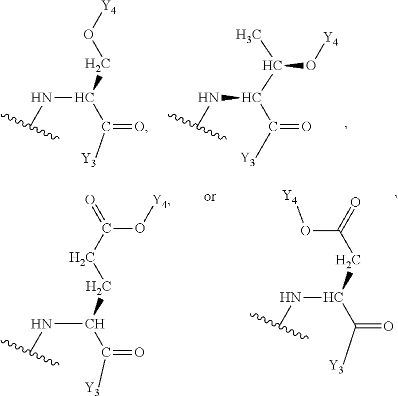 [0462] Y₃ is —OH, —NH₂, or 1 to about 30 L-amino acid residues; and
Y₄ is hydrogen or a sugar moiety, preferably a disaccharide moiety.

Embodiment 111: The peptide according to any one of Embodiments 80 to 99, wherein

### R¹⁶ is

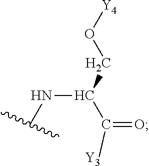

Y₃ is —OH or —NH₂; and Y₄ is hydrogen or a sugar moiety, preferably a disaccharide moiety.

Embodiment 112: The peptide according Embodiment 80, wherein R⁹, R¹⁰, R¹¹, R¹², and R¹³ are hydrogen; R¹⁴ is —CH(CH₃)₂; R¹⁵ is —OH; and
R¹⁶ is

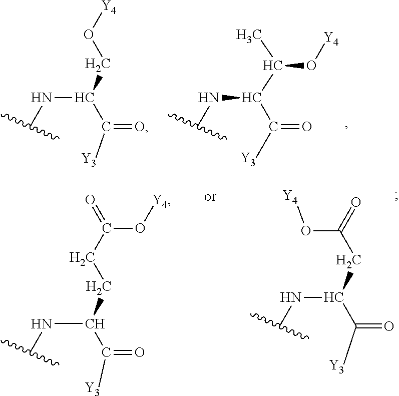

Y₃ is —OH or —NH₂; and Y₄ is hydrogen or a sugar moiety, preferably a disaccharide moiety.

Embodiment 113: The peptide according to any one of Embodiments 80 to 99, 101, 110, 111, and 112, wherein said sugar moiety is a disaccharide moiety, preferably wherein the disaccharide moiety is attached through a beta linkage.

Embodiment 114: The peptide according to Embodiment 113, wherein said disaccharide moiety is a lactose moiety or a melibiose moiety, preferably wherein the disaccharide moiety is attached through a beta linkage.

Embodiment 115: The peptide according to Embodiment 113 or Embodiment 114, wherein said disaccharide moiety is a lactose moiety, preferably wherein the lactose moiety is attached through a beta linkage.

Embodiment 116: The peptide according to any one of Embodiments 80 to 85, wherein R⁹ and R¹⁰ together form a bio-reversible moiety.

Embodiment 117: The peptide according to Embodiment 116, wherein said bio-reversible moiety is

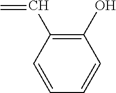

or ═N═N (azido moiety).

Embodiment 118: The peptide according to any one of Embodiments 80 to 85, wherein one of R⁹ or R¹⁰ is hydrogen and one of R⁹ or R¹⁰ is —C(═O)OCH₂CH₃ or —C(═O)OCH₂OC(═O)CH₃.

Embodiment 119: The peptide according to any one of Embodiments 80 to 85, wherein R¹³ is a bio-reversible moiety.

Embodiment 120: The peptide according to Embodiment 119, wherein the bio-reversible moiety is —C(═O)CH₃.

Embodiment 121: The peptide according to Embodiment 80 which is L-Phe-D-Val-Gly-D-Tyr-NH₂.

Embodiment 122: The peptide of any one of Embodiments 80 to 121 wherein said peptide in comparison to morphine:
(a) exhibits a lower ratio of induction of C-terminal phosphorylation of MOPr versus G-protein activation; and/or
(b) exhibits a lower ratio of induction of β-arrestin recruitment versus G-protein activation; and/or
(c) exhibits a lower ratio of induction of MOPr internalisation versus G-protein activation.

Embodiment 123: The peptide of any one of Embodiments 80 to 121, wherein said peptide, in comparison to morphine, exhibits a lower ratio of induction of β-arrestin recruitment versus G-protein activation.

Embodiment 124: The peptide of any one of Embodiments 80 to 123, wherein said peptide exhibits an increase in inhibition of cAMP formation in comparison to vehicle at a concentration of about 10 μM in an assay using hMOPr.

Embodiment 125: The peptide of any one of Embodiments 80 to 124, wherein said peptide in a competitive binding assay using [³H]DAMGO exhibits a Kᵢ of less than about 5 μM, less than about 3.5 μM, less than about 1 μM, less than about 0.8 μM, less than about 0.5 μM, or less than about 0.3 μM.

Embodiment 126: The peptide of Embodiment 125, wherein said peptide exhibits a Kᵢ of less than about 0.5 μM or less than about 0.3 μM.

Embodiment 127: The peptide of any one of Embodiments 80 to 126, wherein said peptide crosses the blood brain barrier.

Embodiment 128: A pharmaceutical composition comprising a peptide according to any one of Embodiments 80 to 127 and at least one pharmaceutical excipient.

Embodiment 129: The pharmaceutical composition according to Embodiment 128, wherein said composition is formulated for oral administration.

Embodiment 130: The pharmaceutical composition according to Embodiment 128, wherein said peptide is glycosylated and said composition is formulated for oral administration, administration by injection, or intrathecal administration.

Embodiment 131: The pharmaceutical composition according to Embodiment 128, wherein said peptide is not glycosylated and said composition is formulated for nasal administration or intrathecal administration.

Embodiment 132: A method of treating pain comprising administering to a subject a peptide according to any one of Embodiments 80 to 127 or the pharmaceutical composition according to any one of Embodiments 128 to 131.

Embodiment 133: The method of Embodiment 132, wherein the pain is post-operative pain, pain associated with nerve damage, pain associated with bone fracture, pain associated with a burn, or pain associated with a wound.

Embodiment 134: Use of a peptide according to any one of Embodiments 80 to 127 or the pharmaceutical composition according to any one of Embodiments 128 to 131 in the manufacture of a medicament for treating pain.

Embodiment 135: The use of Embodiment 134, wherein the pain is post-operative pain, pain associated with nerve damage, pain associated with bone fracture, pain associated with a burn, or pain associated with a wound.

Embodiment 136: A peptide according to any one of Embodiments 80 to 127 for use in a method of treating pain.

Embodiment 137: A pharmaceutical composition according to any one of Embodiments 128 to 131 for use in a method of treating pain.

Embodiment 138: The peptide for use in the method of Embodiment 136 or the pharmaceutical composition for use in the method of Embodiment 137 wherein the pain is post-operative pain, pain associated with nerve damage, pain associated with bone fracture, pain associated with a burn, or pain associated with a wound.

Embodiment 139: A method of delivering analgesia comprising administering to a subject a peptide according to any one of Embodiments 80 to 127 or the pharmaceutical composition according to any one of Embodiments 128 to 131.

Embodiment 140: Use of peptide according to any one of Embodiments 80 to 127 or the pharmaceutical composition according to any one of Embodiments 128 to 131 for the manufacture of a medicament for delivering analgesia.

Embodiment 141: A peptide according to any one of Embodiments 80 to 127 or the pharmaceutical composition according to any one of Embodiments 128 to 131 for use in a method of delivering analgesia.

Embodiment 142: A method of treating pain or delivering analgesia with reduced adverse side effect(s), preferably reduced in comparison to morphine, comprising administering to a subject a peptide according to any one of Embodiments 80 to 127 or the pharmaceutical composition according to any one of Embodiments 128 to 131.

Embodiment 143: The method according to Embodiment 142, wherein said adverse side effect(s) is gastrointestinal (GI) inhibition and/or respiratory depression.

Embodiment 144: Use of peptide according to any one of Embodiments 80 to 127 or the pharmaceutical composition according to any one of Embodiments 128 to 131 for the manufacture of a medicament for treating pain or delivering analgesia with reduced adverse side effect(s), preferably reduced in comparison to morphine.

Embodiment 145: The use according to Embodiment 144, wherein said adverse side effect(s) is gastrointestinal (GI) inhibition and/or respiratory depression.

Embodiment 146: A peptide according to any one of Embodiments 80 to 127 or the pharmaceutical composition according to any one of Embodiments 128 to 131 for use in a method of treating pain or delivering analgesia with reduced adverse side effect(s), preferably reduced in comparison to morphine.

Embodiment 147: The peptide for use in the method of Embodiment 146 or the pharmaceutical composition for use in the method of Embodiment 146, wherein said adverse side effect(s) is gastrointestinal (GI) inhibition and/or respiratory depression.

Embodiment 148: An isolated peptide comprising Formula III

X¹—X²—X³—X⁴ (III)

wherein:
X¹ is the N-terminal amino acid residue comprising an N-terminal moiety —NR¹⁷R¹⁸;
X⁴ comprises a C-terminal moiety selected from —C(═O)OH, —C(═O)O(C₁-C₃ alkyl), —C(═O)NH₂,

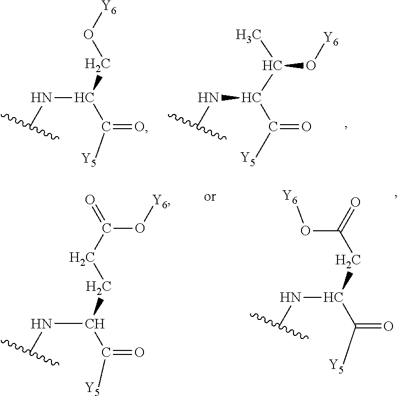

wherein Y₅ is —OH or —NH₂, and Y₆ is hydrogen or a sugar moiety, preferably a disaccharide moiety;
X¹ is an L-amino acid residue selected from L-tyrosine, 2,6-dimethyl-L-tyrosine, or L-phenylalanine, wherein when X¹ is L-tyrosine or 2,6-dimethyl-L-tyrosine, the residue is optionally O-substituted at the 4-position with a bio-reversible moiety optionally comprising a sugar moiety;
X² is a D-amino acid residue, preferably D-alanine, D-valine, D-leucine, or D-isoleucine, more preferably, D-valine;
X³ is glycine or an L-amino acid residue, wherein when X³ is an L-amino acid residue, X³ is preferably L-alanine, L-valine, L-leucine, or L-isoleucine, more preferably L-valine;
X⁴ is a D-amino acid residue selected from D-tyrosine or D-phenylalanine, wherein when X⁴ is D-tyrosine, the residue is optionally O-substituted with a bio-reversible moiety;
R¹⁷ and R¹⁸ are independently selected from hydrogen or a bio-reversible moiety optionally comprising a sugar moiety, or R¹⁷ and R¹⁸ together form a bio-reversible moiety optionally comprising a sugar moiety; and
wherein the peptide is a MOPr agonist.

Embodiment 149: The peptide according to Embodiment 148, wherein the C-terminal moiety is —C(═O)OH,

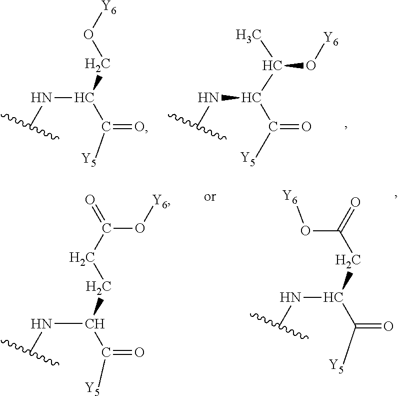

and Y₅ is —OH and Y₆ is hydrogen or a sugar moiety; the peptide further comprises about 5, 8, 11, 12, 20 or 26 additional L-amino acid residues on the C-terminus.

Embodiment 150: The peptide according to Embodiment 148 or Embodiment 149, wherein R¹⁷ and R¹⁸ are each hydrogen, X² is a D-valine residue, X³ is glycine or an L-valine residue, X⁴ comprises a C-terminal moiety selected from —C(═O)OH, —C(═O)NH₂,

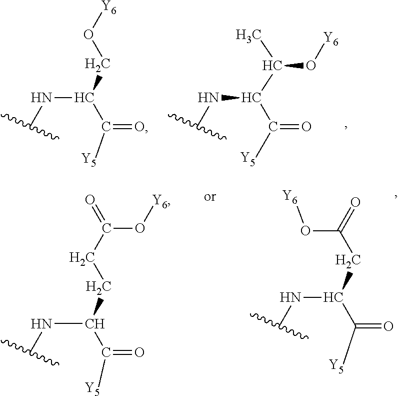

wherein Y₅ is —OH or —NH₂, and Y₆ is hydrogen or a sugar moiety, preferably a disaccharide moiety.

Embodiment 151: An isolated peptide comprising Formula III

X¹—X²—X³—X⁴ (III)

wherein:
X¹ is the N-terminal amino acid residue comprising an N-terminal moiety —NR¹⁷R¹⁸;
X⁴ comprises a C-terminal moiety selected from —C(═O)OH, —C(═O)O(C₁-C₃ alkyl), —C(═O)NH₂,

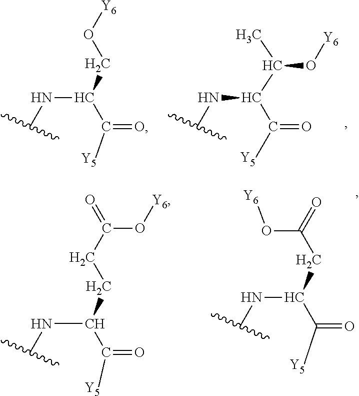

or a linker, wherein
Y₅ is —OH or —NH₂, and Y₆ is hydrogen or a sugar moiety, preferably a disaccharide moiety;
X¹ is an L-amino acid residue selected from L-tyrosine, 2,6-dimethyl-L-tyrosine, or L-phenylalanine, wherein when X¹ is L-tyrosine or 2,6-dimethyl-L-tyrosine, the residue is optionally O-substituted at the 4-position with C₁-C₃ alkyl;
X² is a D-amino acid residue, preferably D-threonine, D-alanine, D-valine, D-leucine, or D-isoleucine, more preferably, D-threonine or D-valine;
X³ is glycine or an L-amino acid residue, wherein when X³ is an L-amino acid residue, X³ is preferably L-threonine, L-alanine, L-valine, L-leucine, or L-isoleucine, more preferably L-threonine or L-valine;
X⁴ is a D-amino acid residue selected from D-tyrosine or D-phenylalanine, wherein when X⁴ is D-tyrosine, the residue is optionally O-substituted with a bio-reversible moiety;
R¹⁷ and R¹⁸ are independently selected from hydrogen, a single bond, or a —C₁-C₃ alkyl, preferably —CH₃; and
wherein when X⁴ comprises a linker, the linker comprises a sugar moiety, preferably a disaccharide moiety such as lactose,
wherein when one of R¹⁷ or R¹⁸ is a single bond, one of R¹⁷ or R¹⁸ is hydrogen and the single bond is a peptide bond to an L-amino acid residue that may optionally be N-terminally alkylated, preferably singly methylated; and
wherein the peptide is a MOPr agonist.

Embodiment 152: The peptide according to Embodiment 151, wherein the C-terminal moiety is —C(═O)OH,

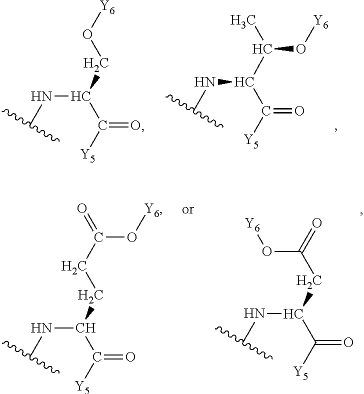

and Y₅ is —OH and Y₆ is hydrogen or a sugar moiety; the peptide further comprises a out 5, 8, 11, 12, 20 or 26 additional L-amino acid residues on the C-terminus.

Embodiment 153: The peptide according to Embodiment 151 or Embodiment 152, wherein X² is a D-valine residue, X³ is glycine or an L-valine residue, X⁴ comprises a C-terminal moiety selected from —C(═O)OH, —C(═O)NH₂,

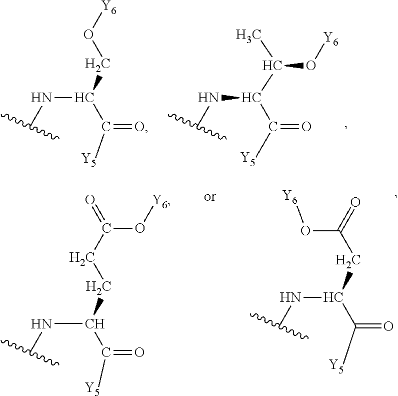

wherein Y₅ is —OH or —NH₂, and Y₆ is hydrogen or a sugar moiety, preferably a disaccharide moiety.

Embodiment 154: The peptide according to Embodiment 151 or Embodiment 152, wherein X² is a D-threonine residue, X³ is glycine or an L-threonine residue. X⁴ comprises a C-terminal moiety selected from —C(═O)OH, —C(═O)NH₂,

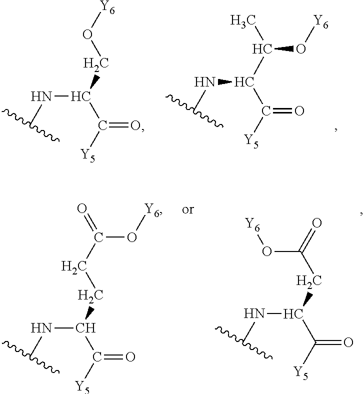

wherein Y₅ is —OH or —NH₂, and Y₆ is hydrogen or a sugar moiety, preferably a disaccharide moiety.

Embodiment 155: The peptide according to any one of Embodiments 151 to 154, wherein one of R¹⁷ and R¹⁸ is hydrogen and one of R¹⁷ and R¹⁸ is —CH₃.

Embodiment 156: The peptide according to any one of Embodiments 151 to 154, wherein one of R¹⁷ or R¹⁸ is a single bond, one of R¹⁷ or R¹⁸ is a hydrogen, and the single bond is a peptide bond to an L-amino acid residue.

Embodiment 157: The peptide according to Embodiment 156, wherein the L-amino acid residue has at least one N-terminal methylation.

Embodiment 158: The peptide according to Embodiment 156 or Embodiment 157, wherein the L-amino acid residue is an L-alanine residue.

Embodiment 159: The peptide according to Embodiment 151, wherein X⁴ comprises a linker.

Embodiment 160: The peptide according to Embodiment 154, wherein the linker comprises an amino acid based linker, peptide based linker, an amino acid comprising linker, and/or maleimide based linker, and/or a combination thereof.

Embodiment 161: The peptide according to any one of Embodiments 151 to 160, wherein X¹ is L-tyrosine or 2,6-dimethyl-L-tyrosine and wherein the L-tyrosine or 2,6-dimethyl-L-tyrosine is O-substituted at the 4-position with C₁-C₃ alkyl.

Embodiment 162: The peptide according to Embodiment 161, wherein X¹ is 2,6-dimethyl-L-tyrosine and wherein 2,6-dimethyl-L-tyrosine is O-substituted at the 4-position with C₁-C₃ alkyl

Embodiment 163: The peptide according to any one of Embodiments 148 to 162, wherein X⁴ comprises a C-terminal moiety selected from,

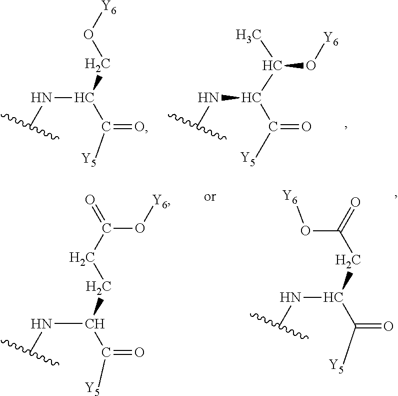

wherein Y₅ is —OH or —NH₂, and Y₆ is a disaccharide moiety, preferably wherein the disaccharide moiety is attached through a beta linkage.

Embodiment 164: The peptide according to Embodiment 162, wherein the disaccharide moiety is a lactose moiety, preferably wherein the lactose moiety is attached through a beta linkage.

Embodiment 165: The peptide according to Embodiment 149, wherein said additional L-amino acids comprise at least one amino acid residue that is N-glycosylated, O-glycosylated, C-glycosylated, S-glycosylated, or Se-glycosylated.

Embodiment 166: The peptide according to any one of Embodiments 148, 149, and 163 to 165, wherein R¹⁷ and R¹⁸ together form a bio-reversible moiety.

Embodiment 167: The peptide according to Embodiment 166, wherein said bio-reversible moiety is

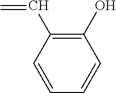

or ═N═N (azido moiety).

Embodiment 168: The peptide according to any one of Embodiments 148, 149 and 163 to 165, wherein one of R¹⁷ or R¹⁸ is hydrogen and one of R¹⁷ or R¹⁸ is —C(═O)OCH₂CH₃ or —C(═O)OCH₂OC(═O)CH₃.

Embodiment 169: The peptide according to Embodiment 148 selected from the group consisting of
L-Phe-D-Val-L-Val-D-Phe (peptide 1a, Bilaid A);
L-Phe-D-Val-L-Val-D-Phe-NH₂ (peptide 1e);
L-Tyr-D-Val-L-Val-D-Phe (peptide 3a, Bilaid C);
L-Tyr-D-Val-L-Val-D-Phe-NH₂ (peptide 3b);
2,6-dimethyl-L-tyrosine-D-Val-L-Val-D-Phe-NH₂ (peptide 3c; Bilorphin);
2,6-dimethyl-L-tyrosine-D-Val-L-Val-D-Phe-L-Ser(β-Lac)-NH₂ (peptide 3g; Bilactorphin);
L-Phe-D-Val-Gly-D-Tyr-NH₂ (peptide 2d);
2,6-dimethyl-L-tyrosine-D-Val-L-Val-D-Phe-OCH₂CH₃ (peptide 4);
2,6-dimethyl-L-tyrosine-D-Va-L-Val-D-Phe-L-Pro-L-Asn-L-Leu-L-Aa-L-Glu-L-Lys-L-AIa-L-Leu-L-Lys-L-Ser-L-Leu-NH₂ (peptide 11);
2,6-dimethyl-L-tyrosine-D-Val-L-Val-D-Phe-NH₂ wherein the N-terminus is substituted with the bio-reversible moiety —C(═O)OCH₂OC(═O)CH₃ (peptide 10);
2,6-dimethyl-L-tyrosine-D-Val-L-Val-D-Phe-NH₂ wherein the N-terminus is substituted with the bio-reversible moiety

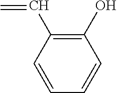

(peptide 8);
2,6-dimethyl-L-tyrosine-D-Val-L-Val-D-Phe-NH₂ wherein the hydroxy group on 2,6-dimethyl-L-tyrosine is substituted with the bio-reversible moiety —C(═O)CH₃ (peptide 5);
2,6-dimethyl-L-tyrosine-D-Val-L-Val-D-Phe-OCH₂CH₃ wherein the hydroxy group on 2,6-dimethyl-L-tyrosine is substituted with the bio-reversible moiety —C(═O)CH₃ (peptide 6);
2,6-dimethyl-L-tyrosine-D-Val-L-Val-D-Phe-NH₂ wherein the N-terminus is substituted with the bio-reversible moiety —C(═O)OCH₂CH₃ (peptide 7); and
2,6-dimethyl-L-tyrosine-D-Val-L-Val-D-Phe-NH₂ wherein the N-terminus is substituted with the bio-reversible moiety, ═N═N, to form an N-terminal azido group (peptide 9).

Embodiment 170: The peptide of any one of Embodiments 148 to 169, wherein said peptide in comparison to morphine:
(a) exhibits a lower ratio of induction of C-terminal phosphorylation of MOPr versus G-protein activation; and/or
(b) exhibits a lower ratio of induction of β-arrestin recruitment versus G-protein activation; and/or
(c) exhibits a lower ratio of induction of MOPr internalisation versus G-protein activation.

Embodiment 171: The peptide of any one of Embodiments 148 to 169 wherein said peptide, in comparison to morphine, exhibits a lower ratio of induction of β-arrestin recruitment versus G-protein activation.

Embodiment 172: The peptide of any one of Embodiments 148 to 171, wherein said peptide exhibits an increase in inhibition of cAMP formation in comparison to vehicle at a concentration of about 10 μM in an assay using hMOPr.

Embodiment 173: The peptide of any one of Embodiments 148 to 172, wherein said peptide in a competitive binding assay using [³H]DAMGO exhibits a Kᵢ of less than about 5 μM, less than about 3.5 μM, less than about 1 μM, less than about 0.8 μM, less than about 0.5 μM, or less than about 0.3 μM.

Embodiment 174: The peptide of Embodiment 173, wherein said peptide exhibits a Kᵢ of less than about 0.5 μM or less than about 0.3 μM.

Embodiment 175: The peptide of any one of Embodiments 148 to 174, wherein said peptide crosses the blood brain barrier.

Embodiment 176: A pharmaceutical composition comprising a peptide according to any one of Embodiments 148 to 175 and at least one pharmaceutical excipient.

Embodiment 177: The pharmaceutical composition according to Embodiment 176, wherein said composition is formulated for oral administration.

Embodiment 178: The pharmaceutical composition according to Embodiment 176, wherein said peptide is glycosylated and said composition is formulated for oral administration, administration by injection, or intrathecal administration.

Embodiment 179: The pharmaceutical composition according to Embodiment 176, wherein said peptide is not glycosylated and said composition is formulated for nasal administration or intrathecal administration.

Embodiment 180: A method of treating pain comprising administering to a subject a peptide according to any one of Embodiments 148 to 175 or the pharmaceutical composition according to any one of Embodiments 176 to 179.

Embodiment 181: The method of Embodiment 180, wherein the pain is post-operative pain, pain associated with nerve damage, pain associated with bone fracture, pain associated with a burn, or pain associated with a wound.

Embodiment 182: Use of a peptide according to any one of Embodiments 148 to 175 or the pharmaceutical composition according to any one of Embodiments 176 to 179 in the manufacture of a medicament for treating pain.

Embodiment 183: The use of Embodiment 182, wherein the pain is post-operative pain, pain associated with nerve damage, pain associated with bone fracture, pain associated with a burn, or pain associated with a wound.

Embodiment 184: A peptide according to any one of Embodiments 148 to 175 for use in a method of treating pain.

Embodiment 185: A pharmaceutical composition according to any one of Embodiments 176 to 179 for use in a method of treating pain.

Embodiment 186: The peptide for use in the method of Embodiment 184 or the pharmaceutical composition for use in the method of Embodiment 185, wherein the pain is post-operative pain, pain associated with nerve damage, pain associated with bone fracture, pain associated with a burn, or pain associated with a wound.

Embodiment 187: A method of delivering analgesia comprising administering to a subject a peptide according to any one of Embodiments 148 to 175 or the pharmaceutical composition according to any one of Embodiments 176 to 179.

Embodiment 188: Use of peptide according to any one of Embodiments 148 to 175 or the pharmaceutical composition according to any one of Embodiments 176 to 179 for the manufacture of a medicament for delivering analgesia.

Embodiment 189: A peptide according to any one of Embodiments 148 to 175 or the pharmaceutical composition according to any one of Embodiments 176 to 179 for use in a method of delivering analgesia.

Embodiment 190: A method of treating pain or delivering analgesia with reduced adverse side effect(s), preferably reduced in comparison to morphine, comprising administering to a subject a peptide according to any one of Embodiments 148 to 175 or the pharmaceutical composition according to any one of Embodiments 176 to 179.

Embodiment 191: The method according to Embodiment 190, wherein said adverse side effect(s) is gastrointestinal (GI) inhibition and/or respiratory depression.

Embodiment 192: Use of peptide according to any one of Embodiments 148 to 175 or the pharmaceutical composition according to any one of Embodiments 176 to 179 for the manufacture of a medicament for a method of treating pain or delivering analgesia with reduced adverse side effect(s), preferably reduced in comparison to morphine.

Embodiment 193: The use according to Embodiment 192, wherein said adverse side effect(s) is gastrointestinal (GI) inhibition and/or respiratory depression.

Embodiment 194: A peptide according to any one of Embodiments 148 to 175 or the pharmaceutical composition according to any one of Embodiments 176 to 179 for use in a method of treating pain or delivering analgesia with reduced adverse side effect(s), preferably reduced in comparison to morphine.

Embodiment 195: The peptide for use in the method of Embodiment 194 or the pharmaceutical composition for use in the method of Embodiment 194, wherein said adverse side effect(s) is gastrointestinal (GI) inhibition and/or respiratory depression.

Embodiment 196: A peptide according to any one of Embodiments 1 to 59, 80 to 127, and 148 to 175 for use in medicine.

Embodiments of the invention will now be described, by way of example, with reference to the accompanying drawings.

TABLE 1

| | | Sequence | | | | | |
|---|---|---|---|---|---|---|---|
| Name | # | (SEQ ID NO) | N- term | P1 | P2 | P3 | P4 |
| bilaid A | 1a | H-FvVf-OH | H | L-Phe | D-Val | L-Val | D-Phe |
| | | (SEQ ID NO: 1) | | | | | |
| | 1b | H-fVvF-OH | — | D-Phe | L-Val | D-Val | L-Phe |
| | | (SEQ ID NO: 4) | | | | | |
| | 1c | H-FVvf-OH | — | — | L-Val | D-Val | — |
| | | (SEQ ID NO: 5) | | | | | |
| | 1d | H-fvVF-OH | — | D-Phe | — | — | L-Phe |
| | | (SEQ ID NO: 6) | | | | | |
| | 1e | H-FvVf-NH [symbol] | — | — | — | — | — |
| | | (SEQ ID NO: 7) | | | | | |
| | 1f | H-fVvF-NH [symbol] | — | D-Phe | L-Val | D-Val | L-Phe |
| | | (SEQ ID NO: 8) | | | | | |
| | 1g | H-FVvf-NH [symbol] | — | — | L-Val | D-Val | — |
| | | (SEQ ID NO: 9) | | | | | |
| | 1h | H-FVVF-NH [symbol] | — | — | L-Val | — | L-Phe |
| | | (SEQ ID NO: 10) | | | | | |
| bilaid B | 2a | H-FvVy-OH | H | L-Phe | D-Val | L-Val | D-Tyr |
| | | (SEQ ID NO: 2) | | | | | |
| | 2b | H-FvVy-NH [symbol] | — | — | — | — | — |
| | | (SEQ ID NO: 11) | | | | | |
| | 2c | H-FVVY-NH [symbol] | — | — | L-Val | — | L-Tyr |
| | | (SEQ ID NO: 12) | | | | | |
| | 2d | H-FvGy-NH [symbol] | — | — | — | Gly | — |
| | | (SEQ ID NO: 13) | | | | | |
| | 2e | H-FGVy-NH [symbol] | — | — | Gly | — | — |
| | | (SEQ ID NO: 14) | | | | | |
| | 2f | H-FGGy-NH [symbol] | — | — | Gly | Gly | — |
| | | (SEQ ID NO: 15) | | | | | |
| bilaid C | 3a | H-YvVf-OH | H | L-Tvr | D-Val | L-Val | D-Phe |
| | | (SEQ ID NO: 3) | | | | | |
| | 3b | H-YvVf-NH [symbol] | — | — | — | — | — |
| | | (SEQ ID NO: 16) | | | | | |
| bilorphin | 3c | H-[Dmt]-vVf-NH [symbol] | — | Dmt | — | — | — |
| | | (SEQ ID NO: 17) | | | | | |
| | 3d | Aε-YvVf-NH [symbol] | CH [symbol] CO | — | — | — | — |
| | | (SEQ ID NO: 18) | | | | | |
| | 3e | H-YVVF-OH | — | — | L-Val | — | L-Phe |
| | | (SEQ ID NO: 19) | | | | | |
| | 3f | H-YVVF-NH [symbol] | — | — | L-Val | — | L-Phe |
| | | (SEQ ID NO: 20) | | | | | |
| Bilactorphin | 3g | H-[Dmt]-vVfS(β- Lac)-NH [symbol] | — | Dmt | — | — | — |
| | | (SEQ ID NO: 21) | | | | | |
| | 3h | H-[Dmt]-vVfS(β-D-Glc)-NH [symbol] | — | Dmt | — | — | — |
| | | (SEQ ID NO: 22) | | | | | |

| | | TetraQ, initial | | | | |
|---|---|---|---|---|---|---|
| | | screen | | | | |
| | | % inhibition of | | | | |
| | | forskolin- | | | | Agonist |
| | | stimulate cAMP | | | | potency |
| | | formation in | | Binding | | in LC |
| | | hMOPr HEK cells | | screen | | neurons |
| | | (10 um | CEREP | Ki (nM) | | EC50 |
| Name | C- term | DAMGO = 94%) | MOPr | DOPr | KOPr | (nM) |
| bilaid A | OH | 21% | 3,100 | | | |
| | — | 0 | | | | |
| | — | 0 | | | | |
| | — | 0 | | | | |
| | NH [symbol] | 47% | 750 | | | |
| | NH [symbol] | 0 | | | | |
| | NH [symbol] | 0 | | | | |
| | NH [symbol] | 19% | >10,000 | | | |
| bilaid B | OH | 0 | | | | |
| | NH [symbol] | 0 | | | | |
| | NH [symbol] | 0 | | | | |
| | NH [symbol] | 31% | 1600 | | | |
| | NH [symbol] | 0 | | | | |
| | NH [symbol] | 0 | | | | |
| bilaid C | OH | 77% | 210 | | | 4,200 |
| | NH [symbol] | | 93 | | | |
| bilorphin | NH [symbol] | | 1.1 | 190 | 770 | 130 |
| | NH [symbol] | | | | | >10,000 |
| | — | | | | | >10,000 |
| | NH [symbol] | | | | | >10,000 |
| Bilactorphin | L-Ser(β- | | | | | |
| | Lac)-NH [symbol] | | | | | |
| | L-Ser(β- | | | | | |
| | D-Glc)-NH [symbol] | | | | | |

Dmt = 2,6-dimethyl-L-tyrosine
Parent (native) peptide of each set highlighted
“—” indicates no change from parent peptide
where values are missing they were not determined [symbol]
indicates data missing or illegible when filed

### Example 1: Peptide Synthesis

The peptides in Table 1 were synthesized using Fmoc chemistry.

Novel Peptide Synthesis:

General synthetic details: Initial HPLC was performed on a system consisting of two Shimadzu LC-8A Preparative Liquid Chromatographs with static mixer, Shimadzu SPD-M10AVP Diode Array Detector and Shimadzu SCL-10AVP System Controller. Further HPLC was performed using an Agilent 1100 Series separations module equipped with Agilent 1100 Series diode array and/or multiple wavelength detectors, Polymer Laboratories PL-ELS1000 ELSD and Agilent 1100 Series fraction collector and running ChemStation (Revisions 9.03A or 10.0A). NMR spectra were acquired in DMSO-d₆ on a Bruker Avance 500 or a Bruker Avance 600 spectrometer under XWIN-NMR or Topspin control, referenced to residual ¹H signals. Electrospray ionisation mass spectra (ESIMS) were acquired using an Agilent 1100 series separations module equipped with an Agilent 1100 series LC/MSD mass detector and Agilent 1100 series diode array detector. High-resolution (HR) ESIMS measurements were obtained on a Finnigan MAT 900 XL-Trap instrument with a Finnigan API III source. Unless otherwise specified, a constant level of 0.1% TFA was used in all HPLC separations. Chiroptical measurements ([α]_{D}) were obtained on a Jasco P-1010 Intelligent Remote Module type polarimeter in a 100×2 mm cell.

Fmoc-L- and D-amino acids were obtained from Novabiochem (Laufelfingen, Switzerland) or Peptide Institute (Osaka, Japan). 2-Chlorotrityl chloride and Rinkamide resins were purchased from Novabiochem (Laufelfingen, Switzerland). 2-(1H-benzotriazol-1-yl)-1,1,3,3-tetramethyluronium hexafluorophosphate (HBTU) was obtained from Richelieu Biotechnologies (Quebec, Canada). Trifluoroacetic acid (TFA), N,N-diisopropylethylamine (DIEA) and N,N-dimethylformamide (DMF), all peptide synthesis grade, were purchased from Auspep (Melbourne, Australia).

Characterisation of Bilaids A-C:

bilaid A (FvVf-OH) (1a). light brown oil; [α]_{D}+9 (c 0.2, 0.1% TFA/MeOH); HRESI(+)MS m/z 533.2745 [(M+Na), C₂₈H₃₈N₄O₅Na requires 533.2740]; For ¹H, ¹³C and 2D NMR (600 MHz, DMSO-d₆) see FIG. 25 and Table 1.

bilaid B (FvVy-OH) (2a). light brown oil; HRESI(+)MS m/z 549.2692 [(M+Na)⁺, C₂₈H₃₈N₄O₆Na requires 549.2689]; For ¹H NMR (600 MHz, DMSO-d₆) see FIG. 25 and Table 1.

bilaid C (YvVf-OH) (3a). light brown oil; HRESI(+)MS m/z 527.2879 [(M+H)⁺, C₂₈H₃₉N₄O₆ requires 527.2870]; For ¹H NMR (600 MHz, DMSO-d₆) see FIG. 25 and Table 1.

General peptide synthesis procedure: All peptides were assembled manually by stepwise solid-phase peptide synthesis. 2-Chlorotrityl chloride resin (0.176 g, 0.25 mmol; for peptide acids) or Rink amide resin (0.176 g, 0.25 mmol; for peptide amides) was swollen in DMF for 2 h and drained. The first Fmoc protected amino acid (1 mmol) was dissolved in DMF (2 mL) and DIEA (174 μL, 1 mmol) was added. After complete dissolution of the amino acid, the mixture was added to the reaction vessel and shaken for 2 h. The resin was flow washed with DMF for 1 min. The Fmoc protecting group was removed by shaking the resin with 5% piperidine/DMF mixture (2×10 mL, each cycle for 1 min). After deprotection the resin was again flow washed for 1 min. The next amino acid (1 mmol) was activated with 0.5 M HBTU solution (2 mL) and DIEA (174 μL, 1 mmol) and was added to the reaction vessel. The mixture was shaken for 10 min and a ninhydrin test was performed to calculate the coupling yield. This test was repeated after each coupling. After completion of the assembly, the terminal Fmoc group was removed as described above, the resin washed with DMF followed by DCM and dried under nitrogen. The peptide was cleaved from the resin by shaking with 10 mL of cleavage mixture (TFA:water: 95:5) for 2 h. TFA was evaporated under N₂ gas.

Purification of synthetic peptide acids from solid phase synthesis: Reaction products for the synthetic peptides, were purified by preparative HPLC (Zorbax SB-C₁₈ column, 250×21.2 mm, 7 μm, isocratic 45% H₂O(0.1% TFA):MeOH for bilaid A (FvVf-OH, 35.3 mg) (1a) and fVvF-OH, 41.0 mg (1b); 30% H₂O(0.1% TFA):MeOH for FVvf-OH, 25.0 mg (1c) and fvVF-OH, 30.8 mg (1d); 40% H₂O(0.1% TFA); bilaid B (FvVy-OH, 44.8 mg) (2a) and bilaid C (YvVf-OH, 45.9 mg) (3a).

Solution conversion of tetrapeptide acids to amides: To a solution of di-tert-butyl-dicarbonate in 1,4-dioxane (29.3 mg/mL) was added pyridine (10.5 μL/mL) and ammonium bicarbonate (10.5 mg/mL). To 1a, 1b, 1c and 1d was added 300 μL of this solution and to 2a and 3a was added 600 μL, equating to 4 eq. After stirring for 3 d at 50° C., the reaction products were purified by preparative HPLC (Zorbax SB-C₈ 150×21.2 mm column; gradient of 90% H₂O(0.1% TFA):MeCN to MeCN over 15 min), to yield pure samples.

LC/MS analyses on synthetic peptides: Performed on Zorbax SB-C₈ 150×4.6 mm, 5 μm column (flow 1 mL/min; gradient 10-100% MeCN/H₂O (+isocratic 0.05% HCO₂H) over 15 min.

Chiral HPLC analysis on peptides: Performed on Astec Chirobiotic T column, 150×4.6 mm, 5 μm, 0.5 mL/min, isocratic MeOH (0.1% triethylamine, 0.2% AcOH, pH 6.23).

### N^{α}-(9-fIuorenylmethoxycarbonyl)-3-O-[2,3,6-tri-O-acetyl-4-O-(2,3,4,6-tetra-O-acetyl-β-D-galactopyranosyl)-β-D-glucopyranosyl]-L-serine (Fmoc-L-Ser[β-Lac(Ac₇)]—OH)

β-lactose peracetate was prepared according to the procedure outlined by Xu et al Journal of Carbohyrate Chemistry (2012) 31(9): 711-720. Briefly, α-lactose monohydrate (20.0 g) was added in portions to a stirring suspension of sodium acetate (5.0 g) in acetic anhydride (200 mL) with the temperature maintained at 135° C. After 1 h the solution was poured into ice-water (1 L) and stirred overnight. The resulting precipitate was collected by filtration, redissolved in CH₂Cl₂, washed with satd. NaHCO₃ and dried over MgSO₄. Following removal of solvent under reduced pressure it was crystallised from CH₂Cl₂/MeOH (16.1 g, 42%). ESI-MS (m/z): calc. 619.2 [M-OAc]⁺ found 619.3.

Fmoc-L-Ser-OH was O-β-lactosylated based on the procedure described by Salvador et al Tetrahedron (1995) 51(19):5643-5656. To a mixture of β-lactose peracetate (5.0 g) and Fmoc-L-Ser-OH (2.9 g) in anhydrous CH₂Cl₂ (100 mL) was added BF₃-Et₂O (2.8 mL) and stirred under nitrogen for 20 h. The solution was washed with 1 M HCl then water and dried over MgSO₄. Purified by silica gel chromatography (1% AcOH/2% MeOH in CH₂Cl₂) followed by RP-HPLC (50% B isocratic) (1.9 g, 27% from β-lactose peracetate). ESI-MS (m/z): calc. 946.3 [M+H]⁺ found 946.3.

### H-[2,6-dimethyl-L-tyrosyl]-D-Val-L-Val-D-Phe-L-Ser(β-Lac)-NH₂ (Bilactorphin, 3g)

Peptide assembly was performed using Fmoc chemistry on 0.5 mmol scale on Fmoc-Rink-amide polystyrene resin (substitution value 0.67 mmol/g). Fmoc deprotections were accomplished by treatments with 50% piperidine/DMF (2×1 min). Couplings were performed using three equivalents of Fmoc amino acid/HBTU/DIEA (1:1:1) relative to resin loading (30 min). N^{α}-Fmoc-O-β-lactosyl-L-serine was incorporated as the hepta-O-acetate (prepared as described above); N^{α}-Boc-2,6-dimethyl-L-tyrosine was used without side-chain protection. Cleavage from the resin and removal of side-chain protecting groups was achieved by treatment with 95% TFA/2.5% TIPS/2.5% H₂O for 2 h at room temperature. TFA was removed under a stream of nitrogen, and the product was precipitated using cold diethyl ether/n-hexane (1:1), washed with Et₂O, redissolved in 50% acetonitrile/0.1% TFA/H₂O and lyophilised. ESI-MS (m/z): calc. 1259.5 [M+H]+, found 1259.7. The crude product was deacetylated by treatment with a solution of 5% hydrazine/30% acetonitrile/H₂O for 5 h then purified by RP-HPLC (10 to 50% B over 40 min). (190 mg, 39% from initial resin loading). ESI-MS (m/z): calc. 965.5 [M+H]+, found 965.4.

General Materials and Methods

RP-HPLC solvent A was 0.05% TFA/H₂O and solvent B was 0.043% TFA/90% acetonitrile/H₂O. Analytical HPLC was performed on a Shimadzu LC20AT system using a Thermo Hypersil GOLD C18 2.1×100 mm column at flow rate of 0.3 mL/min. Absorbance was recorded at 214 nm. Preparative HPLC was performed on a Waters DeltaPrep 3000 system using a Vydac 208TP 50×250 mm column at a flow rate of 80 mL/min. Mass spectra were recorded in positive ionisation mode on an API 2000 triple quadrupole mass spectrometer (AB SCIEX, Framingham, Mass., USA). Fmoc amino acids and O-(1H-benzotriazol-1-yl)-1,1,3,3-tetramethyluronium hexafluorophosphate (HBTU) were from Iris Biotech (Marktredwitz, Germany), dimethylformamide (DMF) and diisopropylethylamine (DIEA) were from Auspep (Melbourne, Australia). Boc-2,6-dimethyl-L-tyrosine was purchased from AstaTech Inc (Bristol Pa., USA). All other reagents were obtained from Sigma Aldrich.

### Example 2: Inhibition of Forskolin Induced cAMP Formation

Procedure for measuring the level of intracellular cAMP produced upon modulation of adenylate cyclase activity by G-protein-coupled receptors (GPCRs). To test the functional interaction between opioids, and opioid-like compounds, and specific opioid GPCRs by measuring changes in intracellular cAMP levels relative to basal levels. The assay can measure agonist and antagonist activity on GPCRs by stimulating cells to either increase or decrease intracellular cAMP levels.

Materials

Reagents supplied in cAMP assay kit: Anti-cAMP acceptor beads, Streptavidin-coated donor beads, Biotinylated cAMP, cAMP standard, 3% Tween-20 solution. Additional reagents: 1 M HEPES (Muticel), 10% Tween-20 (Pierce), BSA (Sigma), 1×PBS (Gibco BRL), Sterile distilled water (Gibco BRL), Forskolin (Sigma), 10×HBSS (Hepes Buffered Salt Solution, Gibco BRL), IBMX solution (3-Isobutyl-1-Methylxanthine, Sigma), DMSO (Sigma), 95% Ethanol (Sigma), Complete growth media, Versene (Gibco). Equipment: Envision multilabel plate reader (Perkin Elmer), Optiplate-384 well plates (Perkin Elmer), TopSeal adhesive sealing film (Perkin Elmer), 96-well plates (Axigen, Polypropylene V bottom), Silicon 96-well plate sealing mats (Axigen), Single channel pipettors, Multichannel pipettor, Centrifuge, Vortex, 75 cm2 vented tissue culture flask, 50 ml conical tubes, 15 ml conical tubes, Electronic pipette aid, Disposable sterile transfer pipettes, haemoytometer, Microscope.

Method

The assay is performed in a 384-well plate and each data point is performed in triplicate. The test compounds should be analyzed on the same plate as controls and cAMP standards. The number of compounds to be screened for activity will determine the volume of reagents and cells required for each experiment. The test compounds, controls and cAMP standards can be added to the plate in advance or while the cells are incubating with stimulation buffer. The plate should be sealed with TopSeal adhesive sealing film to avoid evaporation.

Assay background: Detection of cAMP is based on the competition between intracellular cAMP and biotinylated cAMP linked streptavidin-coated donor beads for anti-cAMP conjugated acceptor beads. When the donor and acceptor beads are in close proximity a signal emitted at 520-620 nm is detected using the Envision multilabel plate reader.

Preparation of Reagents

### Stocks:

500 mM IBMX solution: Dissolve 100 mg IBMX in 900 μl DMSO to give a 500 mM stock solution. Aliquot and store at 20° C.
50 mM forskolin solution: Dissolve 5 mg forskolin in 244 μl of 95% ethanol to give a 50 mM stock solution. Store at 20° C. and use as required.
Fresh reagents-Prepare the following reagents fresh in 50 ml conical tubes:
Stimulation buffer (1×HBSS, 0.1% BSA, 1 mM IBMX): For 50 ml add 5 ml 10×HBSS to a 50 ml tube then make up to 50 ml with water. Add 50 mg BSA, place at 37° C. until BSA is dissolved then add 100 μl IBMX while the buffer is at 37° C. to ensure that the IBMX does not precipitate.
Lysis buffer (0.3% Tween-20, 5 mM HEPES, 0.1% BSA): For 40 ml add 1.2 ml 10% Tween-20 and 200 μl 1 M HEPES to a 50 ml tube then make up to 40 ml with water. Add 50 mg BSA, place at 37° C. until BSA is dissolved.
Stimulation buffer with forskolin (*200 μM in stimulation buffer): Add forskolin to a dilution of 1:250 from the 50 mM stock to the required amount of stimulation buffer. It should be noted that the final concentration in the assay plate will be halved. *The optimal concentration of forskolin in the assay is cell line specific and should be optimized.

Preparation of test peptides: Test peptides are typically tested at a final concentration of 10 μM. As most test peptides are dissolved in 100% DMSO it is recommended that the DMSO concentration is limited to 2% v/v during cell stimulation to ensure maximum cell viability and responsiveness. Prepare 1 mM and 100 μM stock solutions of test peptides in an appropriate diluent and store at 4° C. For a library of peptides this can be performed in 96 well plates sealed with silicon sealing mats. Test peptides are diluted fresh from stock solutions to a working concentration in stimulation buffer with forskolin. It should be noted that as peptides are diluted 1:1 with cells in the assay the working concentration should be twice the final required concentration. It may be necessary to perform assays on compounds freshly diluted from DMSO stocks compounds to avoid experimental variability.

Control compounds: Compounds with known activity are used as controls. Controls are typically used at 1 μM and are diluted fresh in stimulation buffer with forskolin from stocks stored at −20° C. For the assay 5 μl of prepared control compounds are added per well in triplicate.

Preparation of cells: For best results cells should be low passage at 70-90% confluence. To prepare cells for the assay remove growth medium, add Versene and then incubate at 37° C. for approximately 5 minutes to allow cells to detach from the tissue culture plastic. Collect cells and centrifuge for 2 minutes at 275×g. Decant the supernatant and resuspend the cell pellet in 1×PBS. Determine the cell concentration using a haemocytometer. Re-centrifuge cells for 2 minutes at 275×g and decant supernatant. Resuspend cells in stimulation buffer to a final concentration of 1-4×10⁴ cells per ml. Note that the cells number will influence the cAMP levels prevailing before (basal) and after adenylate cyclase activation. A cell titration should be performed to optimize the difference between basal and stimulation levels of cAMP. Cells are incubated in stimulation buffer for 20 to 30 minutes at 37° C. prior to adding 5 μl to wells containing test and control compounds. Note that cells are not added to the cAMP standards.

Preparation of cAMP standard curve: Prepare a standard cAMP dilution series from the kit supplied 50 μM cAMP solution. Vortex at maximum intensity for 5 seconds before use. Serially dilute to provide a final concentration range from 5 μM to 0.5 nM cAMP (for example: 5 μM, 0.5 μM, 50 nM, 15 nM, 5 nM, 1.5 nM, and 0.5 nM cAMP). A positive control (no cAMP) should also be included. For the assay 10 μl of standard dilutions are added per well in triplicate.

Preparation of acceptor and donor bead solutions: The anti-cAMP conjugated acceptor beads and streptavidin-coated donor beads are light sensitive and should be handled under subdued lighting or under lights fitted with green filters. Once the beads have been added to the assay plate it should be wrapped in foil so that incubations are performed in the dark. Prepare acceptor and donor bead solutions in 15 ml conical tubes while the cells are incubating and keep in the dark until required. For the acceptor bead solution gently mix 10 μl bead suspension per ml of lysis buffer. For the donor bead solution use 10 μl bead suspension per ml of lysis buffer and 0.75 μl/ml of cAMP-biotin and mix gently.

cAMP Assay Procedure

1. Add standards (10 μl/well), control compounds (5 μl/well) and test peptides (5 μl/well) into 384-well plates and seal with Top Seal adhesive sealing film. Leave at room temperature until the cell incubation is complete.
2. Add 5 μl cells incubated in stimulation buffer to the wells containing test peptides and control compounds, but not to the standards. Incubate cells and compounds for 30 min at 37° C.
3. Add 10 μl lysis buffer per well.
4. Under subdued lighting add 5 μl acceptor bead solution to each well. Wrap plate in foil and incubate at room temperature with gentle mixing on an orbital shaker for 60 min.
5. Also under subdued lighting add 5 μl donor bead solution to each well, wrap the plate in foil, and then incubate overnight at room temperature with gentle mixing on an orbital shaker.
6. Measure cAMP levels on Envision multilabel plate reader.

Results analysis: Analyse results using PRISM software to calculate the intracellular levels of cAMP for each triplicate data point and the standard deviation of these data points.

In accordance with the method described above, the peptides were screened for inhibition of forskolin induced cAMP formation in HEK cells expressing the human MOPr (hMOPr).

Neither Bilaid B (2a) nor its C-terminal carboxamide analogue (2b) showed activity at 10 μM, indicating the phenylalanine at the fourth amino acid position is more favourable.

As shown in Table 1, bilaid A (peptide 1a, H-FvVf-OH) and bilaid C (peptide 3a, H-YvVf-OH) showed activity at 10 μM. In contrast, analogues of bilaid A having DLDL (1b) stereochemistry or LLDD (1c) stereochemistry were inactive at 10 μM and an analogue of bilaid A having LLLL stereochemistry (1h) was less active at 10 μM, highlighting the importance of the LDLD motif for maintaining MOPr activity.

### Example 3: Competitive Binding Assay (MOPr)

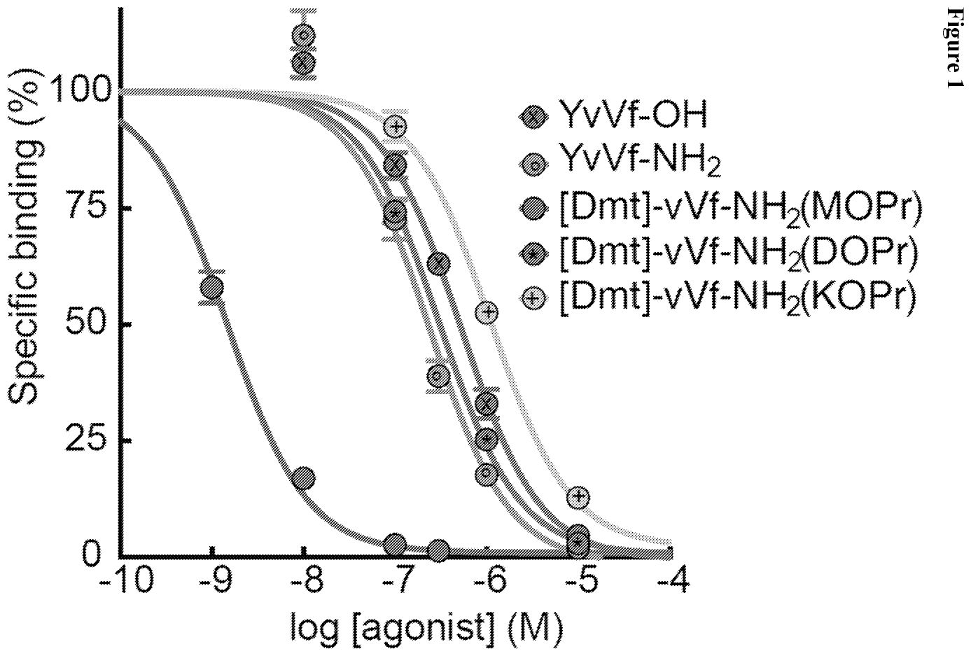

Peptides were tested for competitive binding to hMOPr against the MOPr agonist [³H]DAMGO. Bilaid A (1a) showed modest affinity (Kᵢ=3.1 μM) that was improved 4-fold through C-terminal amidation (1e, Kᵢ=0.75 μM). The N-terminal tyrosine containing Bilaid C (3a) displayed sub-micromolar binding (Kᵢ=210 nM) at the hMOPr. C-terminal amidation (3b) doubled MOPr affinity (Kᵢ=93 nM), consistent with the increased binding of 1e over 1a. Dimethylation of the N-terminal tyrosine ([Dmt]-vVf-NH₂ (3c)) (Dmt=2,6-dimethyl-L-tyrosine) see Zhao et al J Pharmacol Exp Ther (2003) 307(3): 947-54) resulted in a further increase to yield a Kᵢ of 1.1 nM. Peptide 3c (designated bilorphin), bound with nearly 200-fold selectivity for hMOPr over hDOPr (Kᵢ=190 nM) and 700-fold selectivity over hKOPr (Kᵢ=770 nM). See FIG. 1 and Table 1.

Competitive MOPr binding was determined using a filtration separation followed by liquid scintillation counting procedure after incubation of membranes prepared from human recombinant MOPr expressed in HEK-293 cells (Human Embryonic Kidney cell line) with [³H]DAMGO (0.5 nM) plus various concentrations of unlabelled peptides for 120 minutes at 22° C. The specific ligand binding to the receptors is defined as the difference between the total binding and the nonspecific binding determined in the presence of an excess of an unlabelled opioid ligand (naloxone, 10 μM). The results are expressed as a percent of control specific binding ((measured specific binding/control specific binding)×100) obtained in the presence of unlabelled peptides of interest. The IC₅₀ values (concentration causing a half-maximal inhibition of control specific binding) and Hill coefficients (nH) were determined by non-linear regression analysis of the competition curves generated with mean replicate values using Hill equation curve fitting (Y=D+[(A−D)/(1+(C/C₅₀nH)], where Y=specific binding, D=minimum specific binding, A=maximum specific binding, C=compound concentration, C₅₀=IC₅₀, and nH=slope factor).

### Example 4: Competitive Binding Assay (DOPr)

Peptides were tested for competitive binding to hDOPr against the DOPr agonist [³H]DADLE. See FIG. 1 and Table 1.

Inhibition of binding to human recombinant DOPr (hDOPr) expressed in CHO (Chinese Hamster Ovary) cell line was performed as described for MOPr binding (Example 3) except that incubation was in [3H]DADLE (0.5 nM) for 120 min at 22° C.

### Example 5: Competitive Binding Assay (KOPr)

Peptides were tested for competitive binding to hKOPr against the KOPr agonist [³H]U69593. See FIG. 1 and Table 1.

Inhibition of binding to human recombinant KOPr (hKOPr) expressed in CHO (Chinese Hamster Ovary) cell line was performed as described for MOPr binding (Example 3) except that incubation was in [³H]U69593 (2 nM) for 60 min at 22° C.

### Example 6: Patch Clamp Recordings of Activated G-Protein

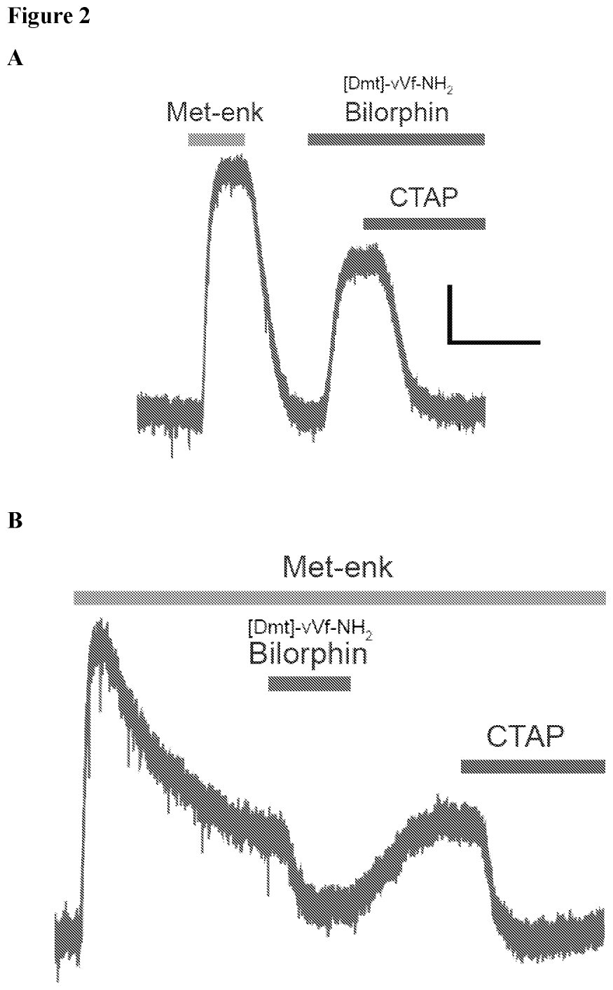

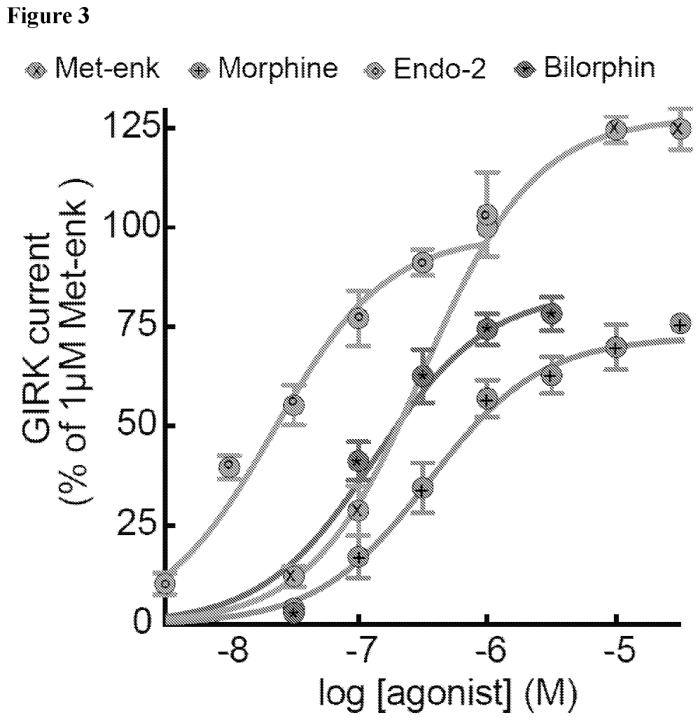

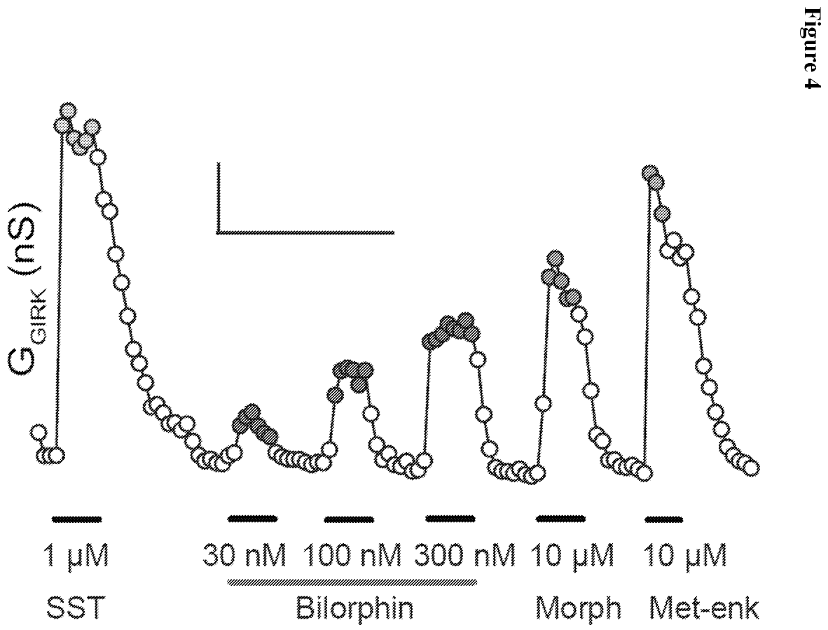

To assess functional activity of bilorphin, patch-clamp recordings of G-protein activated, inwardly rectifying potassium channel (GIRK) currents were made in rat locus coeruleus (LC) neurons, which natively express MOPr but not DOPr or KOPr (North et al Proc Natl Acad Sci USA. (1987) 84(15):5487-91). Bilorphin acted as an agonist with potency greater than morphine. Its actions were completely reversed by the highly MOPr selective antagonist CTAP (n=9, FIG. 2A; Table 1) (Pelton et al J Med Chem (1986) 29(11):2370-5) establishing that bilorphin does not act on closely related receptors expressed by LC neurons such as NOPr or somatostatin receptors (Connor et al Br J Pharmacol (1996) 119(8): 1614-8). Bilaid C analogues with an acetylated N-terminal (3d), or all L-stereoisomers (3e and 3f), were inactive in LC neurons at 10-30 μM, indicating the importance of a free N-terminal and the native LDLD motif for maintaining MOPr activity (Table 1). The submaximal, partial agonist-like action of bilorphin in LC neurons was confirmed by its partial antagonism of the full agonist actions of met-enkephalin. This was tested after MOPr was desensitized for 10 min to produce a stable activated response after acute desensitization, as well as to reduce functional receptor reserve (n=12, FIG. 2B).

Brain slice electrophysiology: Brain slices containing locus coeruleus LC neurons were prepared from male Sprague Dawley rats (4-6 weeks) as described previously (Sadeghi M et al Br J Pharmacol (2015) 172(2):460-8, incorporated by reference). Briefly, rats were anesthetized with isoflurane and decapitated. The brain was dissected and mounted in a vibratome chamber (Leica biosystem, VT100, Wetzlar, Germany) in order to prepare horizontal brain slices (280 μm). Slices were cut and stored in warm (34° C.) artificial cerebrospinal fluid (ACSF) containing the following (in mM): 125 NaCl, 2.5 KCl, 2 CaCl₂, 1 MgCl₂, 1.25 NaH₂PO₄, 25 NaHCO₃ and 11 glucose supplemented with 0.01 (+) MK801 (95% O₂-5% CO₂). Slices were incubated in warm oxygenated ACSF for at least 30 min before recording. Slices were transferred to the recording chamber while warm ACSF (34° C.) was superfused at a rate of ˜2 mL/min. Whole-cell voltage-clamp recordings were acquired from LC neurons with Multiclamp 700B amplifier (Molecular Devices, CA, USA) at holding potential of −60 mV. Recording pipettes (2-4 MΩ) were filled with internal solution containing (in mM): 135 potassium gluconate, 8 NaCl, 10 HEPES, 0.5 EGTA, 2 Mg-ATP and 0.3 Na-GTP; pH 7.3, 280-285 mOsM. Continuous current recordings were collected in chart mode at 500 Hz and filtered at 20-50 Hz using Axograph X (Axograph Scientific, Sydney, Australia). Series resistance monitored throughout the experiments and remained <15 MΩ; otherwise the data were discarded. Outward current was measured as the difference between baseline current and peak current of drug application.

### Example 7: Assessment of Relative Potency and Intrinsic Efficacy

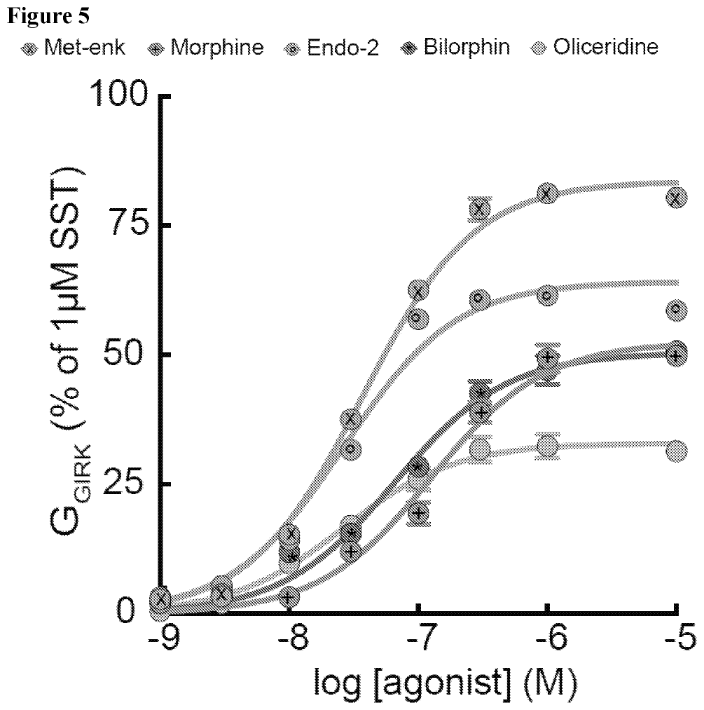

The relative potency and intrinsic efficacy (maximum response) of bilorphin also in other signalling pathways was examined in AtT20 cells stably expressing FLAG tagged mouse MOPr (mMOPr) to enable analysis of bias (FIG. 5). To reliably determine the relative intrinsic efficacy of bilorphin to activate G-proteins (G_{GIRK}), it was ensured that no MOPr agonist reached a ceiling effect. β-chlornaltrexamine was used to irreversibly inactivate a sufficient fraction of receptors to reduce the maximum response of met-enkephalin to 80% of that produced by a supramaximal concentration of somatostatin acting on native SST receptors in the same cells (FIGS. 4, 5). Maximal activation of SST receptors normally produces a G_{GIRK} increase equivalent to a maximal activation of MOPr (Yousuf et al Mol Pharmacol (2015) 88(4): 825-35). Under these conditions, bilorphin, morphine and endomorphin-2 all displayed similar maximal responses indicating they have similar intrinsic efficacies. With the exception of met-enkephalin all agonists displayed similar potencies in brain slices and the cell line. The reduced potency of Met-enkephalin in brain slices is known to result from its degradation by enkephalinases and other peptidases (Williams, et al. J. Pharmac. Exp. Ther. (1987) 243: 397-401.) As expected from brain slice experiments all three opioids were moderately efficacious but less so than Met-enkephalin (FIGS. 5, 23A). In contrast, the G-protein biased, small molecule agonist oliceridine, activated G_{GIRK} significantly less efficaciously than either morphine or bilorphin (FIGS. 5, 23A).

Expression of MOPr in AtT20 Cells:

Wild type mMOPr was cloned in pcDNA3.1 plasmids with FLAG-tag and expressed stably in AtT20 cells at a deliberately low level of expression (8 μmol/mg protein; 2×10⁵ receptors/cell estimated from cytometry), as previously described Borgland et al J Biol Chem (2003) 278(21): 18775-84. For patch clamp experiments AtT20 cells were seeded on 35-mm polystyrene culture dishes (Beckton, Dickinson Biosciences) in Dulbecco modified Eagle medium (Gibco, Life Technologies, Australia) containing 4.5 g/L glucose, penicillin-streptomycin (100 μl/ml), G418 (50 mg/ml.) (Gibco, Invitrogen) and 10% FBS. Cell cultures were kept in humidified 5% CO₂ atmosphere at 37° C. Cells were ready for recording after 24 hours incubation.

Cultured cell electrophysiology: Perforated patch clamp recordings were performed as previously described (Yousuf et al. Mol Pharmacol. (2015) 88(4):825-35). Pipettes were pulled from borosilicate glass (AM Systems, Everett, Wash., USA) yielding input resistances between 3.5-4.5 MΩ and were filled with internal solution containing 135 mM potassium gluconate, 3 mM MgCl₂, 10 mM HEPES (adjusted to pH 7.4 with KOH). The recording electrodes were first front filled with this internal solution and then backfilled with the same solution containing 200 μg/ml amphotericin B (in 0.8% DMSO). For measuring I_{GIRK} the KCl concentration in the bath was increased to 20 mM (substituted for NaCl) before the start of the measurements and was maintained throughout the experiments as previously described in Yousuf et al. (2015). Liquid junction potential was calculated to be +16 mV and was adjusted before the start of each recording. Currents were recorded at 37° C. in a fully enclosed, temperature-controlled recording chamber using an Axopatch 200B amplifier and pCLAMP 9.2 software, and digitized using Digidata 1320 (Axon Instruments, Molecular Devices, Sunnyvale, Calif., USA). Currents were sampled at 100 Hz, low pass filtered at 50 Hz and recorded on hard disk for later analyses. I_{GIRK} was recorded using a 200 ms voltage step to −120 mV from a holding potential of −60 mV delivered every 2 s. Drugs were perfused directly onto cells using a ValveLink 8.2 pressurized pinch valve perfusion system (AutoMate Scientific, USA).

Data Analyses. All data are shown as the mean±SEM and analysed using GraphPad Prism v7. All data points are plotted as chord GIRK conductance (G_{GIRK}, nS) using the following calculation: [I_{GIRK} (−60 mV)−I_{GIRK} (−120 mV)]pA/60 mV.

Assessment of Inducing C-Terminal Phosphorylation

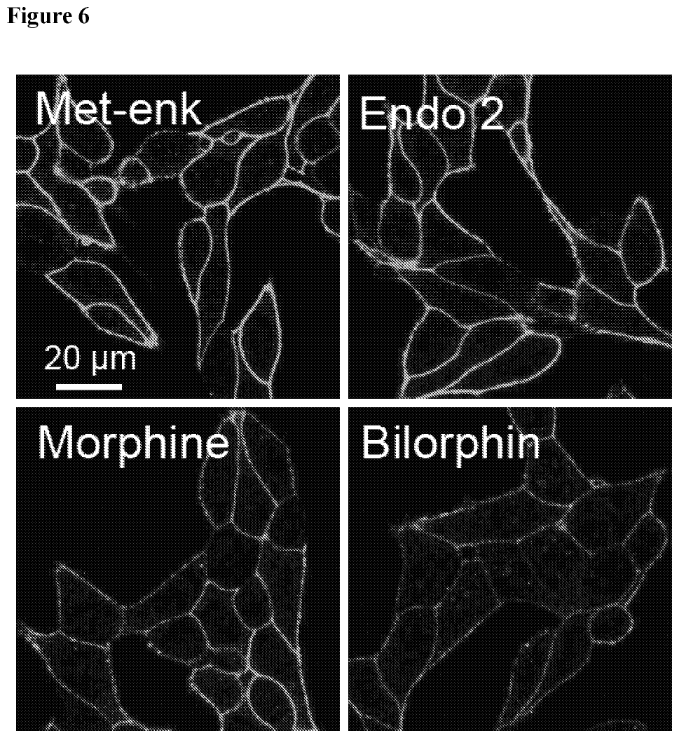

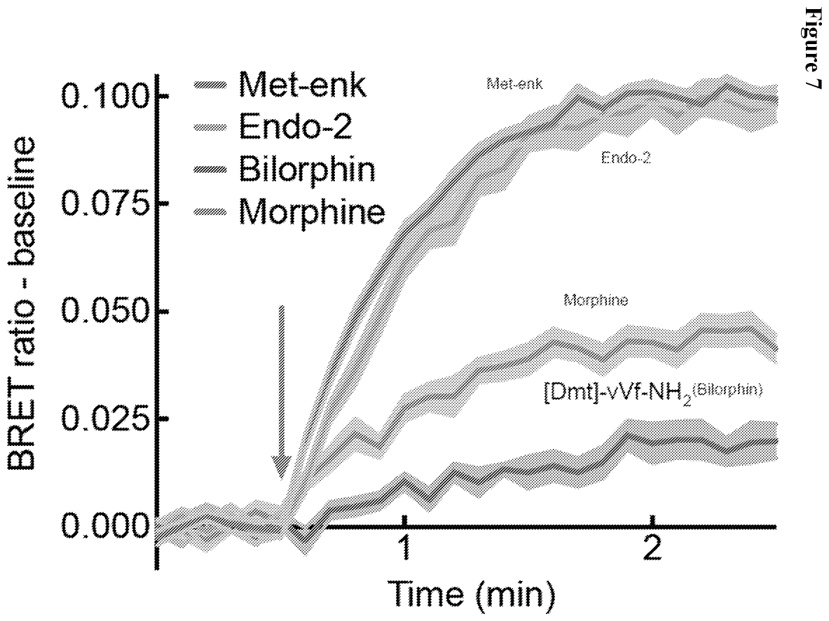

MOPr C-terminal phosphorylation, β-arrestin recruitment and internalisation are thought to contribute to on-target opioid analgesic side effects so that G-protein biased opioids that avoid arrestin signalling should show an improved side effect profile (Manglik et al Nature (2016) 537(7619):185-190; Schmid et al Cell (2017) 171(5):1165-1175; DeWire et al J Pharmacol Exp Ther (2013) 344(3):708-17). Agonist-induced phosphorylation of residue serine 375 (Ser³⁷⁵) drives β-arrestin recruitment and internalisation (El Kouhen et al J Biol Chem (2001) 276(16): 12774-80). The activity of bilorphin for inducing C-terminal phosphorylation, β-arrestin recruitment and MOPr internalisation was assayed in the same AtT20 cell line used to determine G-protein activation. The activity of bilorphin-induced Ser³⁷⁵ phosphorylation was determined using a phosphosite specific antibody (FIGS. 6, 9; Doll et al Br J Pharmacol (2011) 164(2): 298-307). Surprisingly, and unlike other opioid peptides (Thompson et al Biochem Pharmacol (2016) 113:70-87), bilorphin produced very low levels of pSer375 immunoreactivity at saturating concentrations (30 μM, FIGS. 6, 9, 23B). Maximal phosphorylation by bilorphin was similar to and displayed a trend to be less than that produced by morphine, which is known to only weakly phosphorylate MOPr at Ser³⁷⁵ (FIG. 23) (McPherson et al (2010) Mol Pharmacol 78: 756-766).

Ser³⁷⁵ Phosphorylation assay: AtT20 cells stably expressing MOPr were grown on coverslip to ˜50% confluence. Cells were serum starved for at least 30 min and then incubated in the absence or presence of the indicated ligand for 5-10 min at 37° C. Phosphorylation was terminated by fixing the cells with −30° C. methanol followed by 10 min incubation on ice. Cells were washed three times with phosphate buffered saline (PBS) and then heated in sodium citrate buffer (10 mM, 0.05% Tween 20, pH.6) for 20 min at 95° C. Cells were incubated with anti-phospho Ser³⁷⁵ antibody (1:200, Cell Signalling) overnight at room temperature. Next day, labeled receptors were stained with Alexa-fluor 488 antibody (1 μg/ml, 1 h at room temperature, Thermo Fisher Scientific). Imaging was performed as detailed below.

Imaging: Images of receptor phosphorylation and internalization (Example 10) were acquired using Zeiss 510 Meta laser scanning confocal microscope at a resolution of 1024×1024 pixels using a 60× oil emulsion objective. Imaging parameters including laser intensity, photomultiplier tube (PMT) voltage and offset remained constant for each experiment. Mean fluorescence intensity was measured using ImageJ software to calculate mean gray value of an area defined outside a single cell. Each experiment was normalized to the mean of untreated cells as 0% and the mean of cells treated with saturating concentrations of Met-enk (30 μM) as 100%.

### Assessment of β-Arrestin 2 Recruitment

Using MOPr-luciferease and β-arrestin2-YFP constructs, a BRET assay was performed to determine β-arrestin 2 recruitment to the receptor (FIGS. 7, 9) (Thompson et al Biochem Pharmacol (2016) 113:70-87). Similar to Ser³⁷⁵ phosphorylation, saturating concentrations of bilorphin induced very low levels of BRET efficiency relative to known agonists, and was also significantly less than morphine (up to 30 μM) (FIG. 7).

Arrestin recruitment: Agonist-induced recruitment of β-arrestin2 to MOPr was examined using a BRET-based approach. AtT20 cells were plated in 10-cm dishes and co-transfected with MOPr C-terminally tagged with Rluc8 (MOPr-RLuc8), β-arrestin2-YFP and GRK2 (1 μg, 4 μg and 2 μg, respectively). 24 h after transfection wells were replated into white opaque 96-well plates (CulturPlate, PerkinElmer) and allowed to adhere overnight. Cells were washed with Hank's Balanced Salt Solution (HBSS) and equilibrated in HBSS for 30 min at 37° C. prior to the experiment. Coelenterazine h was added to a final concentration of 5 μM 10 min before dual fluorescence/luminescence measurement in a LUMIstar Omega plate reader (BMG LabTech). Baseline BRET was measured for 30 sec prior to addition of the indicated ligand. The BRET signal was calculated as the ratio of light emitted at 530 nm by YFP over the light emitted at 430 nm by Renilla luciferase 8 (RLuc8).

Assessment of MOPr Internalization

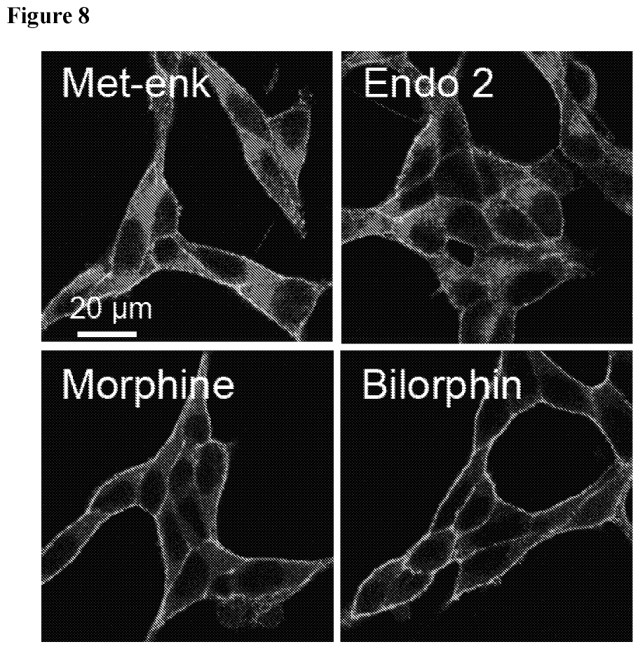

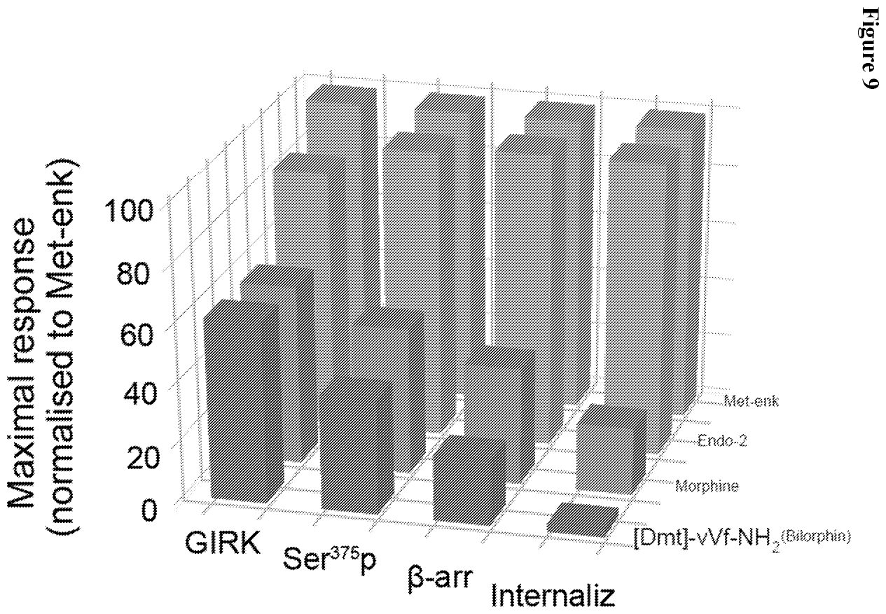

MOPr internalisation was assessed immunocytochemically after 30 minutes of agonist treatment (FIGS. 8, 9). Bilorphin produced almost no detectable internalisation of MOPr, compared to low level internalisation induced by morphine and robust internalisation driven by both endomorphin-2 and met-enkephalin (FIG. 9). Furthermore, co-incubation of bilorphin (10 μM) with an efficaciously internalizing agonist reduced internalisation (3 independent experiments using 3 μM DAMGO as agonist, data not shown). In summary, when normalised to the maximum response to met-enkephalin in each pathway, bilorphin displayed similar maximal G-protein efficacy to morphine with progressive reduction in relative efficacy across pathways from Ser³⁷¹ phosphorylation, β-arrestin recruitment to internalisation (FIG. 9), suggesting that bilorphin is a G-protein biased opioid.

Endocytosis assay: Receptor internalization was quantified using a ratiometric staining of membrane and internalized receptors. Briefly, AtT20 cells expressing FLAG-tagged MOPr were incubated with 1 μg/ml Alexa594-conjugated M1 monoclonal anti-FLAG (prepared from Alexa-fluor 594 with a succinimidyl ester moiety, Molecular Probes) for 30 min to label membrane receptors. Cells were then incubated for an additional 30 min with indicated agonist at 37° C. To unbind the M1 anti-FLAG antibody from the surface receptors, cells were quickly washed three times with ice-cold PBS lacking Mg²⁺ and Ca²⁺ and supplemented with 0.04% EDTA (pH 7.4). Cells were fixed with 4% paraformaldehyde in PBS for 20 min under non-permeabilized condition and then were incubated with anti-FLAG polyclonal antibody (1 μg/ml, 2 h at room temperature, Sigma Aldrich) followed by Alexa-fluor 488 goat anti-rabbit antibody (1 μg/ml, 1 h at room temperature, Thermo Fisher Scientific). Therefore, surface receptors were labeled with Alexa-fluor 488, while internalized receptors were labeled with Alexa-fluor 594. Percentage of internalized receptors was calculated as a ratio of mean 594 nm fluorescence intensity to total mean fluorescence intensity at 594 nm and 488 nm.

In experiments where GRK2-YFP and R-arrestin 2-HA were expressed, Alexa-fluor 405 goat anti-rabbit (2 μg/mL, 1 h at room temperature, Abcam) was used as a secondary antibody in place of Alexa-fluor 488 goat anti-rabbit to avoid fluorescence spectral overlap with YFP. 405 nm fluorescence was false-colored to green for representative images. Only YFP positive cells were analyzed for internalization.

### Example 8: A E_{MAX} and Δ log τ Determination

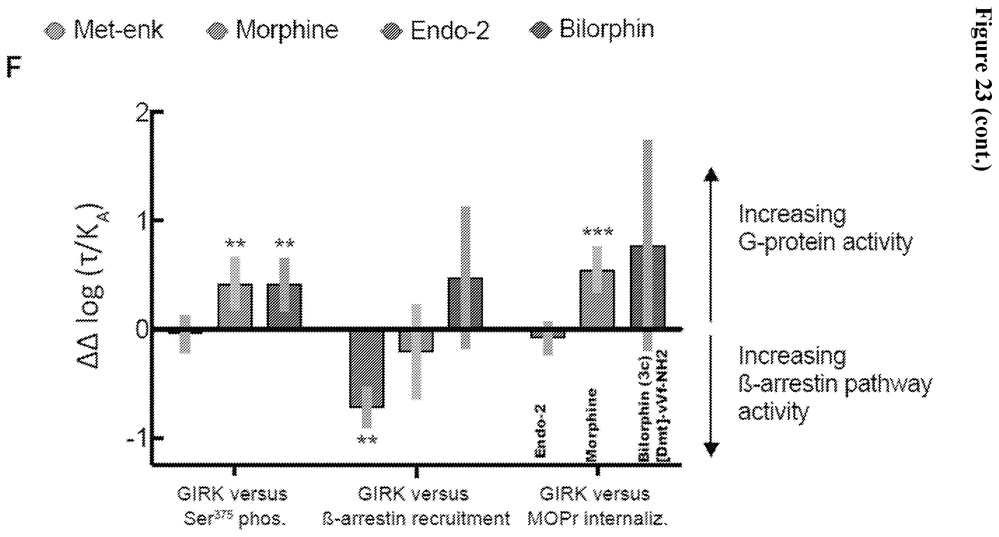

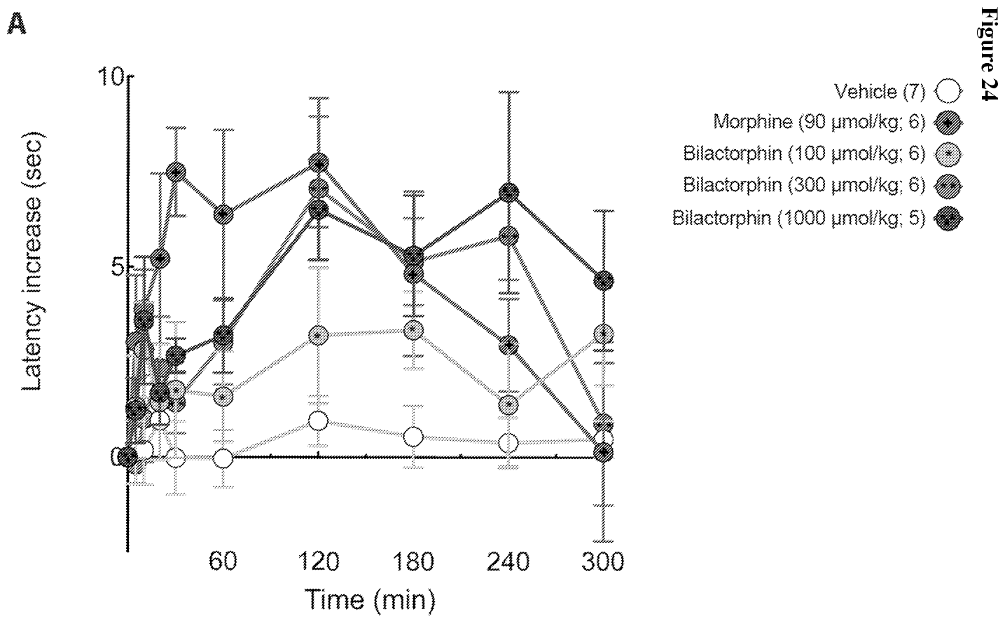

Operational analysis, the de facto standard for quantifying biased signaling (Kenakin Curr Protoc Pharmacol. (2016) 74:2.15.1-2.15.15; Kelly Br J Pharmacol (2013) 169(7): 1430-46), suggests that bilorphin is G-protein biased relative to morphine (FIG. 23) and relative bias values for other agonists, including the arrestin-biased endomorphin-2 were similar to those previously reported (Rivero et al Mol Pharmacol (2012) 82(2): 178-88). However, operational analysis requires accurate determination of EC₅₀, which was impractical for bilorphin due to very low internalisation efficacy, yielding poor functional affinity estimates with large error terms (FIG. 23) (Kelly Br J Pharmacol (2013) 169(7): 1430-46). Calculation of ΔE_{Max} ratios from maximum response in each pathway and, Δ log τ provide complementary estimate of bias in signalling assays where no ceiling exists, i.e. all agonists are partial agonists (Burgueño et al Sci Rep (2017) 7(1):15389; Kelly Br J Pharmacol (2013) 169(7): 1430-46). The current assay conditions, under which the maximum possible G_{GIRK} response for the high intrinsic efficacy agonist, met-enkephalin, was reduced to approximately 80% of the maximum possible response satisfies this criterion. This approach and accompanying data (FIGS. 18-22; Table 2) substantiated the G-protein bias of bilorphin relative to morphine, and to the strongly internalising peptides met-enkephalin (Thompson et al Mol Pharmacol (2015) 88(2): 335-346; McPherson et al (2010) Mol Pharmacol 78: 756-766) and endomorpin-2 (FIG. 23).

Bias calculation and statistics: Agonist concentration response curves were fitted to a three-parameter concentration response curve, a logistic function with constrained slope of 1, in GraphPad Prism 7 producing estimates of curve location (EC₅₀) and asymptote (Eₘₐₓ). As basal activity was subtracted in all pathways the bottom of the curve was constrained to 0.

The de facto standard for quantifying agonist affinity and efficacy to accurately determine biased signalling is the operational model of agonism (Black and Leff Proc R Soc Lond B Biol Sci (1983) 220(1219):141-62, Kenakin Mol Pharmacol (2015) 88(6):1055-61). Agonist concentration response data for each pathway was fitted to the operational model. Maximal effect in the system was defined by the reference full-agonist met-enkephalin and the slope of the transducer curve constrained to one. Efficacy (T) and affinity (KA) estimates were produced from the curve fit for test agonists endomorphin 2, bilorphin and morphine. log(τ/KA) values for each agonist were normalized by subtraction of the reference agonist met-enkephalin log(τ/KA) value within each pathway to produce Δ log(τ/KA). Subtraction across pathways produced ΔΔ log(τ/KA), a normalized estimated of each agonist's signaling bias. Previous papers on the operational model have advocated application of pooled variance in order to increase the power of these comparisons (Kenakin s al ACS Chem Neurosci (2012) 3(3):193-203). This approach is not suitable here, or in any situation with variable curve fit quality, due to the very low signaling efficacy of the biased agonists producing much larger error for the calculated parameters and invalidating the assumptions of pooled variance (Table 2). Standard error of the linear combinations of parameters was therefore propagated exactly under standard rules (Farrance and Frenkel Clin Biochem Rev (2012) 33(2):49-75, ISO/IEC Guide to Uncertainty in Measurement). Poor curve fit due to low signaling efficacy reduced the power of the ΔΔ log(τ/KA) approach generally and prevented confident interpretation of bias estimates from this approach (FIG. 23).

In cases where all test agonists are partial compared to the reference agonist efficacy alone has been used to quantify bias (Burgueño et al Sci Rep (2017) 7(1):15389). In systems with low receptor reserve the asymptote of the logistic function, Eₘₐₓ, is a robust, assumption free and affinity independent estimate of efficacy that approaches the value of operational efficacy. In systems with a linear relationship between agonist occupancy and effect such as the β-arrestin pathways studied here where there is no signal amplification, Eₘₐₓ approximates operational efficacy, ‘τ’.

Eₘₐₓ was normalized to the reference agonist within each pathway and subtracted across comparison pathways to produce Δ normalized Eₘₐₓ, an efficacy measure of bias. Observation of concentration-response curve position, which approaches operational affinity for all partial agonists presented here, across pathways measured shows bias in this instance does not appear affinity driven due to conservation of rank potency.

Degrees of freedom were calculated by first conservatively taking the lower of the two sample sizes when the normalizing to met-enkephalin. In the case of Δ normalized Eₘₐₓ, variance across the pathways could be assumed to be equal allowing degrees of freedom to be summed. In the case of ΔΔ log(τ/KA), low efficacy in the β-arrestin pathways caused heterogeneity of variance and degrees of freedom was approximated using the Welch-Satterwaite equation (ISO/IEC Guide to Uncertainty in Measurement).

Bias of each agonist, in both A normalized Eₘₐₓ and ΔΔ log(τ/KA) calculations, was tested by a one-way t-test to 0, the value of the reference agonist. The bias of bilorphin was then compared to morphine using a two-sample t-test, equal or unequal variance as appropriate. All comparisons were multiplicity adjusted using the Holm-Sidak ranking method (GraphPad Prism 7) within each pair of pathways examined.

TABLE 2
Pathway

| | | | | log(tau/ |
|---|---|---|---|---|
| Agonist | Eₘₐₓ | log(EC₅₀) | K_{A} | K_{A}) |
| | GIRK (80% knockdown) | | | |
| met-enkephalin | 0.84 ± 0.01 | −7.47 ± 0.03 | *** | 7.4 ± 0.03 |
| endomorphin-2 | 0.64 ± 0.01 | −6.9 ± 0.05 | −6.5 ± 0.06 | 7.5 ± 0.04 |
| morphine | 0.52 ± 0.02 | −7.6 ± 0.04 | −6.9 ± 0.07 | 6.7 ± 0.05 |
| bilorphin | 0.51 ± 0.01 | −7.2 ± 0.05 | −6.8 ± 0.06 | 7.0 ± 0.05 |
| oliceridine | 0.33 ± 0.01 | −7.6 ± 0.06 | −7.3 ± 0.09 | 7.2 ± 0.08 |
| | Phosphorylation | | | |
| met-enkephalin | 100* | −7.1 ± 0.06 | *** | 7.1 ± 0.05 |
| endomorphin-2 | 98 ± 2 | −7.2 ± 0.07 | *** | 7.2 ± 0.05 |
| morphine | 52 ± 3 | −6.4 ± 0.10 | −5.8 ± 0.12 | 5.9 ± 0.09 |
| bilorphin | 39 ± 4 | −6.1 ± 0.11 | −6.4 ± 0.12 | 6.2 ± 0.10 |
| oliceridine | Not tested | | | |
| | β-arrestin 2 recruitment | | | |
| met-enkephalin | 102 ± 4 | −6.3 ± 0.07 | *** | 6.2 ± 0.06 |
| endomorphin-2 | 100 ± 3 | −7.0 ± 0.06 | *** | 7.0 ± 0.06 |
| morphine | 41 ± 4 | −6.1 ± 0.20 | −5.9 ± 0.24 | 5.7 ± 0.19 |
| bilorphin | 18 ± 4 | −6.1 ± 0.34 | −6.0 ± 0.39 | 5.3 ± 0.32 |
| oliceridine | Not tested | | | |
| | Internalisation | | | |
| met-enkephalin | 100* | −6.1 ± 0.05 | *** | 6.1 ± 0.03 |
| endomorphin-2 | 94 ± 3 | −6.4 ± 0.07 | *** | 6.2 ± 0.05 |
| morphine | 23 ± 2 | −5.4 ± 0.12 | −5.4 ± 0.21 | 4.8 ± 0.08 |
| bilorphin | 3 ± 1 | −5.1 ± 0.40 | −6.6 ± 2.4 | 4.9 ± 0.47 |
| oliceridine | No activity | | | |

*Defined as 100%
***Full agonist, constrained K_{A}

### Example 9: MOPr Internalisation in Cells Overexpressing GRK-YFP

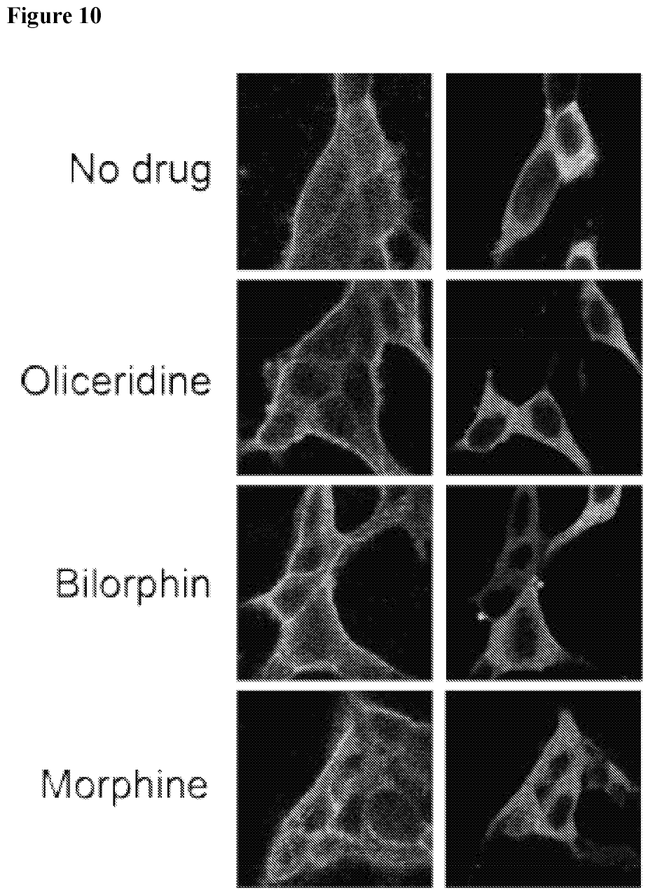

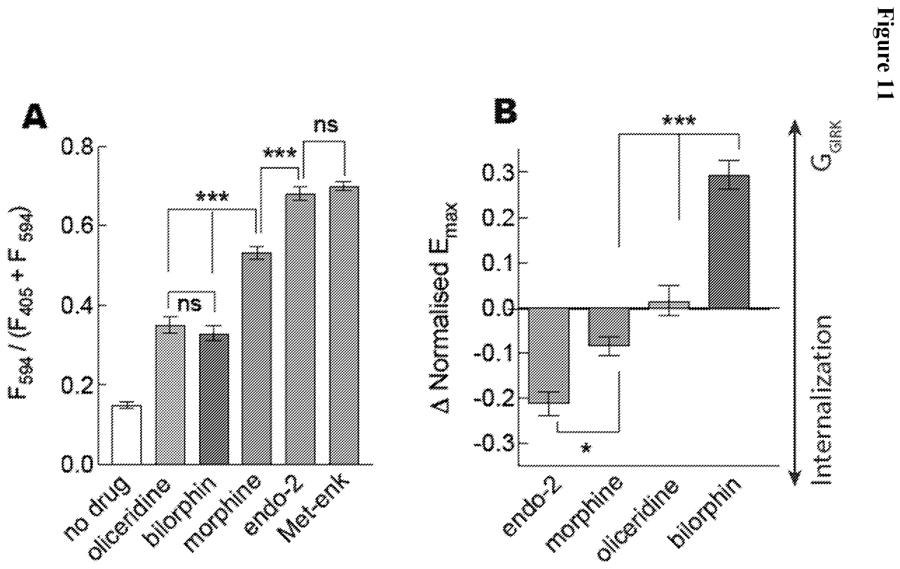

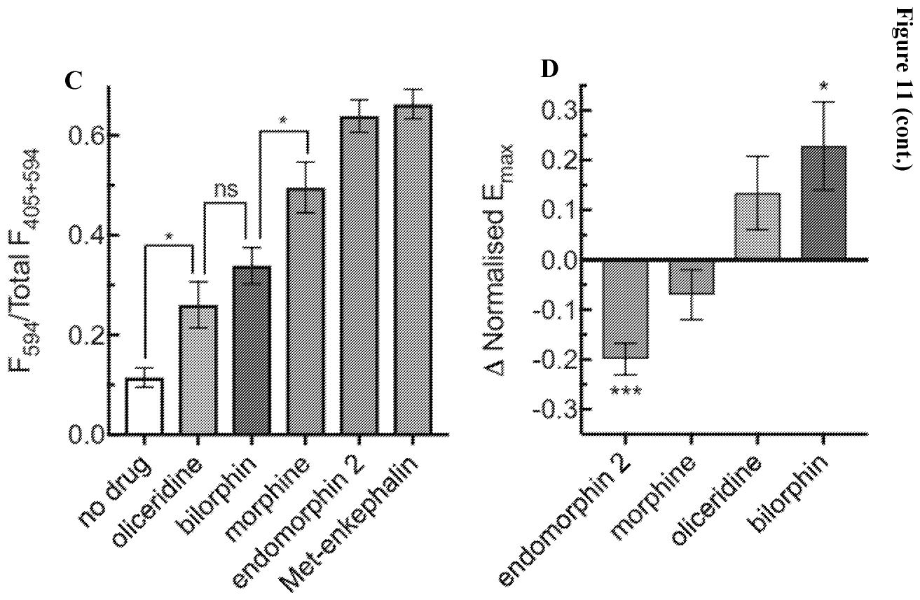

To compare the bias of bilorphin with the established, small non-peptidic, G-protein biased agonist, oliceridine, both of which produce very little internalisation, MOPr internalisation in cells overexpressing GRK2-YFP and B-arrestin 2-HA was examined, which was undertaken to enhance internalisation of morphine (Zhang et al Proc Natl Acad Sci USA (1998) 95(12):7157-62). In GRK2 positive cells, morphine, oliceridine and bilorphin all produced clear internalisation signals (FIG. 10). Quantification shows that even under these amplified conditions, bilorphin induces similar MOPr internalisation to oliceridine but less than morphine at saturating concentrations (FIGS. 10, 11A, 11B). Calculation of bias relative to morphine with the ΔEₘₐₓ method suggests that bilorphin exhibits similar or greater G-protein bias than oliceridine, establishing it is a novel, and also potentially safer G-protein biased opioid (FIG. 11B). See also FIGS. 11C and 11D.

### Example 10: Molecular Docking and Molecular Dynamics

To investigate whether there is a conformational basis for bias, molecular docking and molecular dynamics (MD) simulations (Sutcliffe et al J Mol Biol (2017) 429(12):1840-1851) were performed with bilorphin and endomorphin-2 at mMOPr. Conformations (10,000) were taken from one microsecond simulations of each peptide in water. These ensembles were docked to the orthosteric binding pocket of the inactive MOPr crystal structure (Manglik et al Nature (2016) 537(7619): 185-190) using the Bristol University Docking Engine (BUDE) (McIntosh-Smith et al Int J High Perform Comput Appl (2015) 29(2):119-134). The lowest energy structures were inspected visually and prioritised according to the distance between the protonated amine of the ligand and Asp147³³² (superscript numbers follow the Ballesteros-Weinstein numbering system for GPCR residues [Ballesteros and Weinstein Methods in Neurosciences (1995) 25:366-428] of less than 3 Å. Selected peptide-MOPr complexes were then embedded in a lipid and cholesterol bilayer and used in all-atom MD simulations to evaluate binding poses, residue interactions and receptor conformational changes. 8×125 ns simulations were performed, with different initial velocities, to give a total of 1 μs of trajectory data for each peptide. Endomorphin-2 was modelled as the cis isomer, as this conformation had the most stable and lowest energy binding pose after docking with BUDE and 125 ns of MD simulation.

The predicted binding pose of bilorphin determined from the MD simulations is shown in FIGS. 18A and 18B. Bilorphin was predicted to bind within the orthosteric binding site, with the Dmt-Tyr1 orientated towards the intracellular side of MOPr. The rest of the tetrapeptide chain extended out towards the extracellular side of MOPr, making contacts with residues at the top of TM2 and TM7. Endomorphin-2 was also predicted to bind in the orthosteric site (FIGS. 18C, 18D), with the phenol group of Tyr1 interacting with His297^{6.52}, and the rest of the peptide chain extending towards ECL1 and ECL2 and the top of TM2. The RMSD plot in FIG. 19A shows that the binding pose of bilorphin was relatively stable over the 1 μs simulation time after an initial deviation from the docked pose of ˜1.5 Å. The backbone of endomorphin-2 was stable in its bound pose, with some fluctuation in the RMSD plot introduced by the flexibility of the Phe4 aromatic ring which switched between 3 main poses (FIG. 19B).

Both peptides maintained an ionic interaction with the essential opioid binding residue Asp147^{3.32} of the MOPr complex, and interacted with the conserved rotamer toggle switch Trp293^{6.48}, for the entire simulation time (FIG. 20A). Both peptides also interacted with His297^{6.52}; endomorphin-2 directly, and bilorphin switching between a direct interaction and hydrogen bonding via a bridging water molecule (inset in FIG. 18A). However, there were differences in how these peptides interact with the MOPr binding pocket. For instance, bilorphin, but not endomorphin-2, interacted with Tyr75^{1.39} in TM1. On the other hand, endomorphin-2 interacted with the extracellular loops, contacting W133^{ECL1} in ECL1 for the entire simulation time, and transiently interacting with Cys217^{ECL2}, Thr218^{ECL2} and Leu219^{ECL2} of ECL2, but these interactions were absent for bilorphin. Moreover, endomorphin-2 made a greater number of contacts with TM3 and TM5 than bilorphin.

Principal component analysis (PCA) was employed to examine the conformational changes in the receptor transmembrane helices. After fitting to remove global rotation and translation of the system, the covariance matrix was generated from just the alpha carbons of the MOPr transmembrane domains, to avoid including the highly dynamic loops in the analysis. The receptor conformations at each time point were projected onto principal components (PC) 1 and 2, and plotted in FIG. 20B. PC1 and PC2 accounted for 28.9% and 10.9% of the variance, respectively. Both peptide-MOPr complexes sampled conformations across PC2, but clustered differently based on PC1. By generating a pseudo-trajectory of PC1, and extracting structures from the actual simulations that represent extremes of PC1, we were able to visualise the helix movements contributing to the principal component.

As depicted in FIG. 21, PC1 primarily described alternative conformations in the extracellular region of the receptor close to the orthosteric binding site, and to a lesser extent differences in the intracellular portions of the helices. With bilorphin bound, there was a bulging of the middle portion of TM1 and a shift outwards from the helix bundle, relative to the endomorphin-2-bound receptor. There were also substantial movements of the extracellular ends of TMs 2, 6 and 7, and a kink formed in TM4 allowed the extracellular part of this helix to move towards TM3 with endomorphin-2 bound. A smaller movement of TM3 towards TM2 in the endomorphin-2-bound receptor, whereby Met151^{3.36} shifted ˜1.7 Å from its initial position, is in agreement with the active conformation of TM3 observed in the agonist-bound crystal structure (Huang et al Nature (2015) 524(7565): 315-21). These alternative conformations of the helices around the orthosteric binding site were also reflected in the volume of the binding pocket, as calculation of the volume of the orthosteric binding site using CASTp revealed the bilorphin binding pocket volume to be on average 1.6 times greater than with endomorphin-2 bound (Dundas et al Nucleic Acids Res (2006) 34(Web Server issue):W116-8) (FIG. 22).

On the intracellular side of MOPr, PC1 described inward movements of TMs 5, 6 and 7 with endomorphin-2 bound, compared to the bilorphin-bound MOPr (FIG. 21).

The cryo-electron microscopy structure of the MOPr-Gi complex bound to DAMGO was recently resolved (Koehl Nature (2018) 558: 547-552). Prior to the release of this structure, the inventors performed MD simulations with DAMGO at the MOPr (FIG. 28) using the methods described here for bilorphin and endomorphin-2. The position of DAMGO as well as the ligand residue interactions in the cryo-EM structure and in the model (FIG. 28) were nearly identical, providing confidence that our docking and MD strategy for bilorphin and endomorphin-2 is likely to be relevant to the ligand-MOPr interactions occurring in vivo.

Analysis of the MD data therefore suggests that the different ligand-residue interactions for these oppositely biased peptides may lead to the alternative receptor conformations described by the PCA, and hence the opposing bias profiles of bilorphin and endomorphin-2.

Molecular dynamics: Generation of peptide conformations: Generation of peptide conformations: The 3D conformers of bilorphin and endomorphin-2 (EM2) were built in Chimera (Petterson et al J Comput Chem (2004) 25(13): 1605-12). Two endomorphin-2 conformers were used, with the Tyr1-Pro2 peptide bond modelled as either the cis or trans isomer, and treated as separate ligands for MD simulation and docking. Peptides were protonated at the N-terminal tyrosine and parameterised with Antechamber and the general Amber force field (Wang et al J Mol Graph Model (2006) 25(2): 247-60, Wang et al J Comput Chem (2004) 25(9): 1157-74). Conformer generation was performed by running 1 μs MD simulations of each peptide in explicit solvent (0.15 M NaCl and TIP3P water) under the Amber ff14SB force field. These trajectory data were analysed with cpptraj (Roe et al J Chem Theory Comput (2013) 9(7): 3084-95) to extract 10000 conformations for each peptide to use in molecular docking.

Docking of peptides to MOPr: Molecular docking was performed with the Bristol University Docking Engine (BUDE) (McIntosh-Smith et al Int J High Perform Comput Appl. (2015) 29(2): 119-134.). Peptides were docked to an inactive MOPr model obtained from the x-ray crystal structure of the antagonist-bound MOPr (PDB: 4DKL) (Manglik et al Nature (2012) 485(7398): 321-6). The protein was prepared in Insight II (Accelrys) as follows; ligands and the T4 lysozyme were removed, and a loop search performed to find a homologous loop to model in the missing intracellular loop 3. A loop was selected by visual inspection and the residues changed to the correct mouse MOPr sequence. Molecular docking to this MOPr structure was performed with each of the three peptides, bilorphin, cis-endomorphin-2 and trans-endomorphin-2, independently. The following describes the docking procedure for one peptide. Multi-conformer docking was run such that the 10000 conformations of the peptide were treated as independent molecules. A box of size 15, 15, 15 Å centred on the orthosteric binding site was designated as the search space. BUDE's genetic algorithm was used to search the available pose space for the best energy poses. A total of 105,000 poses were sampled for each of the 10,000 peptide conformers. The total possible number of poses was 1.57×10⁸ for each conformer, corresponding to x,y,z translation within the box and 360° rotation in all axes in 10° increments. The 50 lowest energy binding poses were inspected visually and subjected to a distance constraint between the protonated amine of the ligand and Asp147^{3.32} of less than 3 Å. The selected peptide-MOPr complexes were used in short (125 ns) MD simulations to assess the stability of the binding pose, before a full 1 μs of trajectory data was collected, as described below. Based on the docking data and the initial 125 ns MD simulations, the cis-endomorphin-2 conformer was chosen for further simulation.

MD simulations: Each peptide-MOPr complex was embedded in a POPC:POPE:cholesterol lipid bilayer at a 5:5:1 ratio using the replacement method, and the simulation box (initial dimensions: 90, 110, 90 Å) solvated with TIP3P water and NaCl (150 mM), using the CHARMM-GUI software (Jo et al J Comput Chem (2008) 29(11): 1859-65). Amber parameter topology and coordinate files were prepared in LEaP. Structures were minimised over 10000 steps, then the system was heated under constant volume and pressure with lipids restrained, from 0 K to 100 K over 5 μs, and then from 100 K to 310 K over 100 μs. 10 rounds of 500 μs equilibration was performed under constant pressure to equilibrate the periodic box dimensions. Simulations were run in 8×125 ns parallel steps under the Amber ff14SB and Lipid14 force fields (Maier et al J Chem Theory Comput (2015) 11(8): 3696-713; Dickson et al J Chem Theory Comput (2014) 10(2): 865-79), producing 1 μs of simulation data for each peptide-MOPr complex. Temperature and pressure were controlled using the Langevin thermostat and the anisotropic Berendsen barostat, with a 2 fs time step and trajectories written every 100 μs. Trajectories were visualised in VMD (Humphrey et al J Mol Graph (1996) 14(1): 33-8, 27-8), analysis was performed using cpptraj (Roe et al J Chem Theory Comput (2013) 9(7): 3084-95), and images were prepared in Chimera (Petterson et al J Comput Chem (2004) 25(13): 1605-12).

Principal Component Analysis: Trajectories were aligned to a set of “core residues” showing the least fluctuation during the simulation time to remove general translation and rotation of the protein in analysis. Principal component analysis was performed on the 3D Cartesian coordinates of the alpha carbons of the transmembrane domains of all trajectories, yielding 567 eigenvalues. Receptor conformations at each simulation time point were projected onto the first 2 PCs, accounting for ˜40% of the variance.

### Example 11: In Vivo Assessment

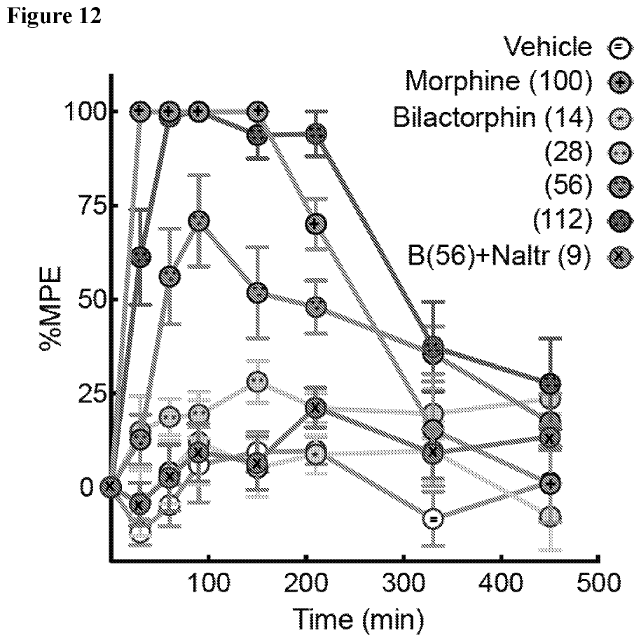

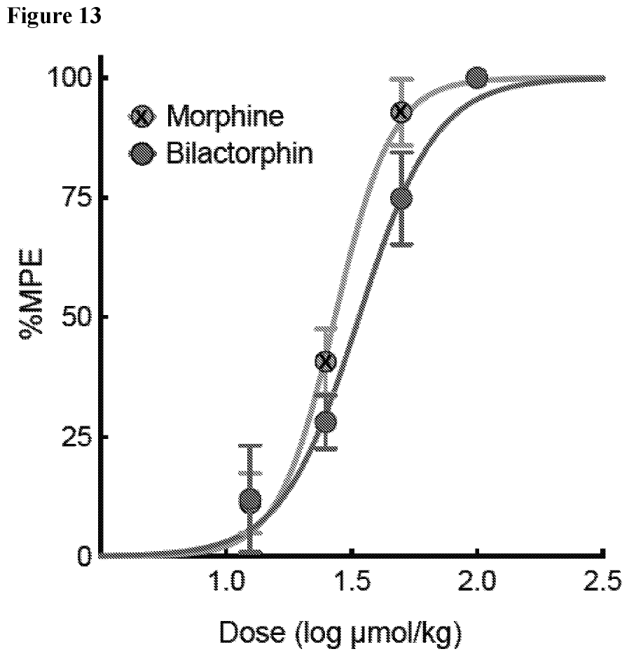

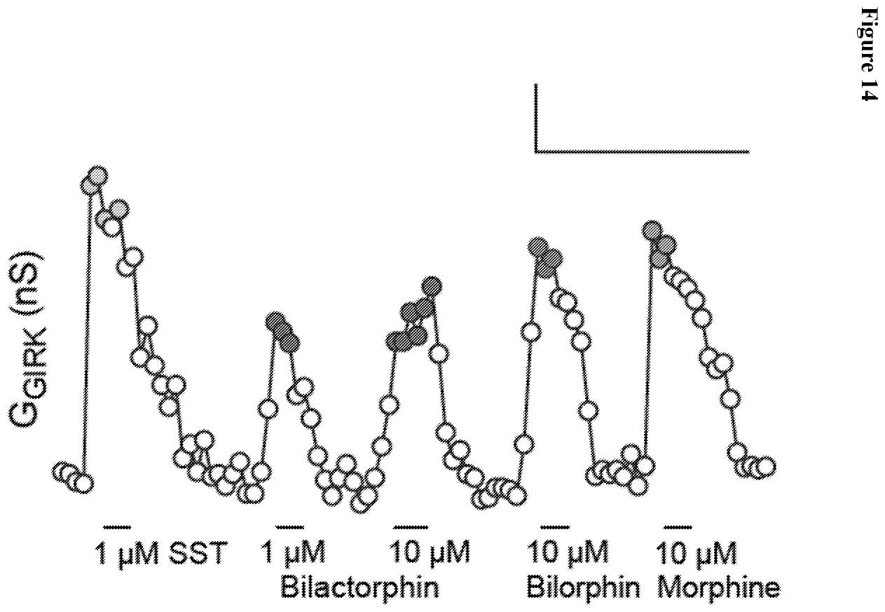

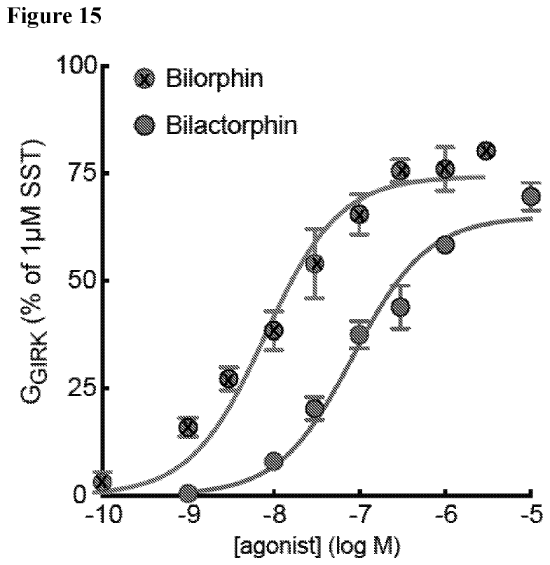

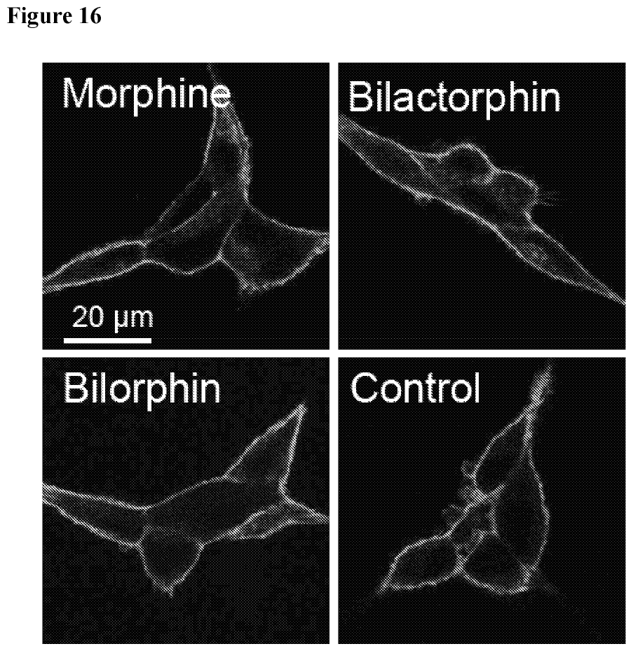

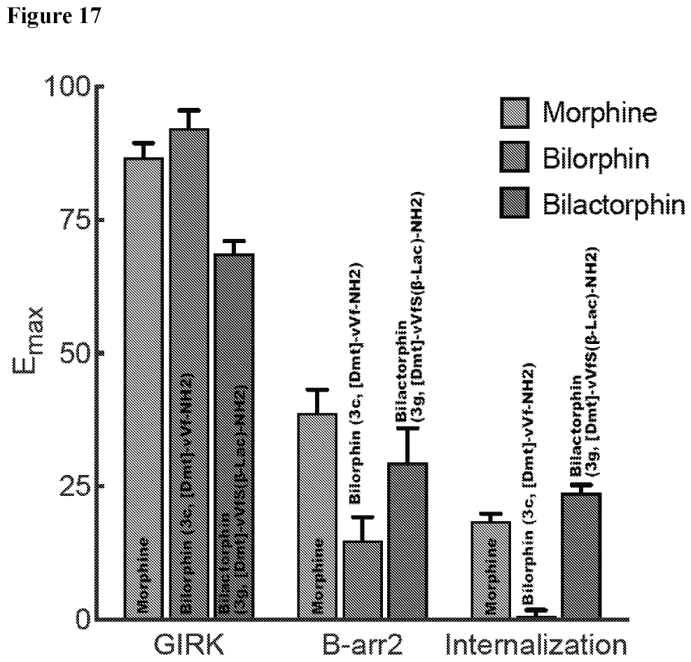

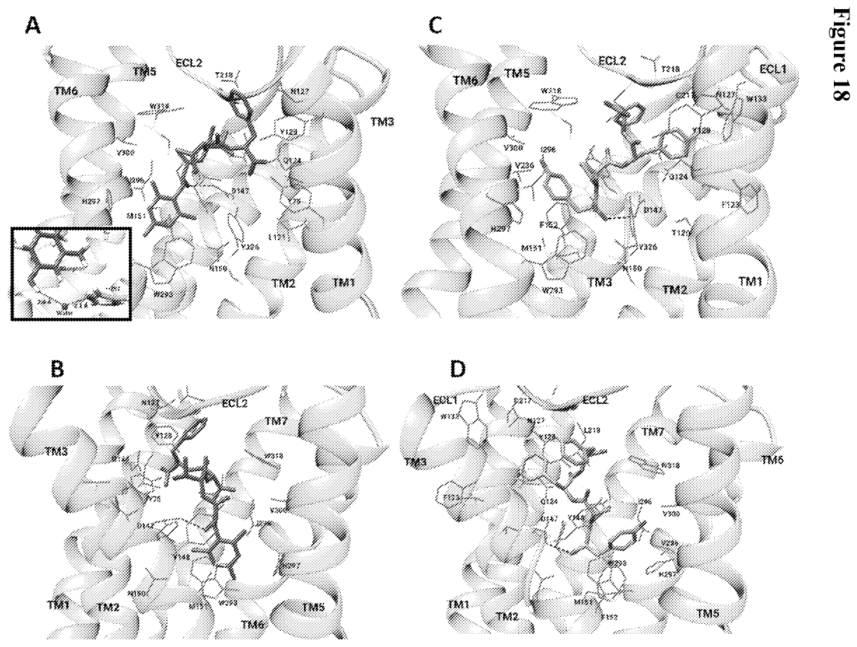

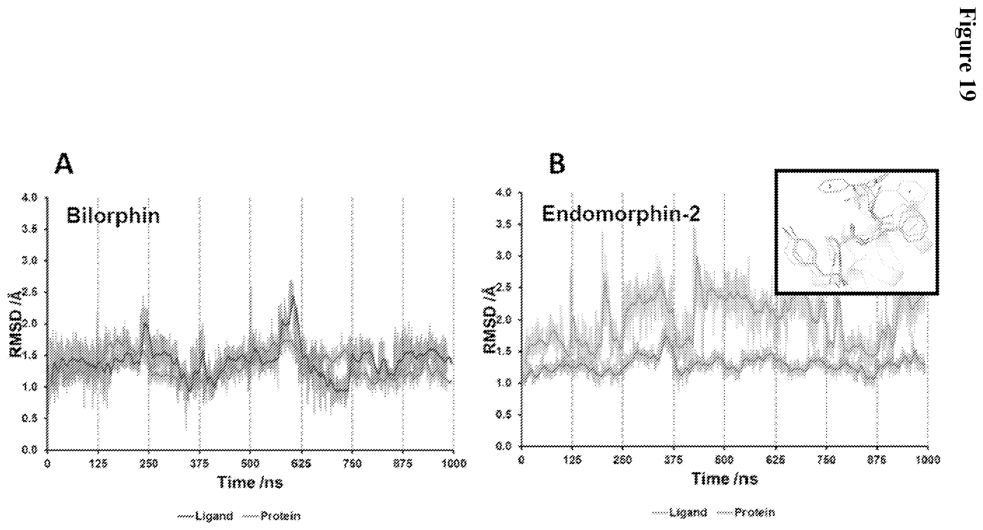

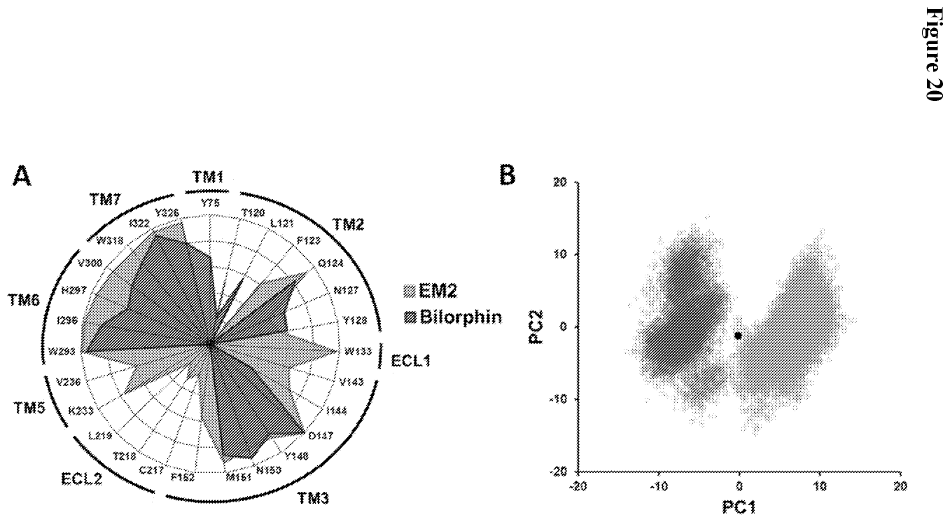

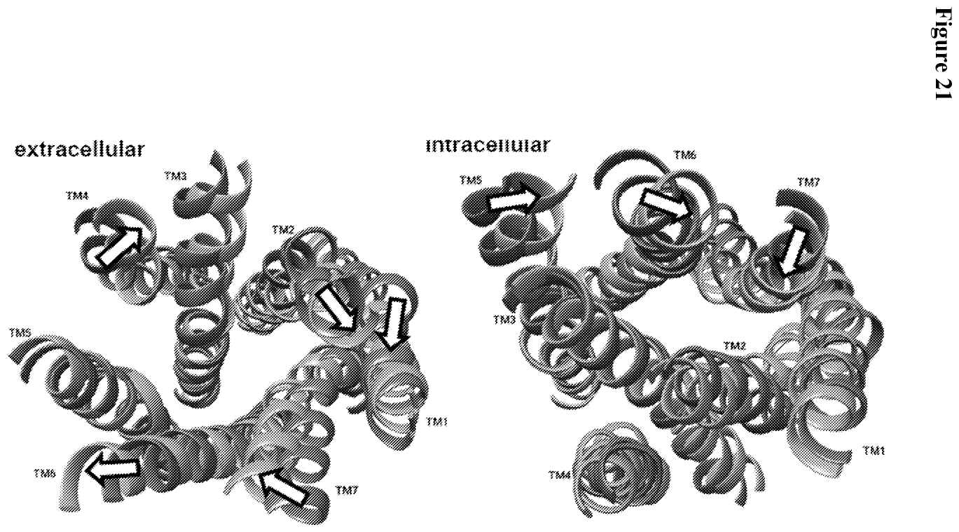

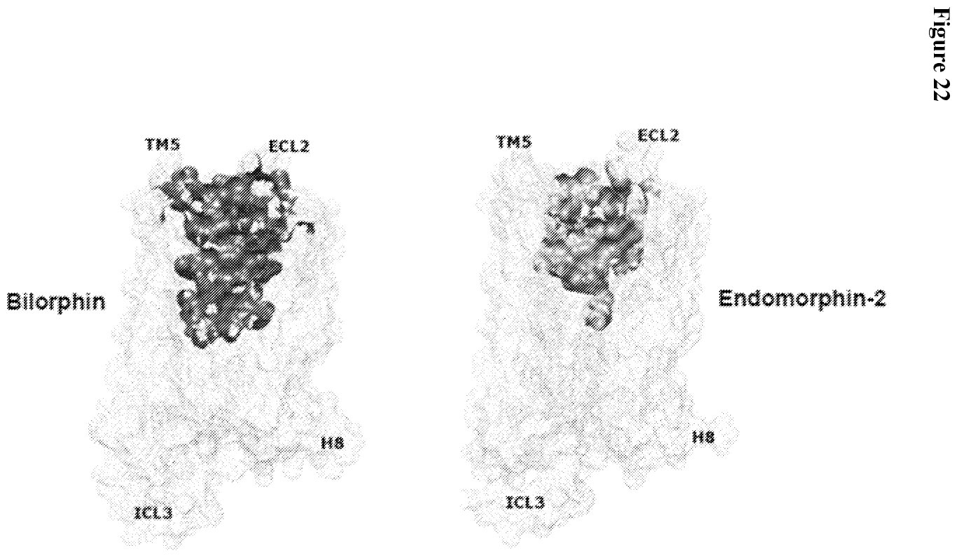

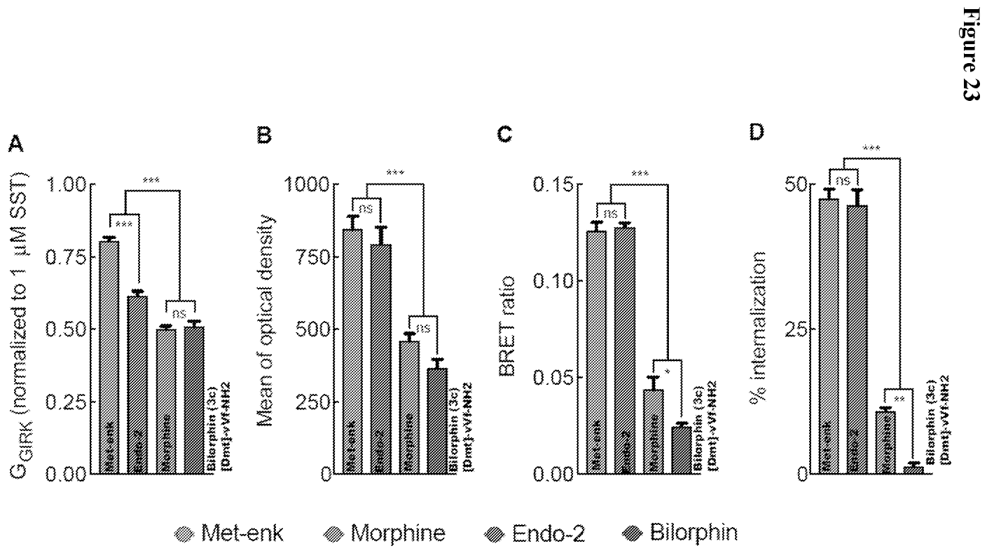

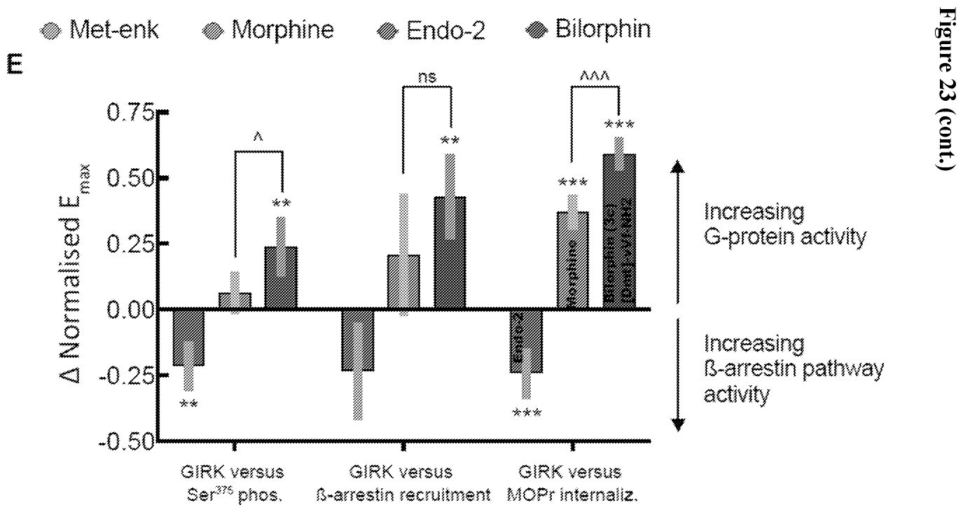

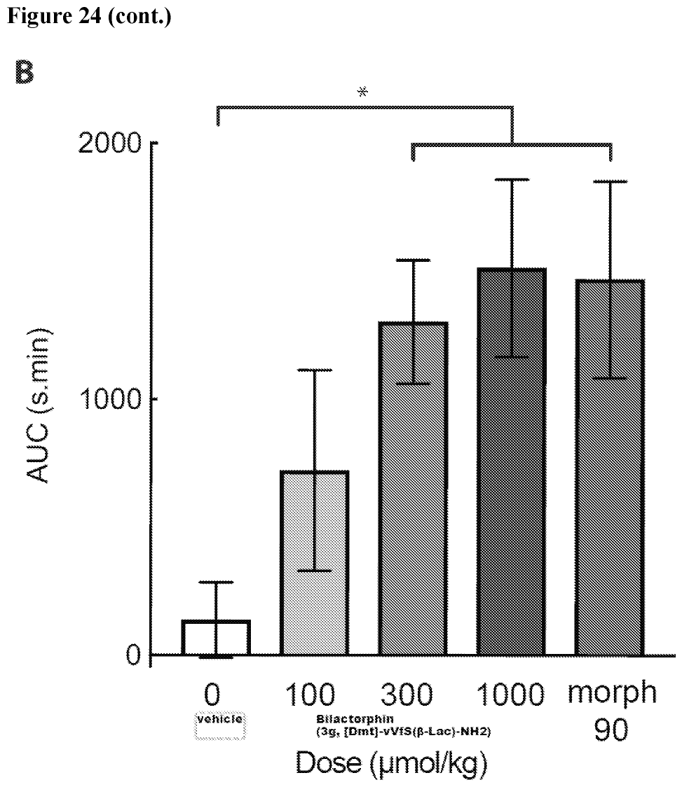

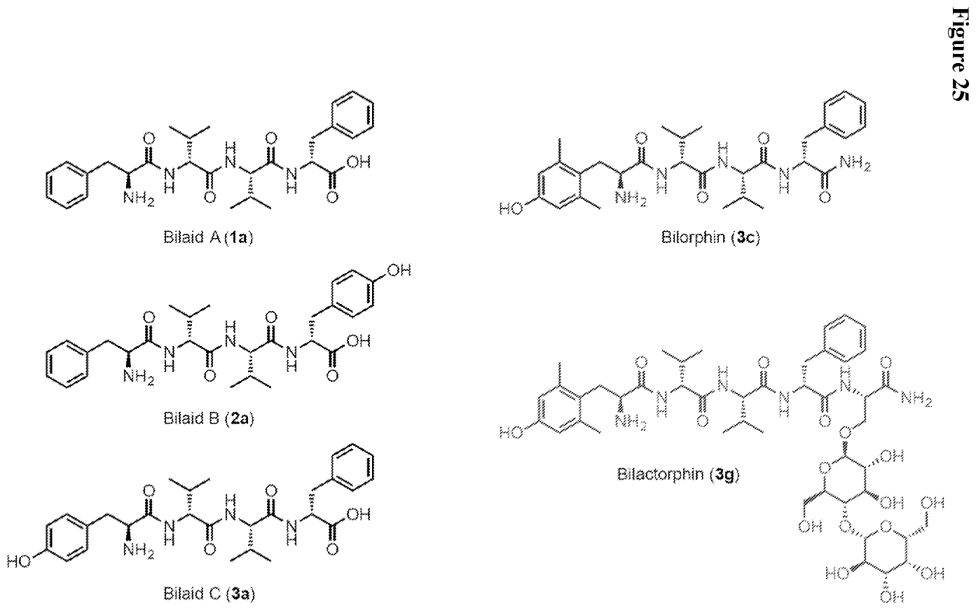

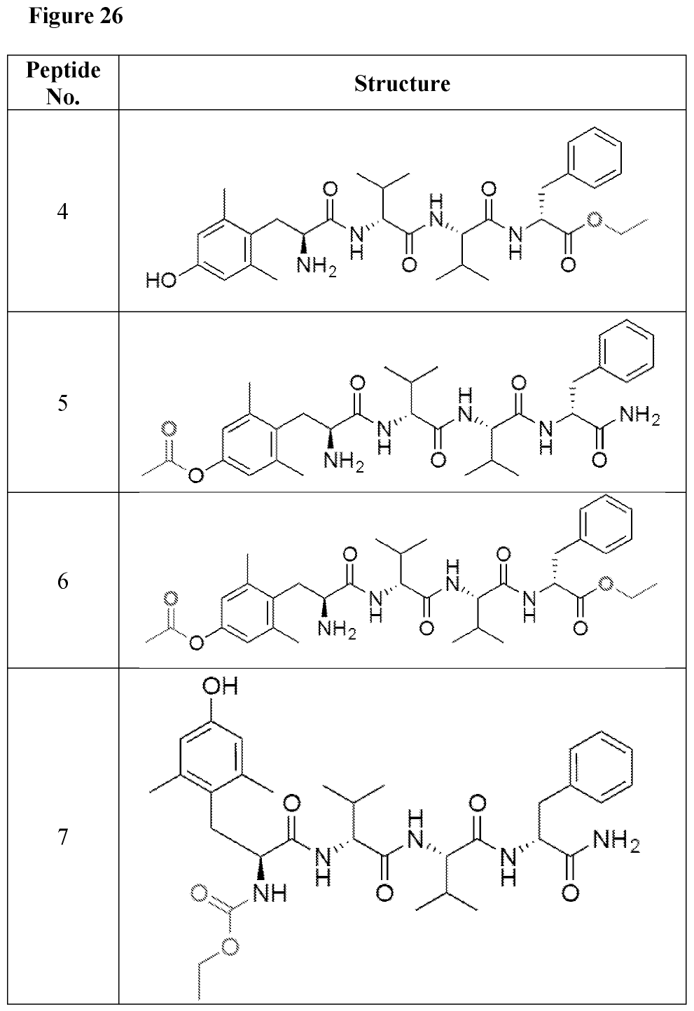

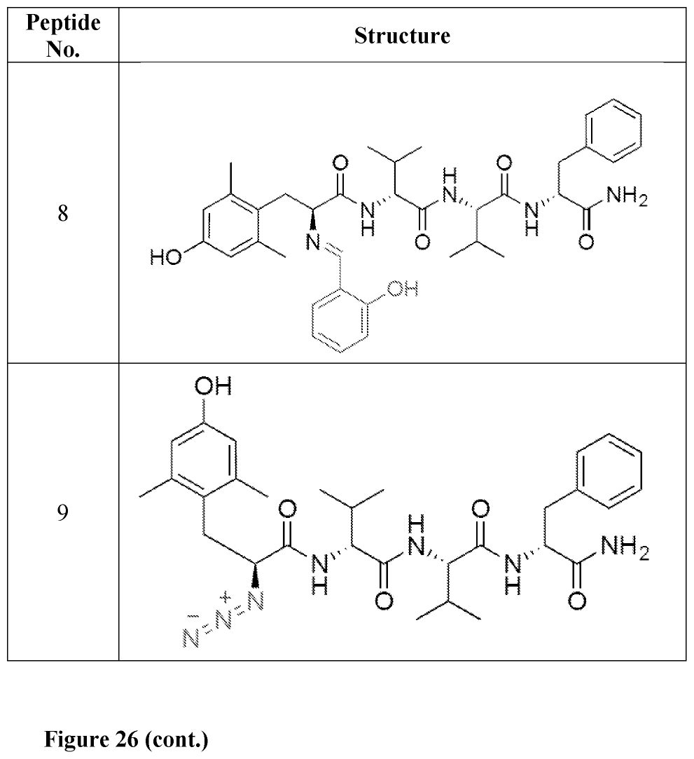

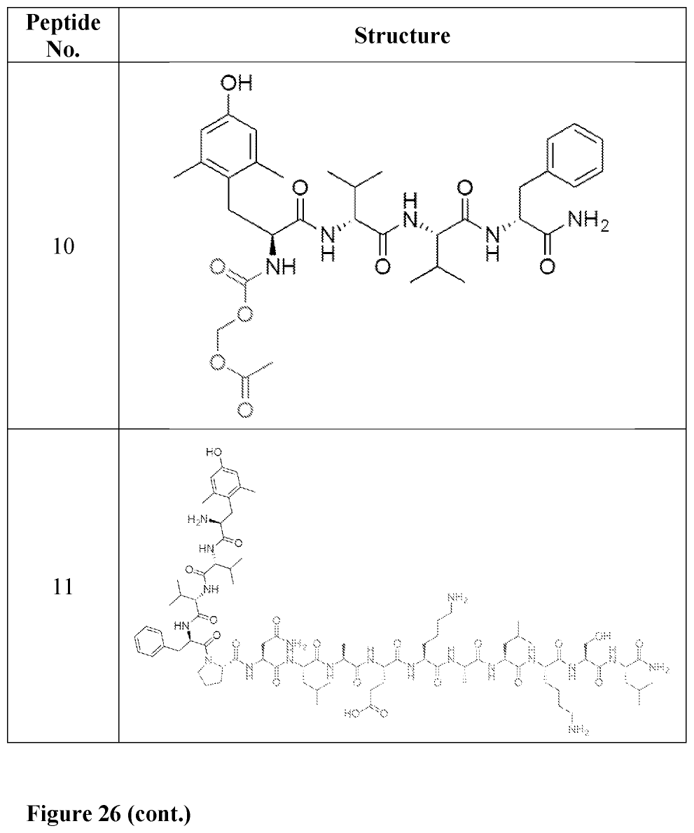

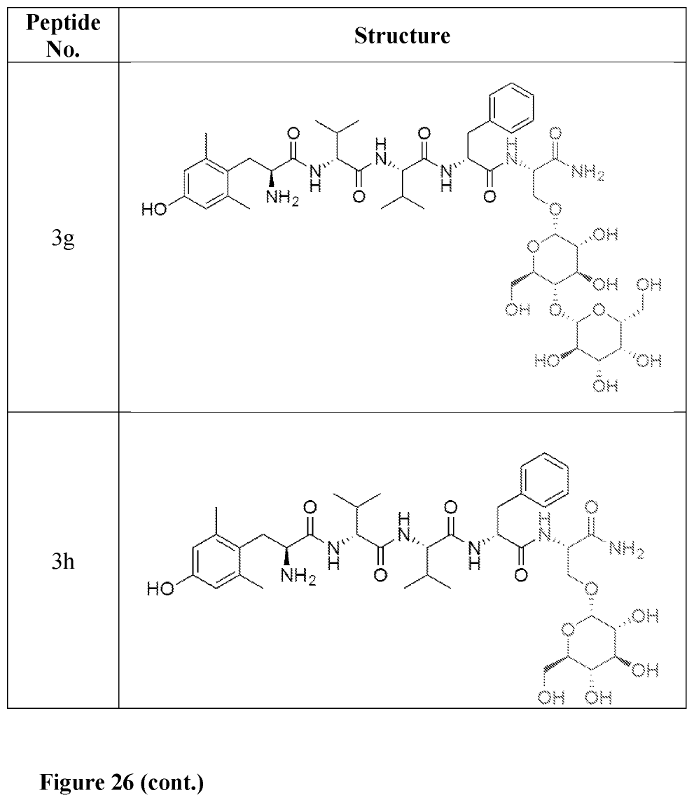

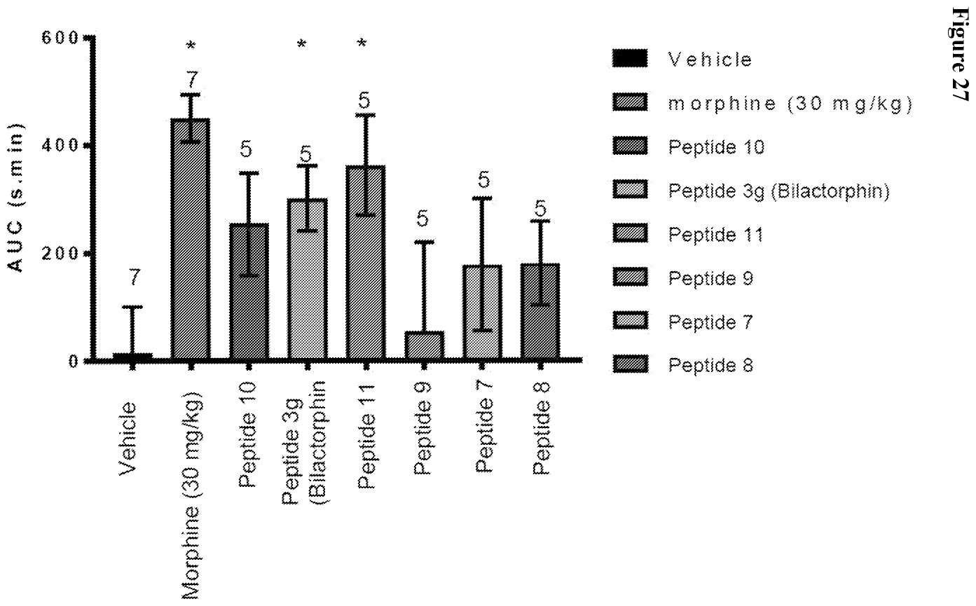

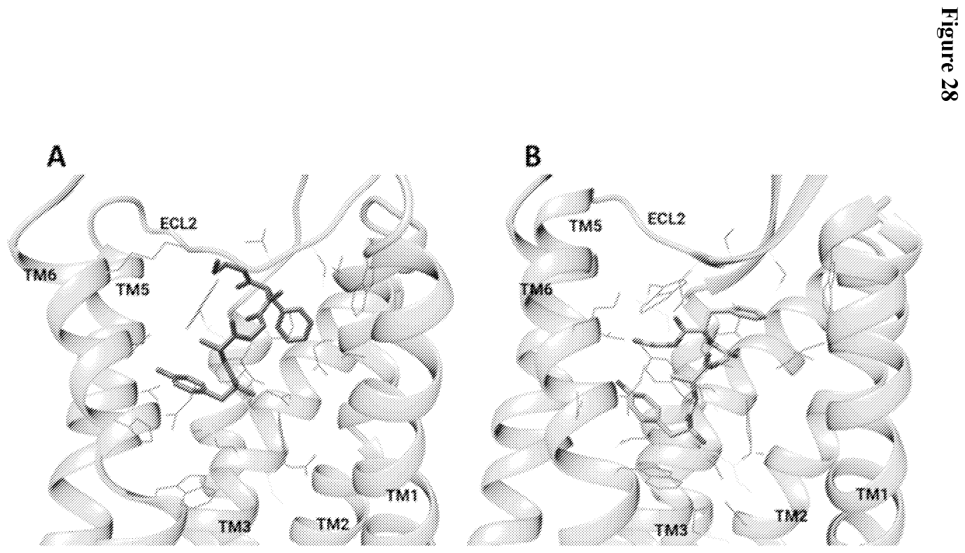

The actions of bilorphin were assessed in vivo. Bilorphin failed to inhibit nociception in the hotplate test when administered subcutaneously (100 mg/kg, n=4) or intravenously (50 mg/kg n=4) versus vehicle. By contrast, bilorphin was antinociceptive after intrathecal injection (5 nmol/mouse, peak effect 41±9% MPE n=4, versus 0±1.5% for vehicle, n=4), suggesting the lack of systemic activity is due to poor penetration of the blood brain barrier (BBB). Several bilorphin analogues were developed with substitutions thought to enhance BBB permeability, including gycosylation near the C-terminal. The di-glycosylated analogue, bilactorphin (3g), was a potent analgesic after systemic administration (s.c; ED₅₀ of 34 μmol/kg, 95% CI=28-40 μmol/kg; FIGS. 12, 13) and was nearly equipotent with morphine (ED₅₀ of 27 μmol/kg, 95% CI=24-30 μmol/kg; FIG. 13) and was antagonised by co-administration of the opiod antagonist, naltrexone (FIG. 12). Bilactorphin was also active after i.v (peak effect of 88.9±11.8 versus 14.4±1.8% MPE for vehicle, n=3-4) or oral administration (FIGS. 24A, 24B) In contrast the mono-glycosylated analogue (3h) was systemically inactive, consistent with the greater analgesic effects elicited by systemic administration of disaccharide vs. monosaccharide modified opioid peptide. (Li et al Future Med Chem (2012) 4(2): 205-26). These findings establish that the LDLD opioid peptide backbone is a viable framework for further development of G-protein biased opioid analgesics. Like bilorphin, bilactorphin, was a potent partial opioid agonist in AtT20 cells (without fractional inactivation of MOPr) but exhibited a small loss of potency compared with bilorphin (FIGS. 14, 15). Bilactorphin did, however, display modest internalization and β-arrestin recruitment compared to bilorphin suggesting the potential superiority of other substitutions that do not disrupt G-protein bias of the parent bilorphin (FIGS. 16, 17).

Nociception (Analgesia) testing: All experiments involving animals were approved by the University of Sydney Animal Ethics Committee (AEC. Protocol number K00/12-2011/3/5650). Experiments were performed under the guidelines of the Australian code of practice for the care and use of animals for scientific purposes (National Health and Medical Research Council, Australia, 7th Edition). Great care was taken to minimise animal suffering during these experiments and to reduce the number of animals used. Adult male C57BL/6 mice (20-25 g) were housed 5-6 per cage in individually ventilated cages under controlled light (12:12 h, lights on at 6 am) and climate (18-23° C., 40-60% humidity) conditions. Food and water was available ad libitum. Mice were given at least 7 days to habituate to housing facilities prior to handling, and handled by the experimenter for 4 days prior to testing. Experiments were conducted between 8 am and 6 μm in a quiet, temperature-controlled room (21±1° C.). The experimenter was blind to all drugs tested. Animals were tested on a 54° C. hotplate, with a maximum cut-off time of 20 seconds to prevent tissue damage. Endpoints were hindpaw lick, hindpaw flutter or jump. Baseline latency was recorded immediately before subcutaneous injection with morphine, bilactorphin or vehicle (20% PEG400/saline v/v) in a total volume of 200 μL. Mice were tested 30, 60, 90, 150, 210, 330 and 450 min following injection. The percentage of maximal possible effects (% MPE) were calculated as follows: % MPE=(test latency−baseline latency)/(cutoff latency−baseline latency)×100%. The cut-off latency was 20 seconds. Significant differences were assessed with a two-way ANOVA and Tukey's post hoc multiple comparisons test. The dose-response curves for bilactorphin and morphine were calculated using the maximal responses for each dose between 30-90 min. Doses were transformed to the logarithm of μmol/kg. A two-way ANOVA was used to compare data at equimolar doses.

### Example 12: In Vivo Assessment

The peptides of FIG. 27 were tested exactly as described above in Example 11. Experiments were performed under the guidelines of the Australian code of practice for the care and use of animals for scientific purposes (National Health and Medical Research Council, Australia, 7th Edition). Great care was taken to minimise animal suffering during these experiments and to reduce the number of animals used. Adult male C57BL/6 mice (20-25 g) were housed 5-6 per cage in individually ventilated cages under controlled light (12:12 h, lights on at 6 am) and climate (18-23° C., 40-60% humidity) conditions. Food and water was available ad libitum. Mice were given at least 7 days to habituate to housing facilities prior to handling, and handled by the experimenter for 4 days prior to testing. Experiments were conducted between 8 am and 6 μm in a quiet, temperature-controlled room (21±1° C.). The experimenter was blind to all drugs tested. Animals were tested on a 54° C. hotplate, with a maximum cut-off time of 20 seconds to prevent tissue damage. Endpoints were hindpaw lick, hindpaw flutter or jump. Baseline latency was recorded immediately before subcutaneous injection with morphine, peptide, or vehicle (20% PEG400/saline v/v) in a total volume of 200 μL. Mice were tested 30, 60, 90, 150, 210, 330 and 450 min following injection. The percentage of maximal possible effects (% MPE) were calculated as follows: % MPE=(test latency−baseline latency)/(cutoff latency−baseline latency)×100%. The cut-off latency was 20 seconds. Integrated Area Under the Curve over one hour (AUC: response in seconds×time in minutes) for hotplate responses measured, 5, 10, 20, 30 and 60 minutes after subcutaneous injection of each drug or saline were calculated for each drug by measuring triangulated areas of response (seconds) multiplied by time of testing (minutes) for 60 minutes at the times specified. Differences were analysed with a one way ANOVA with Fisher's LSD post-hoc tests.

### Discussion

The invention relates to the identification of a novel peptidic backbone useful for the development of a new peptidic class of G-protein biased opioids. The results generated indicate that his peptidic backbone can be used to develop an orally active opioid agonist with G-protein biased pharmacology. This novel LDLD structure has not previously been isolated from a eukaryote. The parent natural product, bilaid C (3a in Table 1) from which bilorphin was derived, is a relatively weak opioid and the potential natural function of the opioid agonist activity for this estuarine yeast is uncertain. The unexpected biostability of the LDLD structure and its novel opioid pharmacology, can be used to develop safer opioids.

G-protein biased opioid agonists have been proposed as a route to improving therapeutic profile. Among known peptidergic opioid agonists, which have little bias or bias toward β-arrestin signalling, bilorphin's pharmacological profile is most unusual as it is atypically G-protein biased compared with other natural opioid peptides, although a synthetic opioid cyclopeptide with G-protein bias was recently reported. Bilorphin is comparably biased to the Phase III drug candidate oliceridine. Glycosylation produced an analogue active in vivo via subcutaneous and oral administration, validating the bilorphin tetrapeptide backbone as a platform for further development of biased opioid agonists. Pre-clinical development of such G-protein biased agonists show a strikingly favourable profile with reduced respiratory depression and constipation. The first such compound to reach clinical trials, oliceridine (TRV130), was reported to have an increased window between antinociceptive and respiratory depressive activity and appears to be safer in humans than morphine for equi-analgsesic doses. Similarly, a series of substituted fentanyl analogues was observed to produce increased therapeutic window for respiratory depression correlating with increased G-protein versus β-arrestin 2 recruitment. PZM21, developed via in silico screening with novel receptor interactions, appears to be a G-protein biased agonist when compared to morphine. It was reported to produce no respiratory depression but this has not been reproduced by others (Hill et al Br J Pharmacol (26 Mar. 2018) epub PMID: 29582414).

To investigate whether bias could be explained by the differential interaction of bilorphin and endomorphin-2 with MOPr, or by distinct receptor conformational changes initiated by each, Molecular Dynamics simulations with bilorphin or endomorphin-2 bound to MOPr were undertaken. Both peptides were docked to the orthosteric binding site of MOPr and displayed differences in ligand-residue interactions, which may translate to their differing bias profiles. Notably, endomorphin-2 transiently interacted with residues in ECL2, including the conserved residue Leu219, proposed to be important for arrestin-bias and ligand residence time at the 5HT2A and 5HT2B receptors and other aminergic GPCRs. In contrast, bilorphin did not contact the extracellular loops. The interactions between the peptides and the MOPr binding pocket appear to translate to the divergent conformational changes observed by the PCA. Specifically, with endomorphin-2 bound the extracellular portions of the TMDs moved inwards so that the orthosteric binding pocket contracted relative to the bilorphin-bound MOPr. On the intracellular side of the receptor TM5, 6 and 7 adopted distinct positions depending on the bound peptide, mainly an inward shift of these helices in the presence of endormorphin-2. As has been previously suggested, interaction with a G protein or arrestin may be required for MOPr to achieve a fully active state, and therefore it is unsurprising that in these MD simulations of the receptor and agonist alone, the intracellular portion of the complex did not sample the fully active conformation captured in the agonist and nanobody-bound crystal structure.

Whilst it remains challenging at present to associate ligand-induced GPCR conformations with differential coupling to G proteins or arrestins, the subtle differences in ligand-residue interactions and conformations of the MOPr helices that we have modelled here may represent the initial changes induced by the oppositely biased peptides, bilorphin and endomorphin-2, which lead to their different signalling profiles and potentially adverse effect liabilities.

It remains uncertain, however, whether G-protein bias per se is the sole property contributing to improved safety of drugs such as oliceridine (TRV130) (Singla et al J Pain Res (2017) 10: 2413-2424). Using receptor knockdown, it is shown here that TRV130 has very low G-protein efficacy compared with morphine. Similar results have been reported for another opioid, PZM21, claimed to be safer than morphine (Hill et al Br J Pharmacol (26 Mar. 2018) epub PMID: 29582414) and it is difficult to evaluate G-protein efficacy of novel biased agonists in other studies because assays were insensitive to the relatively low G-protein efficacy of morphine (Schmid et al Cell (2017) 171(5): 1165-1175; DeWire et al J Pharmacol Exp Ther (2013) 344(3): 708-17). Very low G-protein efficacy may indeed be a confounding factor in the pre-clinical and clinical studies of side effect profile, given that agonists with very low G-protein efficacy such as buprenorphine are not strongly biased but are well characterised to produce less respiratory depression and overdose death than highly efficacious agonists such as morphine and methadone. Because bilorphin is strongly G-protein biased and has nearly equivalent G-protein efficacy to morphine, its analogs will facilitate the direct test of the influence of bias without being confounded by differing G-protein efficacy.

1. An isolated peptide comprising Formula I 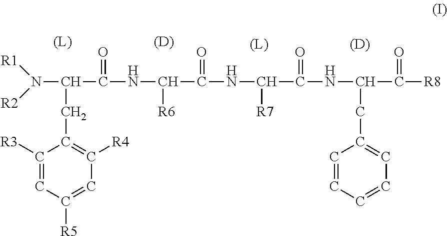
wherein, counting from the N-terminus, the first and third amino acid residues are L-amino acid residues and the second and fourth amino acid residues are D-amino acid residues; and wherein
R¹ is hydrogen, C₁-C₃ alkyl, or a bio-reversible moiety optionally comprising a sugar moiety;
R² is hydrogen, C₁-C₃ alkyl, or a bio-reversible moiety optionally comprising a sugar moiety;
wherein R¹ and R² may together form one bio-reversible moiety;
R³ and R⁴ are independently selected from hydrogen or C₁-C₃ alkyl, preferably —CH₃;
R⁵ is hydrogen, —OH, or a bio-reversible moiety optionally comprising a sugar moiety;
R⁶ is a side chain of an amino acid or C₁-C₆ alkyl;
R⁷ is a side chain of an amino acid or C₁-C₆ alkyl;
R⁸ is —OH, —NH₂, —O(C₁-C₃ alkyl), 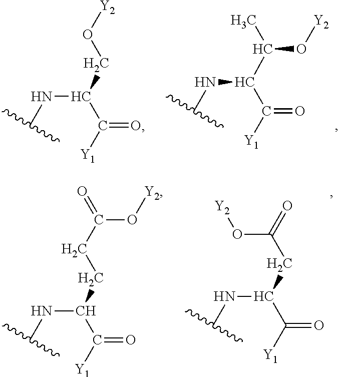
or 1 to about 30 L-amino acid residues;
Y₁ is —OH, —NH₂, or 1 to about 30 L-amino acid residues;
Y₂ is hydrogen or a sugar moiety, preferably a disaccharide moiety; and
wherein when R⁸ is 1 to about 30 L-amino acid residues (1) the L-amino acid residues are optionally residues that may be optionally glycosylated with a sugar moiety, preferably a disaccharide moiety, and (2) the C-terminus is optionally amidated.

2. The peptide according to claim 1, wherein R⁶ is C₁-C₆ alkyl and R⁷ is C₁-C₆ alkyl; and/or
wherein R⁶ and R⁷ are independently selected from the side chain of alanine, valine, norvaline, leucine, norleucine, or isoleucine; and/or
wherein R⁶ and R⁷ are each a valine side chain (—CH(CH₃)₂); and/or
wherein R⁶ and R⁷ are each a threonine side chain; and/or
wherein R³ and R⁴ are —CH₃; and R⁵ is —OH.

3.-8. (canceled)

9. An isolated peptide comprising Formula I 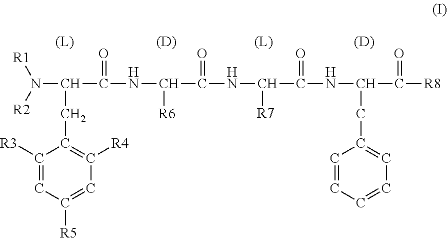
wherein, counting from the N-terminus, the first and third amino acid residues are L-amino acid residues and the second and fourth amino acid residues are D-amino acid residues; and wherein
R¹ is hydrogen, single bond, or a —C₁-C₃ alkyl;
R² is hydrogen, single bond, or a —C₁-C₃ alkyl;
R³ and R⁴ are independently selected from hydrogen or C₁-C₃ alkyl, preferably —CH₃;
R⁵ is hydrogen, —OH, or —O(C₁-C₃)alkyl;
R⁶ is a side chain of an amino acid or C₁-C₆ alkyl;
R⁷ is a side chain of an amino acid or C₁-C₆ alkyl;
R⁸ is —OH, —NH₂, —O(C₁-C₃ alkyl), 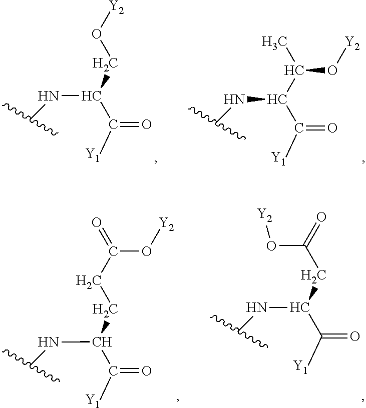
or 1 to about 30 L-amino acid residues;
Y₁ is —OH, —NH₂, or 1 to about 30 L-amino acid residues;
Y₂ is hydrogen or a sugar moiety, preferably a disaccharide moiety; and
wherein when R⁸ is 1 to about 30 L-amino acid residues (1) the L-amino acid residues are optionally residues that may be optionally glycosylated with a sugar moiety, preferably a disaccharide moiety, and (2) the C-terminus is optionally amidated;
wherein when R⁸ is a linker, the linker comprises a sugar moiety, preferably a disaccharide moiety such as lactose, and
wherein when one of R¹ or R² is a single bond, one of R¹ and R² is hydrogen and the single bond is a peptide bond to an L-amino acid residue that may optionally be N-terminally alkylated, preferably singly methylated.

10. The peptide according to claim 9, wherein one of R¹ and R² is hydrogen and one of R¹ and R² is —CH₃; and/or
wherein R⁵ is —O(C₁-C₃)alkyl, preferably —OCH₃; preferably wherein R³ and R⁴ are —CH₃; or
wherein R³ and R⁴ are —CH₃ and R⁵ is —OH; and/or
wherein R⁶ and R⁷ are each a valine side chain (—CH(CH₃)₂); or
wherein R⁶ and R⁷ are each a threonine side chain; and/or
wherein one of R¹ or R² is a single bond, one of R¹ and R² is a hydrogen, and the single bond is a peptide bond to an L-amino acid residue; optionally wherein the L-amino acid residue has at least one N-terminal methylation; and/or optionally wherein the L-amino acid residue is an L-alanine residue.

11.-21. (canceled)

22. The peptide according to claim 1, wherein R⁸ is —OH, —NH₂, —O(C₁-C₃ alkyl), 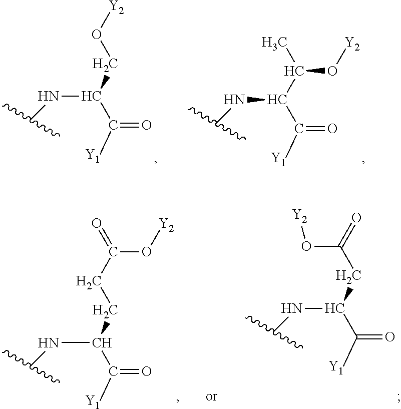
Y₁ is —OH, —NH₂, or 1 to about 30 L-amino acid residues; and
Y₂ is hydrogen or a sugar moiety, preferably a disaccharide moiety; and
wherein R⁸ is 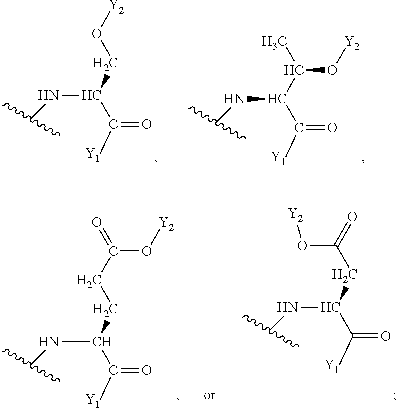
Y₁ is —OH, —NH₂, or 1 to about 30 L-amino acid residues; and
Y₂ is hydrogen or a sugar moiety, preferably a disaccharide moiety; or
wherein Y₁—NH₂; or
wherein Y₁ is 1 to about 30 L-amino acid residues; or
wherein Y₁ is 1 to about 25 L-amino acid residues, 1 to about 20 L-amino acid residues, 1 to about 15 L-amino acid residues, 1 to about 11 L-amino acid residues, or 1 to about 5 L-amino acid residues; optionally wherein Y¹ is 1 to about 11 L-amino acid residues.

23.-29. (canceled)

30. The peptide according to claim 1, wherein R⁸ is 1 to about 11 L-amino acid residues, wherein the 1 to about 11 L-amino acid residues comprise at least one glycosylated L-amino acid residue, preferably comprising at least one O-glycosylated L-serine residue.

31. (canceled)

32. The peptide according to claim 1, wherein R¹ and R² are hydrogen; R³, R⁴, and R⁵ are hydrogen; R⁶ and R⁷ are each —CH(CH₃)₂; and R⁸ is —OH, —NH₂, —O(C₁-C₃ alkyl), 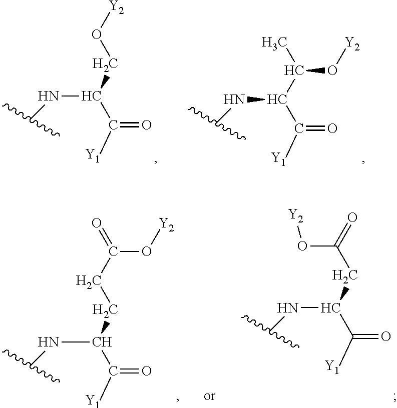
Y₁ is —OH, —NH₂, or 1 to about 30 L-amino acid residues; and
Y₂ is hydrogen or a sugar moiety, preferably a disaccharide moiety; or
wherein R¹ and R² are hydrogen; R³ and R⁴ are both hydrogen or both —CH₃; R⁵ is —OH; R⁶ and R⁷ are each —CH(CH₃)₂; and R⁸ is —OH, —NH₂, —O(C₁-C₃ alkyl), 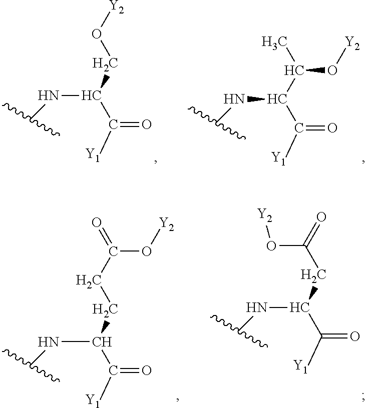
Y₁ is —OH, —NH₂, or 1 to about 30 L-amino acid residues;
Y₂ is hydrogen or a sugar moiety, preferably a disaccharide moiety; optionally wherein R⁸ is —NH₂, 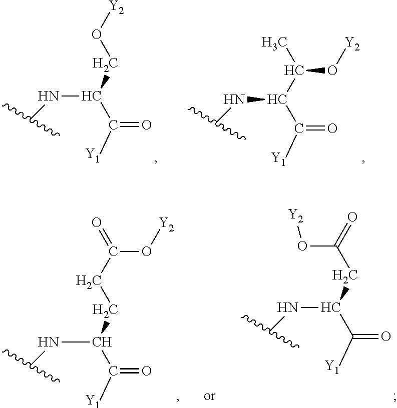
Y₁ is —OH, —NH₂, or 1 to about 30 L-amino acid residues; and
Y₂ is hydrogen or a sugar moiety, preferably a disaccharide moiety;
optionally wherein R⁸ is 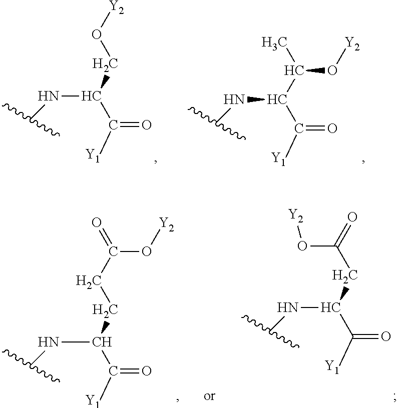
Y₁ is —OH or —NH₂; and
Y₂ is hydrogen or a sugar moiety, preferably a disaccharide moiety; or
optionally wherein Y₁ is 1 to about 25 L-amino acid residues, 1 to about 20 L-amino acid residues, 1 to about 15 L-amino acid residues, 1 to about 11 L-amino acid residues, or 1 to about 5 L-amino acid residues; preferably wherein Y¹ is 1 to about 11 L-amino acid residues.

33.-37. (canceled)

38. The peptide according to claim 1, wherein Y₂ is a sugar moiety, preferably a disaccharide moiety; optionally wherein Y₂ the disaccharide moiety is a lactose moiety or melibiose moiety; optionally wherein Y₂ the disaccharide moiety is a lactose moiety; optionally wherein the disaccharide moiety is attached through a beta linkage.

39.-41. (canceled)

42. The peptide according to claim 1, wherein R⁸ is 1 to about 25 L-amino acid residues, 1 to about 20 L-amino acid residues, 1 to about 15 L-amino acid residues, 1 to about 11 L-amino acid residues, or 1 to about 5 L-amino acid residues;
wherein said L-amino acid residues comprises at least one amino acid residue that is N-glycosylated, O-glycosylated, C-glycosylated, S-glycosylated, or Se-glycosylated;
optionally wherein said L-amino acid residues comprise at least one L-amino acid residue that is O-glycosylated;
optionally wherein said amino acid residue that is O-glycosylated is an L-serine residue.

43.-46. (canceled)

47. The peptide according to claim 1, wherein R¹ and R² together form a bio-reversible moiety;
optionally wherein said bio-reversible moiety is 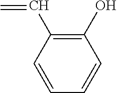
or ═N═N (azido moiety) or
the peptide according to claim 1 wherein one of R¹ or R² is hydrogen and one of R¹ or R² is —C(═O)OCH₂CH₃ or —C(═O)OCH₂OC(═O)CH₃; or
wherein R⁵ is a bio-reversible moiety
optionally wherein the bio-reversible moiety is —C(═O)CH₃.

48.-51. (canceled)

52. The peptide according to claim 1, selected from the group consisting of:
L-Phe-D-Val-L-Val-D-Phe (peptide 1a, Bilaid A);
L-Phe-D-Val-L-Val-D-Phe-NH₂ (peptide 1e);
L-Tyr-D-Val-L-Val-D-Phe (peptide 3a, Bilaid C);
L-Tyr-D-Val-L-Val-D-Phe-NH₂ (peptide 3b);
2,6-dimethyl-L-tyrosine-D-Val-L-Val-D-Phe-NH₂ (peptide 3c; Bilorphin);
2,6-dimethyl-L-tyrosine-D-Val-L-Val-D-Phe-L-Ser(β-Lac)-NH₂ (peptide 3g; Bilactorphin);
2,6-dimethyl-L-tyrosine-D-Val-L-Val-D-Phe-OCH₂CH₃ (peptide 4);
2,6-dimethyl-L-tyrosine-D-Val-L-Val-D-Phe-L-Pro-L-Asn-L-Leu-L-Ala-L-Glu-L-Lys-L-Ala-L-Leu-L-Lys-L-Ser-L-Leu-NH₂ (peptide 11);
2,6-dimethyl-L-tyrosine-D-Val-L-Val-D-Phe-NH₂ wherein the N-terminus is substituted with the bio-reversible moiety —C(═O)OCH₂OC(═O)CH₃ (peptide 10);
2,6-dimethyl-L-tyrosine-D-Val-L-Val-D-Phe-NH₂ wherein the N-terminus is substituted with the bio-reversible moiety 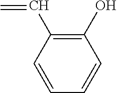
(peptide 8);
2,6-dimethyl-L-tyrosine-D-Val-L-Val-D-Phe-NH₂ wherein the hydroxy group on 2,6-dimethyl-L-tyrosine is substituted with the bio-reversible moiety —C(═O)CH₃ (peptide 5);
2,6-dimethyl-L-tyrosine-D-Val-L-Val-D-Phe-OCH₂CH₃ wherein the hydroxy group on 2,6-dimethyl-L-tyrosine is substituted with the bio-reversible moiety —C(═O)CH₃ (peptide 6);
2,6-dimethyl-L-tyrosine-D-Val-L-Val-D-Phe-NH₂ wherein the N-terminus is substituted with the bio-reversible moiety —C(═O)OCH₂CH₃ (peptide 7); and
2,6-dimethyl-L-tyrosine-D-Val-L-Val-D-Phe-NH₂ wherein the N-terminus is substituted with the bio-reversible moiety, ═N═N, to form an N-terminal azido group (peptide 9).

53. A peptide comprising:
L-AA-L-Tyr-D-Val-L-Val-D-Phe-linker-sugar moiety;
L-AA-L-Tyr-D-Thr-L-Thr-D-Phe-linker-sugar moiety;
L-AA-L-Dmt-D-Val-L-Val-D-Phe-linker-sugar moiety;
L-AA-L-Dmt-D-Thr-L-Thr-D-Phe-linker-sugar moiety;
wherein L-AA is any L-amino acid residue optionally with at least one N-terminal —CH₃;
wherein the hydroxyl group of L-Tyr or L-Dmt is optionally alkylated; and
wherein the linker is preferably L-Ser or L-Thr.

54. A peptide comprising Formula II 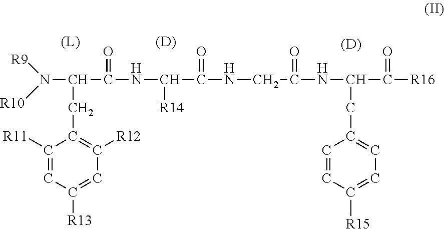
wherein, counting from the N-terminus, the first amino acid residue is an L-amino acid residue and the second and fourth amino acid residues are D-amino acid residues; wherein
R⁹ is hydrogen or a bio-reversible moiety optionally comprising a sugar moiety;
R¹⁰ is hydrogen or a bio-reversible moiety optionally comprising a sugar moiety;
wherein when one of R⁹ or R¹⁰ is a hydrogen and one of R⁹ or R¹⁰ is a bio-reversible moiety, the bio-reversible moiety is preferably —C(═O)OCH₂CH₃ or —C(═O)OCH₂OC(═O)CH₃;
wherein R⁹ and R¹⁰ may together form one bio-reversible moiety, wherein preferably the bio-reversible moiety is 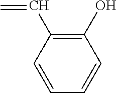
or ═N═N (azido moiety);
R¹¹ and R¹² are independently selected from hydrogen or C₁-C₃ alkyl, preferably —CH₃;
R¹³ is hydrogen, —OH, or a bio-reversible moiety optionally comprising a sugar moiety;
R¹⁴ is a side chain of an amino acid or C₁-C₆ alkyl, preferably C₁-C₄ alkyl, more preferably —CH(CH₃)₂;
R¹⁵ is hydrogen, —OH, or a bio-reversible moiety; and
R¹⁶ is —OH, —O(C₁-C₃ alkyl), —NH₂, 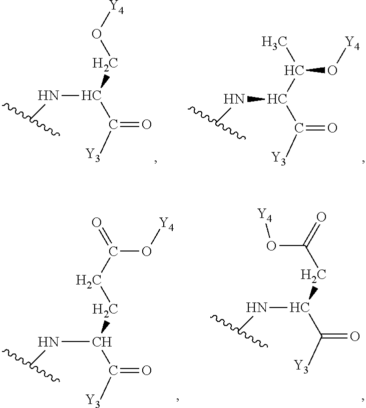
or 1 to about 30 L-amino acid residues;
Y₃ is —OH, —NH₂, or 1 to about 30 L-amino acid residues;
Y₄ is hydrogen or a sugar moiety, preferably a disaccharide moiety; and
wherein when R¹⁶ is 1 to about 30 L-amino acid residues (1) the L-amino acid residues are optionally residues that may be optionally glycosylated with a sugar moiety, preferably a disaccharide moiety, and (2) the C-terminus is optionally amidated.

55. (canceled)

56. (canceled)

57. The peptide according to claim 54, wherein R¹⁴ is a valine side chain (—CH(CH₃)₂); or
wherein R¹⁴ is a threonine side chain;
wherein optionally R¹¹ and R¹² are —CH₃; and R¹³ is —OH;
wherein optionally R⁹, R¹⁰, R¹¹, R¹², and R¹³ are each hydrogen;
wherein optionally R⁹, R¹⁰, R¹¹, R¹², and R¹³ are hydrogen; R¹⁴ is C₁-C₄ alkyl; R¹⁵ is —OH.

58.-62. (canceled)

63. A peptide comprising Formula II 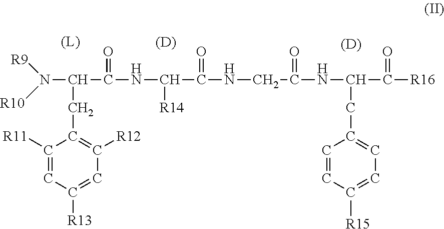
wherein, counting from the N-terminus, the first amino acid residue is an L-amino acid residue and the second and fourth amino acid residues are D-amino acid residues; wherein
R⁹ is hydrogen, a single bond, or —C₁-C₃ alkyl, preferably —CH₃;
R¹⁰ is hydrogen, a single bond, or —C₁-C₃ alkyl, preferably —CH₃;
R¹¹ and R¹² are independently selected from hydrogen or C₁-C₃ alkyl, preferably —CH₃;
R¹³ is hydrogen, —OH, or —O(C₁-C₃)alkyl;
R¹⁴ is a side chain of an amino acid or C₁-C₆ alkyl, preferably C₁-C₄ alkyl, more preferably —CH(CH₃)₂;
R¹⁵ is hydrogen, —OH, or a bio-reversible moiety; and
R¹⁶ is —OH, —O(C₁-C₃ alkyl), —NH₂, 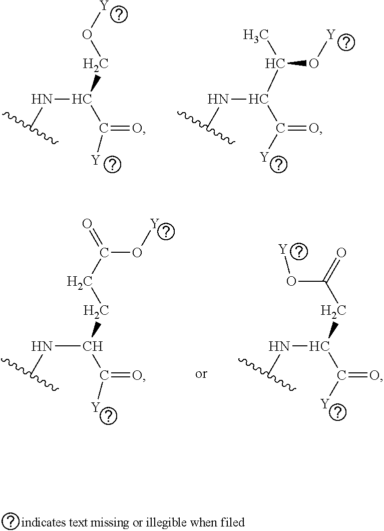
1 to about 30 L-amino acid residues, or a linker;
Y₃ is —OH, —NH₂, or 1 to about 30 L-amino acid residues;
Y₄ is hydrogen or a sugar moiety, preferably a disaccharide moiety;
wherein when R¹⁶ is 1 to about 30 L-amino acid residues (1) the L-amino acid residues are optionally residues that may be optionally glycosylated with a sugar moiety, preferably a disaccharide moiety, and (2) the C-terminus is optionally amidated;
wherein when R¹⁶ is a linker, the linker comprises a sugar moiety, preferably a disaccharide moiety such as lactose, and
wherein when one of R⁹ or R¹⁰ is a single bond, one of R⁹ or R¹⁰ is hydrogen and the single bond is a peptide bond to an L-amino acid residue optionally N-terminally alkylated, preferably singly methylated.

64. The peptide according to claim 63, wherein one of R⁹ and R¹⁰ is hydrogen and one of R⁹ and R¹⁰ is —CH₃; or
wherein R¹³ is —O(C₁-C₃)alkyl, preferably —OCH₃,
optionally wherein R¹¹ and R¹² are —CH₃; or
wherein R¹¹ and R¹² are —CH₃, and R¹³ is —OH; or
wherein one of R⁹ or R¹⁰ is a single bond, one of R⁹ or R¹⁰ is hydrogen, and the single bond is a peptide bond to an L-amino acid residue; optionally wherein the L-amino acid residue has at least one N-terminal methylation; and/or optionally wherein the L-amino acid residue is an L-alanine residue; or
wherein R¹⁴ is a valine side chain (—CH(CH₃)₂); or
wherein R¹⁴ is threonine side chain.

65.-73. (canceled)

74. The peptide according to claim 54, wherein R¹⁶ is —NH₂.

75. The peptide according to claim 54, wherein
R¹⁶ is 1 to about 30 L-amino acid residues;
Y₃ is —OH, —NH₂, or 1 to about 30 L-amino acid residues;
Y₄ is hydrogen or a sugar moiety, preferably a disaccharide moiety; and
wherein when R¹⁶ is 1 to about 30 L-amino acid residues (1) the L-amino acid residues are optionally residues that may be optionally glycosylated with a sugar moiety, preferably a disaccharide moiety, and (2) the C-terminus is optionally amidated;
wherein said L-amino acid residues comprises at least one amino acid residue that is N-glycosylated, O-glycosylated, C-glycosylated, S-glycosylated, or Se-glycosylated;
optionally wherein said L-amino acid residues comprise at least one amino acid residue that is O-glycosylated;
optionally wherein said amino acid residue that is O-glycosylated is an L-serine residue.

76.-83. (canceled)

84. The peptide according to claim 54, wherein
R¹⁶ is 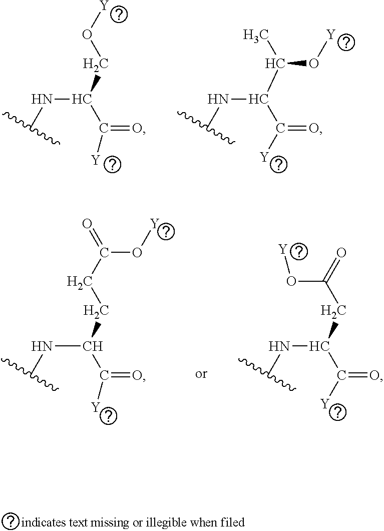
Y₃ is —OH, —NH₂, or 1 to about 30 L-amino acid residues; and
Y₄ is hydrogen or a sugar moiety, preferably a disaccharide moiety; or
wherein R⁹, R¹⁰, R¹¹, R¹², and R¹³ are hydrogen; R¹⁴ is —CH(CH₃)₂; R¹⁵ is —OH; and
R¹⁶ is 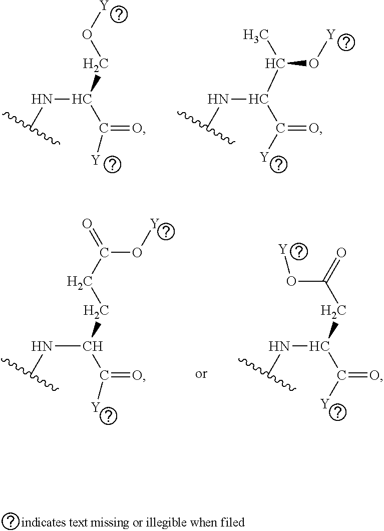
Y₃ is —OH or —NH₂; and Y₄ is hydrogen or a sugar moiety, preferably a disaccharide moiety; or
wherein the peptide is L-Phe-D-Val-Gly-D-Tyr-NH₂.

85. The peptide according to claim 54, wherein
R¹⁶ is 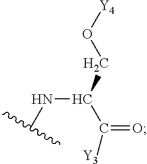
Y₃ is —OH or —NH₂; and Y₄ is hydrogen or a sugar moiety, preferably a disaccharide moiety; or
the peptide according to claim 54, wherein said sugar moiety is a disaccharide moiety, preferably wherein the disaccharide moiety is attached through a beta linkage;
optionally wherein said disaccharide moiety is a lactose moiety or a melibiose moiety, preferably wherein the disaccharide moiety is attached through a beta linkage;
optionally wherein said disaccharide moiety is a lactose moiety, preferably wherein the lactose moiety is attached through a beta linkage; or
the peptide according to claim 54, wherein R⁹ and R¹⁰ together form a bio-reversible moiety;
optionally wherein said bio-reversible moiety is 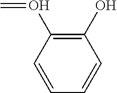
or ═N═N (azido moiety); or
wherein one of R⁹ or R¹⁰ is hydrogen and one of R⁹ or R¹⁰ is —C(═O)OCH₂CH₃ or —C(═O)OCH₂OC(═O)CH₃; or
wherein R¹³ is a bio-reversible moiety; optionally wherein the bio-reversible moiety is —C(═O)CH₃.

86.-95. (canceled)

96. An isolated peptide comprising Formula III
X¹—X²—X³—X⁴ (III)
wherein:
X¹ is the N-terminal amino acid residue comprising an N-terminal moiety —NR¹⁷R¹⁸;
X⁴ comprises a C-terminal moiety selected from —C(═O)OH, —C(═O)O(C₁-C₃ alkyl) —C(═O)NH₂, 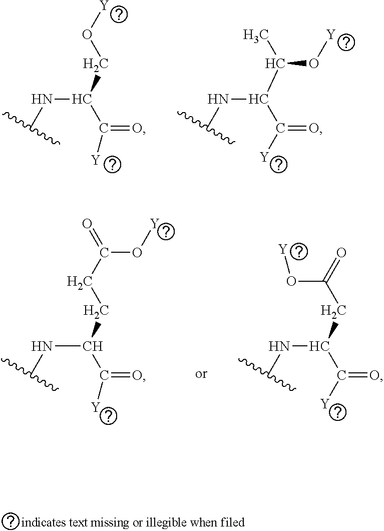
wherein Y₅ is —OH or —NH₂, and Y₆ is hydrogen or a sugar moiety, preferably a disaccharide moiety;
X¹ is an L-amino acid residue selected from L-tyrosine, 2,6-dimethyl-L-tyrosine, or L-phenylalanine, wherein when X¹ is L-tyrosine or 2,6-dimethyl-L-tyrosine, the residue is optionally O-substituted at the 4-position with a bio-reversible moiety optionally comprising a sugar moiety;
X₂ is a D-amino acid residue, preferably D-alanine, D-valine, D-leucine, or D-isoleucine, more preferably, D-valine;
X₃ is glycine or an L-amino acid residue, wherein when X₃ is an L-amino acid residue, X₃ is preferably L-alanine, L-valine, L-leucine, or L-isoleucine, more preferably L-valine;
X₄ is a D-amino acid residue selected from D-tyrosine or D-phenylalanine, wherein when X₄ is D-tyrosine, the residue is optionally O-substituted with a bio-reversible moiety;
R¹⁷ and R¹⁸ are independently selected from hydrogen or a bio-reversible moiety optionally comprising a sugar moiety, or R¹⁷ and R¹⁸ together form a bio-reversible moiety optionally comprising a sugar moiety; and
wherein the peptide is a MOPr agonist.

97. The peptide according to claim 96, wherein the C-terminal moiety is —C(═O)OH, 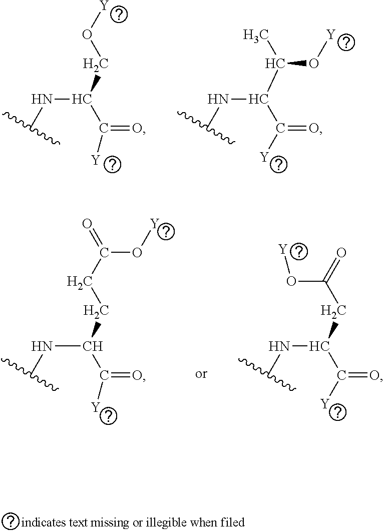
and Y₅ is —OH and Y₆ is hydrogen or a sugar moiety; the peptide further comprises about 5, 8, 11, 12, 20 or 26 additional L-amino acid residues on the C-terminus; and/or
wherein R¹⁷ and R¹⁸ are each hydrogen, X² is a D-valine residue, X³ is glycine or an L-valine residue, X⁴ comprises a C-terminal moiety selected from —C(═O)OH, —C(═O)NH₂, 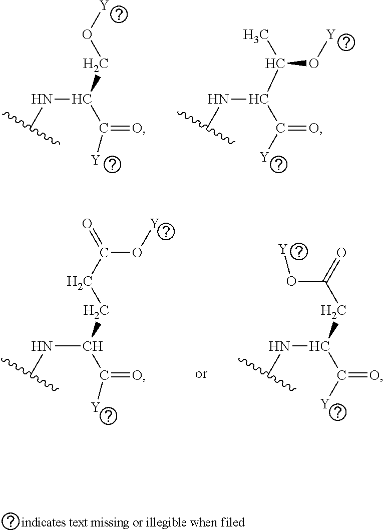
wherein Y₅ is —OH or —NH₂, and Y₆ is hydrogen or a sugar moiety, preferably a disaccharide moiety.

98. (canceled)

99. An isolated peptide comprising Formula III
X¹—X²—X³—X⁴ (III)
wherein:
X¹ is the N-terminal amino acid residue comprising an N-terminal moiety —NR¹⁷R¹⁸;
X⁴ comprises a C-terminal moiety selected from —C(═O)OH, —C(═O)O(C₁-C₃ alkyl)-C(═O)NH₂, 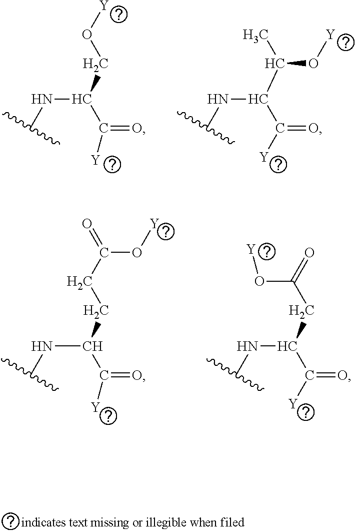
or a linker, wherein Y₅ is —OH or —NH₂, and Y₆ is hydrogen or a sugar moiety, preferably a disaccharide moiety;
X¹ is an L-amino acid residue selected from L-tyrosine, 2,6-dimethyl-L-tyrosine, or L-phenylalanine, wherein when X¹ is L-tyrosine or 2,6-dimethyl-L-tyrosine, the residue is optionally O-substituted at the 4-position with C₁-C₃ alkyl;
X² is a D-amino acid residue, preferably D-threonine, D-alanine, D-valine, D-leucine, or D-isoleucine, more preferably, D-threonine or D-valine;
X³ is glycine or an L-amino acid residue, wherein when X³ is an L-amino acid residue, X³ is preferably L-threonine, L-alanine, L-valine, L-leucine, or L-isoleucine, more preferably L-threonine or L-valine;
X⁴ is a D-amino acid residue selected from D-tyrosine or D-phenylalanine, wherein when X⁴ is D-tyrosine, the residue is optionally O-substituted with a bio-reversible moiety;
R¹⁷ and R¹⁸ are independently selected from hydrogen, a single bond, or a —C₁-C₃ alkyl, preferably —CH₃; and
wherein when X⁴ comprises a linker, the linker comprises a sugar moiety, preferably a disaccharide moiety such as lactose,
wherein when one of R¹⁷ or R¹⁸ is a single bond, one of R¹⁷ or R¹⁸ is hydrogen and the single bond is a peptide bond to an L-amino acid residue that may optionally be N-terminally alkylated, preferably singly methylated; and
wherein the peptide is a MOPr agonist.

100. The peptide according to claim 99, wherein the C-terminal moiety is —C(═O)OH, 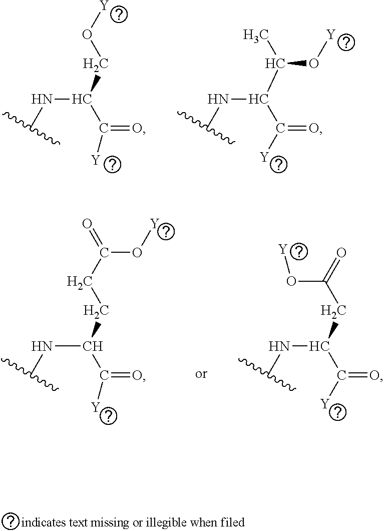
and Y₅ is —OH and Y₆ is hydrogen or a sugar moiety; the peptide further comprises about 5, 8, 11, 12, 20 or 26 additional L-amino acid residues on the C-terminus; and/or
wherein X² is a D-valine residue, X³ is glycine or an L-valine residue, X⁴ comprises a C-terminal moiety selected from —C(═O)OH, —C(═O)NH₂, 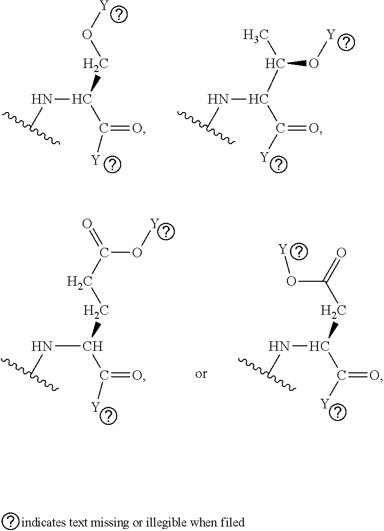
wherein Y₅ is —OH or —NH₂, and Y₆ is hydrogen or a sugar moiety, preferably a disaccharide moiety; and/or
wherein X² is a D-threonine residue, X³ is glycine or an L-threonine residue, X⁴ comprises a C-terminal moiety selected from —C(═O)OH, —C(═O)NH₂, 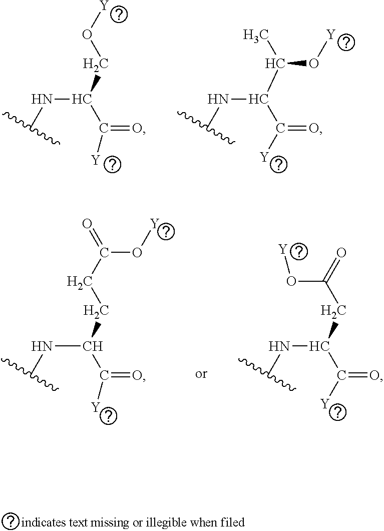
wherein Y₅ is —OH or —NH₂, and Y₆ is hydrogen or a sugar moiety, preferably a disaccharide moiety; and/or
wherein one of R¹⁷ and R¹⁸ is hydrogen and one of R¹⁷ and R¹⁸ is —CH₃; or
wherein one of R¹⁷ or R¹⁸ is a single bond, one of R¹⁷ or R¹⁸ is a hydrogen, and the single bond is a peptide bond to an L-amino acid residue; optionally wherein the L-amino acid residue has at least one N-terminal methylation; and/or
wherein the L-amino acid residue is an L-alanine residue; or
wherein X⁴ comprises a linker; optionally wherein the linker comprises an amino acid based linker, peptide based linker, an amino acid comprising linker, and/or maleimide based linker, and/or a combination thereof; and/or
wherein X¹ is L-tyrosine or 2,6-dimethyl-L-tyrosine and wherein the L-tyrosine or 2,6-dimethyl-L-tyrosine is O-substituted at the 4-position with C₁-C₃ alkyl; optionally wherein X¹ is 2,6-dimethyl-L-tyrosine and wherein 2,6-dimethyl-L-tyrosine is O-substituted at the 4-position with C₁-C₃ alkyl.

101.-110. (canceled)

111. The peptide according to claim 96, wherein X⁴ comprises a C-terminal moiety selected from, 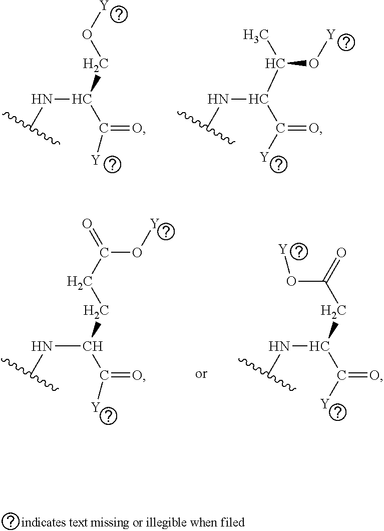
wherein Y₅ is —OH or —NH₂, and Y₆ is a disaccharide moiety, preferably wherein the disaccharide moiety is attached through a beta linkage; optionally wherein the disaccharide moiety is a lactose moiety, preferably wherein the lactose moiety is attached through a beta linkage; or
wherein said additional L-amino acids comprise at least one amino acid residue that is N-glycosylated, O-glycosylated, C-glycosylated, S-glycosylated, or Se-glycosylated; or
wherein R¹⁷ and R¹⁸ together form a bio-reversible moiety; optionally wherein said bio-reversible moiety is 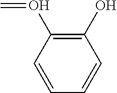
or ═N═N (azido moiety); or
wherein one of R¹⁷ or R¹⁸ is hydrogen and one of R¹⁷ or R¹⁸ is —C(═O)OCH₂CH₃ or —C(═O)OCH₂OC(═O)CH₃.

112.-116. (canceled)

117. The peptide according to claim 96 selected from the group consisting of
L-Phe-D-Val-L-Val-D-Phe (peptide 1a, Bilaid A);
L-Phe-D-Val-L-Val-D-Phe-NH₂ (peptide 1e);
L-Tyr-D-Val-L-Val-D-Phe (peptide 3a, Bilaid C);
L-Tyr-D-Val-L-Val-D-Phe-NH₂ (peptide 3b);
2,6-dimethyl-L-tyrosine-D-Val-L-Val-D-Phe-NH₂ (peptide 3c; Bilorphin);
2,6-dimethyl-L-tyrosine-D-Val-L-Val-D-Phe-L-Ser(β-Lac)-NH₂ (peptide 3g; Bilactorphin);
L-Phe-D-Val-Gly-D-Tyr-NH₂ (peptide 2d);
2,6-dimethyl-L-tyrosine-D-Val-L-Val-D-Phe-OCH₂CH₃ (peptide 4);
2,6-dimethyl-L-tyrosine-D-Val-L-Val-D-Phe-L-Pro-L-Asn-L-Leu-L-Ala-L-Glu-L-Lys-L-Ala-L-Leu-L-Lys-L-Ser-L-Leu-NH₂ (peptide 11);
2,6-dimethyl-L-tyrosine-D-Val-L-Val-D-Phe-NH₂ wherein the N-terminus is substituted with the bio-reversible moiety —C(═O)OCH₂OC(═O)CH₃ (peptide 10);
2,6-dimethyl-L-tyrosine-D-Val-L-Val-D-Phe-NH₂ wherein the N-terminus is substituted with the bio-reversible moiety 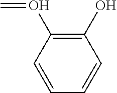
(peptide 8);
2,6-dimethyl-L-tyrosine-D-Val-L-Val-D-Phe-NH₂ wherein the hydroxy group on 2,6-dimethyl-L-tyrosine is substituted with the bio-reversible moiety —C(═O)CH₃ (peptide 5);
2,6-dimethyl-L-tyrosine-D-Val-L-Val-D-Phe-OCH₂CH₃ wherein the hydroxy group on 2,6-dimethyl-L-tyrosine is substituted with the bio-reversible moiety —C(═O)CH₃ (peptide 6);
2,6-dimethyl-L-tyrosine-D-Val-L-Val-D-Phe-NH₂ wherein the N-terminus is substituted with the bio-reversible moiety —C(═O)OCH₂CH₃ (peptide 7); and
2,6-dimethyl-L-tyrosine-D-Val-L-Val-D-Phe-NH₂ wherein the N-terminus is substituted with the bio-reversible moiety, ═N═N, to form an N-terminal azido group (peptide 9).

118.-123. (canceled)

124. A pharmaceutical composition comprising a peptide according to claim 1 and at least one pharmaceutical excipient; wherein optionally
(i) said composition is formulated for oral administration; or
(ii) said peptide is not glycosylated and said composition is formulated for nasal administration or intrathecal administration.

125. (canceled)

126. The pharmaceutical composition according to claim 124, wherein said peptide is glycosylated and said composition is formulated for oral administration, administration by injection, or intrathecal administration.

127.-134. (canceled)

135. A method of:
(i) treating pain, preferably wherein the pain is post-operative pain, pain associated with nerve damage, pain associated with bone fracture, pain associated with a burn, or pain associated with a wound;
(ii) delivering analgesia; or
(iii) treating pain or delivering analgesia with reduced adverse side effect(s), preferably reduced in comparison to morphine and preferably wherein said adverse side effect(s) is gastrointestinal (GI) inhibition and/or respiratory depression,
in a subject in need thereof, comprising administering to the subject the peptide according to claim 1.

136.-138. (canceled)


SEQUENCE LISTING (31 sequences)

SEQ ID NO: 1 - protein, 4 aa, Penicillium
  MISC_FEATURE (2)..(2): D-amino acid (D-Val)
  MISC_FEATURE (4)..(4): D-amino acid (D-Phe)
FVVF

SEQ ID NO: 2 - protein, 4 aa, Penicillium
  MISC_FEATURE (2)..(2): D-amino acid (D-Val)
  MISC_FEATURE (4)..(4): D-amino acid (D-Tyr)
FVVY

SEQ ID NO: 3 - protein, 4 aa, Penicillium
  MISC_FEATURE (2)..(2): D-amino acid (D-Val)
  MISC_FEATURE (4)..(4): D-amino acid (D-Phe)
YVVF

SEQ ID NO: 4 - protein, 4 aa, Artificial Sequence
  Table 1 peptide 1b
  MISC_FEATURE (1)..(1): D-amino acid (D-Phe)
  MISC_FEATURE (3)..(3): D-amino acid (D-Val)
FVVF

SEQ ID NO: 5 - protein, 4 aa, Artificial Sequence
  Table 1 peptide 1c
  MISC_FEATURE (3)..(3): D-amino acid (D-Val)
  MISC_FEATURE (4)..(4): D-amino acid (D-Phe)
FVVF

SEQ ID NO: 6 - protein, 4 aa, Artificial Sequence
  Table 1 peptide 1d
  MISC_FEATURE (1)..(1): D-amino acid (D-Phe)
  MISC_FEATURE (2)..(2): D-amino acid (D-Val)
FVVF

SEQ ID NO: 7 - protein, 4 aa, Artificial Sequence
  Table 1 peptide 1e
  MISC_FEATURE (2)..(2): D-amino acid (D-Val)
  MISC_FEATURE (4)..(4): D-amino acid (D-Phe)
  MISC_FEATURE (4)..(4): C-terminal carboxamide
FVVF

SEQ ID NO: 8 - protein, 4 aa, Artificial Sequence
  Table 1 peptide 1f
  MISC_FEATURE (1)..(1): D-amino acid (D-Phe)
  MISC_FEATURE (3)..(3): D-amino acid (D-Val)
  MISC_FEATURE (4)..(4): C-terminal carboxamide
FVVF

SEQ ID NO: 9 - protein, 4 aa, Artificial Sequence
  Table 1 peptide 1g
  MISC_FEATURE (3)..(3): D-amino acid (D-Val)
  MISC_FEATURE (4)..(4): D-amino acid (D-Phe)
  MISC_FEATURE (4)..(4): C-terminal carboxamide
FVVF

SEQ ID NO: 10 - protein, 4 aa, Artificial Sequence
  Table 1 peptide 1h
  MISC_FEATURE (4)..(4): C-terminal carboxamide
FVVF

SEQ ID NO: 11 - protein, 4 aa, Artificial Sequence
  Table 1 peptide 2b
  MISC_FEATURE (2)..(2): D-amino acid (D-Val)
  MISC_FEATURE (4)..(4): D-amino acid (D-Tyr)
  MISC_FEATURE (4)..(4): C-terminal carboxamide
FVVY

SEQ ID NO: 12 - protein, 4 aa, Artificial Sequence
  Table 1 peptide 2c
  MISC_FEATURE (4)..(4): C-terminal carboxamide
FVVY

SEQ ID NO: 13 - protein, 4 aa, Artificial Sequence
  Table 1 peptide 2d
  MISC_FEATURE (2)..(2): D-amino acid (D-Val)
  MISC_FEATURE (4)..(4): D-amino acid (D-Tyr)
  MISC_FEATURE (4)..(4): C-terminal carboxamide
FVGY

SEQ ID NO: 14 - protein, 4 aa, Artificial Sequence
  Table 1 peptide 2e
  MISC_FEATURE (4)..(4): D-amino acid (D-Tyr)
  MISC_FEATURE (4)..(4): C-terminal carboxamide
FGVY

SEQ ID NO: 15 - protein, 4 aa, Artificial Sequence
  Table 1 peptide 2f
  MISC_FEATURE (4)..(4): D-amino acid (D-Tyr)
  MISC_FEATURE (4)..(4): C-terminal carboxamide
FGGY

SEQ ID NO: 16 - protein, 4 aa, Artificial Sequence
  Table 1 peptide 3b
  MISC_FEATURE (2)..(2): D-amino acid (D-Val)
  MISC_FEATURE (4)..(4): D-amino acid (D-Phe)
  MISC_FEATURE (4)..(4): C-terminal carboxamide
YVVF

SEQ ID NO: 17 - protein, 4 aa, Artificial Sequence
  Table 1 peptide 3c
  MISC_FEATURE (1)..(1): 2,6-Dimethyl-L-tyrosine
  MISC_FEATURE (2)..(2): D-amino acid (D-Val)
  MISC_FEATURE (4)..(4): D-amino acid (D-Phe)
  MISC_FEATURE (4)..(4): C-terminal carboxamide
XVVF

SEQ ID NO: 18 - protein, 4 aa, Artificial Sequence
  Table 1 peptide 3d
  MISC_FEATURE (1)..(1): N-Terminal acylated
  MISC_FEATURE (2)..(2): D-amino acid (D-Val)
  MISC_FEATURE (4)..(4): D-amino acid (D-Phe)
  MISC_FEATURE (4)..(4): C-terminal carboxamide
YVVF

SEQ ID NO: 19 - protein, 4 aa, Artificial Sequence
  Table 1 peptide 3e
YVVF

SEQ ID NO: 20 - protein, 4 aa, Artificial Sequence
  Table 1 peptide 3f
  MISC_FEATURE (4)..(4): C-terminal carboxamide
YVVF

SEQ ID NO: 21 - protein, 5 aa, Artificial Sequence
  Table 1 peptide 3g
  MISC_FEATURE (1)..(1): 2,6-Dimethyl-L-tyrosine
  MISC_FEATURE (2)..(2): D-amino acid (D-Val)
  MISC_FEATURE (4)..(4): D-amino acid (D-Phe)
  MISC_FEATURE (5)..(5): Serine hydroxyl glycosylated (beta linkage to lactose moiety)
  MISC_FEATURE (5)..(5): C-terminal carboxamide
XVVFS

SEQ ID NO: 22 - protein, 5 aa, Artificial Sequence
  Table 1 peptide 3h
  MISC_FEATURE (1)..(1): 2,6-Dimethyl-L-tyrosine
  MISC_FEATURE (2)..(2): D-amino acid (D-Val)
  MISC_FEATURE (4)..(4): D-amino acid (D-Phe)
  MISC_FEATURE (5)..(5): Serine hydroxyl glycosylated (beta linkage to D-glucose moiety)
  MISC_FEATURE (5)..(5): C-terminal carboxamide
XVVFS

SEQ ID NO: 23 - protein, 4 aa, Artificial Sequence
  Peptide 4
  MISC_FEATURE (1)..(1): 2,6-Dimethyl-L-tyrosine
  MISC_FEATURE (2)..(2): D-amino acid (D-Val)
  MISC_FEATURE (4)..(4): D-amino acid (D-Phe)
  MISC_FEATURE (4)..(4): C-terminal ethoxy
XVVF

SEQ ID NO: 24 - protein, 4 aa, Artificial Sequence
  Peptide 5
  MISC_FEATURE (1)..(1): 2,6-Dimethyl-L-tyrosine with 4-hydroxy group acylated
  MISC_FEATURE (2)..(2): D-amino acid (D-Val)
  MISC_FEATURE (4)..(4): D-amino acid (D-Phe)
  MISC_FEATURE (4)..(4): C-terminal carboxamide
XVVF

SEQ ID NO: 25 - protein, 4 aa, Artificial Sequence
  Peptide 6
  MISC_FEATURE (1)..(1): 2,6-Dimethyl-L-tyrosine with 4-hydroxy group acylated
  MISC_FEATURE (2)..(2): D-amino acid (D-Val)
  MISC_FEATURE (4)..(4): D-amino acid (D-Phe)
  MISC_FEATURE (4)..(4): C-Terminal ethoxy
XVVF

SEQ ID NO: 26 - protein, 4 aa, Artificial Sequence
  Peptide 7
  MISC_FEATURE (1)..(1): 2,6-Dimethyl-L-tyrosine
  MISC_FEATURE (1)..(1): N-Terminal ethyl carbamate group
  MISC_FEATURE (2)..(2): D-amino acid (D-Val)
  MISC_FEATURE (4)..(4): D-amino acid (D-Phe)
  MISC_FEATURE (4)..(4): C-terminal carboxamide
XVVF

SEQ ID NO: 27 - protein, 4 aa, Artificial Sequence
  Peptide 8
  MISC_FEATURE (1)..(1): 2,6-Dimethyl-L-tyrosine
  MISC_FEATURE (1)..(1): N-terminal ortho-hydroxy-benzylideneimine group
  MISC_FEATURE (2)..(2): D-amino acid (D-Val)
  MISC_FEATURE (4)..(4): D-amino acid (D-Phe)
  MISC_FEATURE (4)..(4): C-terminal carboxamide
XVVF

SEQ ID NO: 28 - protein, 4 aa, Artificial Sequence
  Peptide 9
  MISC_FEATURE (1)..(1): N-Terminal azido group
  MISC_FEATURE (1)..(1): 2,6-Dimethyl-L-tyrosine
  MISC_FEATURE (2)..(2): D-amino acid (D-Val)
  MISC_FEATURE (4)..(4): D-amino acid (D-Phe)
  MISC_FEATURE (4)..(4): C-terminal carboxamide
XVVF

SEQ ID NO: 29 - protein, 4 aa, Artificial Sequence
  Peptide 10
  MISC_FEATURE (1)..(1): N-terminal acetoxymethoxycarbonyl (-CO2CH2OC(O)CH3) group
  MISC_FEATURE (1)..(1): 2,6-Dimethyl-L-tyrosine
  MISC_FEATURE (2)..(2): D-amino acid (D-Val)
  MISC_FEATURE (4)..(4): D-amino acid (D-Phe)
  MISC_FEATURE (4)..(4): C-terminal carboxamide
XVVF

SEQ ID NO: 30 - protein, 15 aa, Artificial Sequence
  Peptide 11
  MISC_FEATURE (1)..(1): 2,6-Dimethyl-L-tyrosine
  MISC_FEATURE (2)..(2): D-amino acid (D-Val)
  MISC_FEATURE (4)..(4): D-amino acid (D-Phe)
  MISC_FEATURE (15)..(15): C-terminal carboxamide
XVVFPNLAEKALKSL

SEQ ID NO: 31 - protein, 8 aa, Artificial Sequence
  MOPr selective antagonist
  MISC_FEATURE (1)..(1): D-amino acid (D-Phe)
  MISC_FEATURE (4)..(4): D-amino acid (D-Trp)
  MISC_FEATURE (7)..(7): L-Penicillamine
  MISC_FEATURE (8)..(8): C-terminal carboxamide
FCYWRTXT